SEQUENCE LISTING (124 sequences)

SEQ ID NO: 1 - protein, 335 aa, Aeromonas hydrophila
MKKWFVCLLGLVALTVQAADSRPAFSRIVMFGDSLSDTGKMYSKMRGYLPSSPPYYEGRFSNGPVWLEQLTKQFPGLTIANEAEGGATAVAYNKISWNPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNQLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIANQYEFLAH

SEQ ID NO: 2 - protein, 361 aa, Artificial Sequence
  pfam00657 consensus sequence
IVAFGDSLTDGEAYYGDSDGGGWGAGLADRLTALLRLRARPRGVDVFNRGISGRTSDGRLIVDALVALLFLAQSLGLPNLPPYLSGDFLRGANFASAGATILPTSGPFLIQVQFKDFKSQVLELRQALGLLQELLRLLPVLDAKSPDLVTIMIGTNDLITSAFFGPKSTESDRNVSVPEFKDNLRQLIKRLRSNNGARIIVLITLVILNLGPLGCLPLKLALALASSKNVDASGCLERLNEAVADFNEALRELAISKLEDQLRKDGLPDVKGADVPYVDLYSIFQDLDGIQNPSAYVYGFETTKACCGYGGRYNYNRVCGNAGLCNVTAKACNPSSYLLSFLFWDGFHPSEKGYKAVAEAL

SEQ ID NO: 3 - protein, 335 aa, Aeromonas hydrophila
MKKWFVCLLGLVALTVQAADSRPAFSRIVMFGDSLSDTGKMYSKMRGYLPSSPPYYEGRFSNGPVWLEQLTNEFPGLTIANEAEGGPTAVAYNKISWNPKYQVINNLDYEVTQFLQKDSFKPDDLVILWVGANDYLAYGWNTEQDAKRVRDAISDAANRMVLNGAKEILLFNLPDLGQNPSARSQKVVEAASHVSAYHNQLLLNLARQLAPTGMVKLFEIDKQFAEMLRDPQNFGLSDQRNACYGGSYVWKPFASRSASTDSQLSAFNPQERLAIAGNPLLAQAVASPMAARSASTLNCEGKMFWDQVHPTTVVHAALSEPAATFIESQYEFLAH

SEQ ID NO: 4 - protein, 336 aa, Aeromonas salmonicida
MKKWFVCLLGLIALTVQAADTRPAFSRIVMFGDSLSDTGKMYSKMRGYLPSSPPYYEGRFSNGPVWLEQLTKQFPGLTIANEAEGGATAVAYNKISWNPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIETQYEFLAHG

SEQ ID NO: 5 - protein, 295 aa, Streptomyces coelicolor
MPKPALRRVMTATVAAVGTLALGLTDATAHAAPAQATPTLDYVALGDSYSAGSGVLPVDPANLLCLRSTANYPHVIADTTGARLTDVTCGAAQTADFTRAQYPGVAPQLDALGTGTDLVTLTIGGNDNSTFINAITACGTAGVLSGGKGSPCKDRHGTSFDDEIEANTYPALKEALLGVRARAPHARVAALGYPWITPATADPSCFLKLPLAAGDVPYLRAIQAHLNDAVRRAAEETGATYVDFSGVSDGHDACEAPGTRWIEPLLFGHSLVPVHPNALGERRMAEHTMDVLGLD

SEQ ID NO: 6 - protein, 295 aa, Streptomyces coelicolor; identical to SEQ ID NO: 5

SEQ ID NO: 7 - protein, 238 aa, Saccharomyces cerevisiae
MDYEKFLLFGDSITEFAFNTRPIEDGKDQYALGAALVNEYTRKMDILQRGFKGYTSRWALKILPEILKHESNIVMATIFLGANDACSAGPQSVPLPEFIDNIRQMVSLMKSYHIRPIIIGPGLVDREKWEKEKSEEIALGYFRTNENFAIYSDALAKLANEEKVPFVALNKAFQQEGGDAWQQLLTDGLHFSGKGYKIFHDELLKVIETFYPQYHPKNMQYKLKDWRDVLDDGSNIMS

SEQ ID NO: 8 - protein, 347 aa, Ralstonia sp.
MNLRQWMGAATAALALGLAACGGGGTDQSGNPNVAKVQRMVVFGDSLSDIGTYTPVAQAVGGGKFTTNPGPIWAETVAAQLGVTLTPAVMGYATSVQNCPKAGCFDYAQGGSRVTDPNGIGHNGGAGALTYPVQQQLANFYAASNNTFNGNNDVVFVLAGSNDIFFWTTAAATSGSGVTPAIATAQVQQAATDLVGYVKDMIAKGATQVYVFNLPDSSLTPDGVASGTTGQALLHALVGTFNTTLQSGLAGTSARIIDFNAQLTAAIQNGASFGFANTSARACDATKINALVPSAGGSSLFCSANTLVASGADQSYLFADGVHPTTAGHRLIASNVLARLLADNVAH

SEQ ID NO: 9 - protein, 261 aa, Streptomyces coelicolor
MIGSYVAVGDSFTEGVGDPGPDGAFVGWADRLAVLLADRRPEGDFTYTNLAVRGRLLDQIVAEQVPRVVGLAPDLVSFAAGGNDIIRPGTDPDEVAERFELAVAALTAAAGTVLVTTGFDTRGVPVLKHLRGKIATYNGHVRAIADRYGCPVLDLWSLRSVQDRRAWDADRLHLSPEGHTRVALRAGQALGLRVPADPDQPWPPLPPRGTLDVRRDDVHWAREYLVPWIGRRLRGESSGDHVTAKGTLSPDAIKTRIAAVA

SEQ ID NO: 10 - protein, 260 aa, Streptomyces coelicolor
MQTNPAYTSLVAVGDSFTEGMSDLLPDGSYRGWADLLATRMAARSPGFRYANLAVRGKLIGQIVDEQVDVAAAMGADVITLVGGLNDTLRPKCDMARVRDLLTQAVERLAPHCEQLVLMRSPGRQGPVLERFRPRMEALFAVIDDLAGRHGAVVVDLYGAQSLADPRMWDVDRLHLTAEGHRRVAEAVWQSLGHEPEDPEWHAPIPATPPPGWVTRRTADVRFARQHLLPWIGRRLTGRSSGDGLPAKRPDLLPYEDPAR

SEQ ID NO: 11 - protein, 454 aa, Streptomyces coelicolor
MTRGRDGGAGAPPTKHRALLAAIVTLIVAISAAIYAGASADDGSRDHALQAGGRLPRGDAAPASTGAWVGAWATAPAAAEPGTETTGLAGRSVRNVVHTSVGGTGARITLSNLYGQSPLTVTHASIALAAGPDTAAAIADTMRRLTFGGSARVIIPAGGQVMSDTARLAIPYGANVLVTTYSPIPSGPVTYHPQARQTSYLADGDRTADVTAVAYTTPTPYWRYLTALDVLSHEADGTVVAFGDSITDGARSQSDANHRWTDVLAARLHEAAGDGRDTPRYSVVNEGISGNRLLTSRPGRPADNPSGLSRFQRDVLERTNVKAVVVVLGVNDVLNSPELADRDAILTGLRTLVDRAHARGLRVVGATITPFGGYGGYTEARETMRQEVNEEIRSGRVFDTVVDFDKALRDPYDPRRMRSDYDSGDHLHPGDKGYARMGAVIDLAALKGAAPVKA

SEQ ID NO: 12 - protein, 340 aa, Streptomyces coelicolor
MTSMSRARVARRIAAGAAYGGGGIGLAGAAAVGLVVAEVQLARRRVGVGTPTRVPNAQGLYGGTLPTAGDPPLRLMMLGDSTAAGQGVHRAGQTPGALLASGLAAVAERPVRLGSVAQPGACSDDLDRQVALVLAEPDRVPDICVIMVGANDVTHRMPATRSVRHLSSAVRRLRTAGAEVVVGTCPDLGTIERVRQPLRWLARRASRQLAAAQTIGAVEQGGRTVSLGDLLGPEFAQNPRELFGPDNYHPSAEGYATAAMAVLPSVCAALGLWPADEEHPDALRREGFLPVARAAAEAASEAGTEVAAAMPTGPRGPWALLKRRRRRRVSEAEPSSPSGV

SEQ ID NO: 13 - protein, 305 aa, Streptomyces coelicolor
MGRGTDQRTRYGRRRARVALAALTAAVLGVGVAGCDSVGGDSPAPSGSPSKRTRTAPAWDTSPASVAAVGDSITRGFDACAVLSDCPEVSWATGSSAKVDSLAVRLLGKADAAEHSWNYAVTGARMADLTAQVTRAAQREPELVAVMAGANDACRSTTSAMTPVADFRAQFEEAMATLRKKLPKAQVYVSSIPDLKRLWSQGRTNPLGKQVWKLGLCPSMLGDADSLDSAATLRRNTVRDRVADYNEVLREVCAKDRRCRSDDGAVHEFRFGTDQLSHWDWFHPSVDGQARLAEIAYRAVTAKNP

SEQ ID NO: 14 - protein, 268 aa, Streptomyces rimosus
MRLSRRAATASALLLTPALALFGASAAVSAPRIQATDYVALGDSYSSGVGAGSYDSSSGSCKRSTKSYPALWAASHTGTRFNFTACSGARTGDVLAKQLTPVNSGTDLVSITIGGNDAGFADTMTTCNLQGESACLARIAKARAYIQQTLPAQLDQVYDAIDSRAPAAQVVVLGYPRFYKLGGSCAVGLSEKSRAAINAAADDINAVTAKRAADHGFAFGDVNTTFAGHELCSGAPWLHSVTLPVENSYHPTANGQSKGYLPVLNSAT

SEQ ID NO: 15 - protein, 336 aa, Aeromonas salmonicida subsp. Salmonicida; identical to SEQ ID NO: 4

SEQ ID NO: 16 - protein, 318 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIETQYEFLAHG

SEQ ID NO: 17 - protein, 465 aa, Candida parapsilosis
MRYFAIAFLLINTISAFVLAPKKPSQDDFYTPPQGYEAQPLGSILKTRNVPNPLTNVFTPVKVQNAWQLLVRSEDTFGNPNAIVTTIIQPFNAKKDKLVSYQTFEDSGKLDCAPSYAIQYGSDISTLTTQGEMYYISALLDQGYYVVTPDYEGPKSTFTVGLQSGRATLNSLRATLKSGNLTGVSSDAETLLWGYSGGSLASGWAAAIQKEYAPELSKNLLGAALGGFVTNITATAEAVDSGPFAGIISNALAGIGNEYPDFKNYLLKKVSPLLSITYRLGNTHCLLDGGIAYFGKSFFSRIIRYFPDGWDLVNQEPIKTILQDNGLVYQPKDLTPQIPLFIYHGTLDAIVPIVNSRKTFQQWCDWGLKSGEYNEDLTNGHITESIVGAPAALTWIINRFNGQPPVDGCQHNVRASNLEYPGTPQSIKNYFEAALHAILGFDLGPDVKRDKVTLGGLLKLERFAF

SEQ ID NO: 18 - protein, 471 aa, Candida parapsilosis
MRYFAIAFLLINTISAFVLAPKKPSQDDFYTPPQGYEAQPLGSILKTRNVPNPLTNVFTPVKVQNAWQLLVRSEDTFGNPNAIVTTIIQPFNAKKDKLVSYQTFEDSGKLDCAPSYAIQYGSDISTLTTQGEMYYISALLDQGYYVVTPDYEGPKSTFTVGLQSGRATLNSLRATLKSGNLTGVSSDAETLLWGYSGGSLASGWAAAIQKEYAPELSKNLLGAALGGFVTNITATAEAVDSGPFAGIISNALAGIGNEYPDFKNYLLKKVSPLLSITYRLGNTHCLLDGGIAYFGKSFFSRIIRYFPDGWDLVNQEPIKTILQDNGLVYQPKDLTPQIPLFIYHGTLDAIVPIVNSRKTFQQWCDWGLKSGEYNEDLTNGHITESIVGAPAALTWIINRFNGQPPVDGCQHNVRASNLEYPGTPQSIKNYFEAALHAILGFDLGPDVKRDKVTLGGLLKLERFAFHHHHHH

SEQ ID NO: 19 - protein, 261 aa, Streptomyces coelicolor; identical to SEQ ID NO: 9

SEQ ID NO: 20 - protein, 4 aa, Artificial Sequence
  Peptide motif
  misc_feature (4)..(4): Xaa can be any naturally occurring amino acid
GDSX

SEQ ID NO: 21 - protein, 18 aa, Aeromonas sp.
MKKWFVCLLGLIALTVQA

SEQ ID NO: 22 - protein, 29 aa, Bacillus subtilis
MRSKKLWISLLFALTLIFTMAFSNMSAQA

SEQ ID NO: 23 - protein, 29 aa, Bacillus licheniformis
MMRKKSFWFGMLTAFMLVFTMEFSDSASA

SEQ ID NO: 24 - protein, 5 aa, Artificial Sequence
  Peptide motif
GANDY

SEQ ID NO: 25 - protein, 347 aa, Artificial Sequence
  Fusion construct
MFKFKKNFLVGLSAALMSISLFSATASAASADSRPAFSRIVMFGDSLSDTGKMYSKMRGYLPSSPPYYEGRFSNGPVWLEQLTKQFPGLTIANEAEGGATAVAYNKISWNPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNQLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIANQYEFLAH

SEQ ID NO: 26 - protein, 267 aa, Streptomyces sp.
MRLTRSLSAASVIVFALLLALLGISPAQAAGPAYVALGDSYSSGNGAGSYIDSSGDCHRSNNAYPARWAAANAPSSFTFAACSGAVTTDVINNQLGALNASTGLVSITIGGNDAGFADAMTTCVTSSDSTCLNRLATATNYINTTLLARLDAVYSQIKARAPNARVVVLGYPRMYLASNPWYCLGLSNTKRAAINTTADTLNSVISSRATAHGFRFGDVRPTFNNHELFFGNDWLHSLTLPVWESYHPTSTGHQSGYLPVLNANSST

SEQ ID NO: 27 - protein, 548 aa, Thermobifida sp.
MLPHPAGERGEVGAFFALLVGTPQDRRLRLECHETRPLRGRCGCGERRVPPLTLPGDGVLCTTSSTRDAETVWRKHLQPRPDGGFRPHLGVGCLLAGQGSPGVLWCGREGCRFEVCRRDTPGLSRTRNGDSSPPFRAGWSLPPKCGEISQSARKTPAVPRYSLLRTDRPDGPRGRFVGSGPRAATRRRLFLGIPALVLVTALTLVLAVPTGRETLWRMWCEATQDWCLGVPVDSRGQPAEDGEFLLLSPVQAATWGNYYALGDSYSSGDGARDYYPGTAVKGGCWRSANAYPELVAEAYDFAGHLSFLACSGQRGYAMLDAIDEVGSQLDWNSPHTSLVTIGIGGNDLGFSTVLKTCMVRVPLLDSKACTDQEDAIRKRMAKFETTFEELISEVRTRAPDARILVVGYPRIFPEEPTGAYYTLTASNQRWLNETIQEFNQQLAEAVAVHDEEIAASGGVGSVEFVDVYHALDGHEIGSDEPWVNGVQLRDLATGVTVDRSTFHPNAAGHRAVGERVIEQIETGPGRPLYATFAVVAGATVDTLAGEVG

SEQ ID NO: 28 - protein, 372 aa, Thermobifida sp.
MGSGPRAATRRRLFLGIPALVLVTALTLVLAVPTGRETLWRMWCEATQDWCLGVPVDSRGQPAEDGEFLLLSPVQAATWGNYYALGDSYSSGDGARDYYPGTAVKGGCWRSANAYPELVAEAYDFAGHLSFLACSGQRGYAMLDAIDEVGSQLDWNSPHTSLVTIGIGGNDLGFSTVLKTCMVRVPLLDSKACTDQEDAIRKRMAKFETTFEELISEVRTRAPDARILVVGYPRIFPEEPTGAYYTLTASNQRWLNETIQEFNQQLAEAVAVHDEEIAASGGVGSVEFVDVYHALDGHEIGSDEPWVNGVQLRDLATGVTVDRSTFHPNAAGHRAVGERVIEQIETGPGRPLYATFAVVAGATVDTLAGEVG

SEQ ID NO: 29 - protein, 300 aa, Corynebacterium efficiens
MRTTVIAASALLLLAGCADGAREETAGAPPGESSGGIREEGAEASTSITDVYIALGDSYAAMGGRDQPLRGEPFCLRSSGNYPELLHAEVTDLTCQGAVTGDLLEPRTLGERTLPAQVDALTEDTTLVTLSIGGNDLGFGEVAGCIRERIAGENADDCVDLLGETIGEQLDQLPPQLDRVHEAIRDRAGDAQVVVTGYLPLVSAGDCPELGDVSEADRRWAVELTGQINETVREAAERHDALFVLPDDADEHTSCAPPQQRWADIQGQQTDAYPLHPTSAGHEAMAAAVRDALGLEPVQP

SEQ ID NO: 30 - protein, 284 aa, Novosphingobium aromaticivorans
MGQVKLFARRCAPVLLALAGLAPAATVAREAPLAEGARYVALGSSFAAGPGVGPNAPGSPERCGRGTLNYPHLLAEALKLDLVDATCSGATTHHVLGPWNEVPPQIDSVNGDTRLVTLTIGGNDVSFVGNIFAAACEKMASPDPRCGKWREITEEEWQADEERMRSIVRQIHARAPLARVVVVDYITVLPPSGTCAAMAISPDRLAQSRSAAKRLARITARVAREEGASLLKFSHISRRHHPCSAKPWSNGLSAPADDGIPVHPNRLGHAEAAAALVKLVKLMK

SEQ ID NO: 31 - protein, 268 aa, Streptomyces coelicolor
MRRFRLVGFLSSLVLAAGAALTGAATAQAAQPAAADGYVALGDSYSSGVGAGSYISSSGDCKRSTKAHPYLWAAAHSPSTFDFTACSGARTGDVLSGQLGPLSSGTGLVSISIGGNDAGFADTMTTCVLQSESSCLSRIATAEAYVDSTLPGKLDGVYSAISDKAPNAHVVVIGYPRFYKLGTTCIGLSETKRTAINKASDHLNTVLAQRAAAHGFTFGDVRTTFTGHELCSGSPWLHSVNWLNIGESYHPTAAGQSGGYLPVLNGAA

SEQ ID NO: 32 - protein, 269 aa, Streptomyces avermitilis
MRRSRITAYVTSLLLAVGCALTGAATAQASPAAAATGYVALGDSYSSGVGAGSYLSSSGDCKRSSKAYPYLWQAAHSPSSFSFMACSGARTGDVLANQLGTLNSSTGLVSLTIGGNDAGFSDVMTTCVLQSDSACLSRINTAKAYVDSTLPGQLDSVYTAISTKAPSAHVAVLGYPRFYKLGGSCLAGLSETKRSAINDAADYLNSAIAKRAADHGFTFGDVKSTFTGHEICSSSTWLHSLDLLNIGQSYHPTAAGQSGGYLPVMNSVA

SEQ ID NO: 33 - protein, 267 aa, Streptomyces sp.; identical to SEQ ID NO: 26

SEQ ID NO: 34 - protein, 317 aa, Aeromonas hydrophila
ADSRPAFSRIVMFGDSLSDTGKMYSKMRGYLPSSPPYYEGRFSNGPVWLEQLTKQFPGLTIANEAEGGATAVAYNKISWNPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNQLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIANQYEFLAH

SEQ ID NO: 35 - protein, 318 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWNPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIETQYEFLAHG

SEQ ID NO: 36 - DNA, 1371 bp, Streptomyces thermosacchari
acaggccgatgcacggaaccgtacctttccgcagtgaagcgctctccccccatcgttcgccgggacttcatccgcgattttggcatgaacacttccttcaacgcgcgtagcttgctacaagtgcggcagcagacccgctcgttggaggctcagtgagattgacccgatccctgtcggccgcatccgtcatcgtcttcgccctgctgctcgcgctgctgggcatcagcccggcccaggcagccggcccggcctatgtggccctgggggattcctattcctcgggcaacggcgccggaagttacatcgattcgagcggtgactgtcaccgcagcaacaacgcgtaccccgcccgctgggcggcggccaacgcaccgtcctccttcaccttcgcggcctgctcgggagcggtgaccacggatgtgatcaacaatcagctgggcgccctcaacgcgtccaccggcctggtgagcatcaccatcggcggcaatgacgcgggcttcgcggacgcgatgaccacctgcgtcaccagctcggacagcacctgcctcaaccggctggccaccgccaccaactacatcaacaccaccctgctcgcccggctcgacgcggtctacagccagatcaaggcccgtgcccccaacgcccgcgtggtcgtcctcggctacccgcgcatgtacctggcctcgaacccctggtactgcctgggcctgagcaacaccaagcgcgcggccatcaacaccaccgccgacaccctcaactcggtgatctcctcccgggccaccgcccacggattccgattcggcgatgtccgcccgaccttcaacaaccacgaactgttcttcggcaacgactggctgcactcactcaccctgccggtgtgggagtcgtaccaccccaccagcacgggccatcagagcggctatctgccggtcctcaacgccaacagctcgacctgatcaacgcacggccgtgcccgccccgcgcgtcacgctcggcgcgggcgccgcagcgcgttgatcagcccacagtgccggtgacggtcccaccgtcacggtcgagggtgtacgtcacggtggcgccgctccagaagtggaacgtcagcaggaccgtggagccgtccctgacctcgtcgaagaactccggggtcagcgtgatcacccctcccccgtagccgggggcgaaggcggcgccgaactccttgtaggacgtccagtcgtgcggcccggcgttgccaccgtccgcgtagaccgcttccatggtcgccagccggtccccgcggaactcggtggggatgtccgtgcccaaggtggtcccggtggtgtccgagagcaccgggggctcgtaccggatgatgtgcagatccaaagaatt

SEQ ID NO: 37 - protein, 267 aa, Streptomyces thermosacchari; identical to SEQ ID NO: 26

SEQ ID NO: 38 - protein, 548 aa, Thermobifida fusca; identical to SEQ ID NO: 27

SEQ ID NO: 39 - DNA, 3000 bp, Thermobifida fusca
ggtggtgaaccagaacacccggtcgtcggcgtgggcgtccaggtgcaggtgcaggttcttcaactgctccagcaggatgccgccgtggccgtgcacgatggccttgggcaggcctgtggtccccgacgagtacagcacccatagcggatggtcgaacggcagcggggtgaactccagttccgcgccttcgcccgcggcttcgaactccgcccaggacagggtgtcggcgacagggccgcagcccaggtacggcaggacgacggtgtgctgcaggctgggcatgccgtcgcgcagggctttgagcacgtcacggcggtcgaagtccttaccgccgtagcggtagccgtccacggccagcagcactttcggttcgatctgcgcgaaccggtcgaggacgctgcgcaccccgaagtcgggggaacaggacgaccaggtcgcaccgatcgcggcgcaggcgaggaatgcggccgtcgcctcggcgatgttcggcaggtaggccacgacccggtcgccggggcccaccccgaggctgcggagggccgcagcgatcgcggcggtgcgggtccgcagttctccccaggtccactcggtcaacggccggagttcggacgcgtgccggatcgccacggctgatgggtcacggtcgcggaagatgtgctcggcgtagttgagggtggcgccggggaaccagacggcgccgggcatggcgtcggaggcgagcactgtggtgtacggggtggcggcgcgcacccggtagtactcccagatcgcggaccagaatccttcgaggtcggttaccgaccagcgccacagtgcctcgtagtccggtgcgtccacaccgcggtgctcccgcacccagcgggtgaacgcggtgaggttggcgcgttctttgcgctcctcgtcgggactccacaggatcggcggctgcggcttgagtgtcatgaaacgcgaccccttcgtggacggtgcggatgcggtgagcgtcgggtgcctcccctaacgctccccggtgacggagtgttgtgcaccacatctagcacgcgggacgcggaaaccgtatggagaaaacacctacaaccccggccggacggtgggtttcggccacacttaggggtcgggtgcctgcttgccgggcagggcagtcccggggtgctgtggtgcgggcgggagggctgtcgcttcgaggtgtgccggcgggacactccgggcctcagccgtacccgcaacggggacagttctcctcccttccgggctggatggtcccttcccccgaaatgcggcgagatctcccagtcagcccggaaaacacccgctgtgcccaggtactctttgcttcgaacagacaggccggacggtccacgggggaggtttgtgggcagcggaccacgtgcggcgaccagacgacggttgttcctcggtatccccgctcttgtacttgtgacagcgctcacgctggtcttggctgtcccgacggggcgcgagacgctgtggcgcatgtggtgtgaggccacccaggactggtgcctgggggtgccggtcgactcccgcggacagcctgcggaggacggcgagtttctgctgctttctccggtccaggcagcgacctgggggaactattacgcgctcggggattcgtactcttcgggggacggggcccgcgactactatcccggcaccgcggtgaagggcggttgctggcggtccgctaacgcctatccggagctggtcgccgaagcctacgacttcgccggacacttgtcgttcctggcctgcagcggccagcgcggctacgccatgcttgacgctatcgacgaggtcggctcgcagctggactggaactcccctcacacgtcgctggtgacgatcgggatcggcggcaacgatctggggttctccacggttttgaagacctgcatggtgcgggtgccgctgctggacagcaaggcgtgcacggaccaggaggacgctatccgcaagcggatggcgaaattcgagacgacgtttgaagagctcatcagcgaagtgcgcacccgcgcgccggacgcccggatccttgtcgtgggctacccccggatttttccggaggaaccgaccggcgcctactacacgctgaccgcgagcaaccagcggtggctcaacgaaaccattcaggagttcaaccagcagctcgccgaggctgtcgcggtccacgacgaggagattgccgcgtcgggcggggtgggcagcgtggagttcgtggacgtctaccacgcgttggacggccacgagatcggctcggacgagccgtgggtgaacggggtgcagttgcgggacctcgccaccggggtgactgtggaccgcagtaccttccaccccaacgccgctgggcaccgggcggtcggtgagcgggtcatcgagcagatcgaaaccggcccgggccgtccgctctatgccactttcgcggtggtggcgggggcgaccgtggacactctcgcgggcgaggtggggtgacccggcttaccgtccggcccgcaggtctgcgagcactgcggcgatctggtccactgcccagtgcagttcgtcttcggtgatgaccagcggcggggagagccggatcgttgagccgtgcgtgtctttgacgagcacaccccgctgcaggagccgttcgcacagttctcttccggtggccagagtcgggtcgacgtcgatcccagcccacaggccgatgctgcgggccgcgaccacgccgttgccgaccagttggtcgaggcgggcgcgcagcacgggggcgagggcgcggacatggtccaggtaagggccgtcgcggacgaggctcaccacggcagtgccgaccgcgcaggcgagggcgttgccgccgaaggtgctgccgtgctggccggggcggatcacgtcgaagacttccgcgtcgcctaccgccgccgccacgggcaggatgccgccgcccagcgctttgccgaacaggtagatatcggcgtcgactccgctgtggtcgcaggcccg

SEQ ID NO: 40 - protein, 372 aa, Thermobifida fusca
VGSGPRAATRRRLFLGIPALVLVTALTLVLAVPTGRETLWRMWCEATQDWCLGVPVDSRGQPAEDGEFLLLSPVQAATWGNYYALGDSYSSGDGARDYYPGTAVKGGCWRSANAYPELVAEAYDFAGHLSFLACSGQRGYAMLDAIDEVGSQLDWNSPHTSLVTIGIGGNDLGFSTVLKTCMVRVPLLDSKACTDQEDAIRKRMAKFETTFEELISEVRTRAPDARILVVGYPRIFPEEPTGAYYTLTASNQRWLNETIQEFNQQLAEAVAVHDEEIAASGGVGSVEFVDVYHALDGHEIGSDEPWVNGVQLRDLATGVTVDRSTFHPNAAGHRAVGERVIEQIETGPGRPLYATFAVVAGATVDTLAGEVG

SEQ ID NO: 41 - protein, 300 aa, Corynebacterium efficiens; identical to SEQ ID NO: 29

SEQ ID NO: 42 - DNA, 3000 bp, Corynebacterium efficiens
ttctggggtgttatggggttgttatcggctcgtcctgggtggatcccgccaggtggggtattcacgggggacttttgtgtccaacagccgagaatgagtgccctgagcggtgggaatgaggtgggcggggctgtgtcgccatgagggggcggcgggctctgtggtgccccgcgacccccggccccggtgagcggtgaatgaaatccggctgtaatcagcatcccgtgcccaccccgtcggggaggtcagcgcccggagtgtctacgcagtcggatcctctcggactcggccatgctgtcggcagcatcgcgctcccgggtcttggcgtccctcggctgttctgcctgctgtccctggaaggcgaaatgatcaccggggagtgatacaccggtggtctcatcccggatgcccacttcggcgccatccggcaattcgggcagctccgggtggaagtaggtggcatccgatgcgtcggtgacgccatagtgggcgaagatctcatcctgctcgagggtgctcaggccactctccggatcgatatcgggggcgtccttgatggcgtccttgctgaaaccgaggtgcagcttgtgggcttccaatttcgcaccacggagcgggacgaggctggaatgacggccgaagagcccgtggtggacctcaacgaaggtgggtagtcccgtgtcatcattgaggaacacgccctccaccgcacccagcttgtggccggagttgtcgtaggcgctggcatccagaagggaaacgatctcatatttgtcggtgtgctcagacatgatcttcctttgctgtcggtgtctggtactaccacggtagggctgaatgcaactgttatttttctgttattttaggaattggtccatatcccacaggctggctgtggtcaaatcgtcatcaagtaatccctgtcacacaaaatgggtggtgggagccctggtcgcggttccgtgggaggcgccgtgccccgcaggatcgtcggcatcggcggatctggccggtaccccgcggtgaataaaatcattctgtaaccttcatcacggttggttttaggtatccgcccctttcgtcctgaccccgtccccggcgcgcgggagcccgcgggttgcggtagacaggggagacgtggacaccatgaggacaacggtcatcgcagcaagcgcattactccttctcgccggatgcgcggatggggcccgggaggagaccgccggtgcaccgccgggtgagtcctccgggggcatccgggaggagggggcggaggcgtcgacaagcatcaccgacgtctacatcgccctcggggattcctatgcggcgatgggcgggcgggatcagccgttacggggtgagccgttctgcctgcgctcgtccggtaattacccggaactcctccacgcagaggtcaccgatctcacctgccagggggcggtgaccggggatctgctcgaacccaggacgctgggggagcgcacgctgccggcgcaggtggatgcgctgacggaggacaccaccctggtcaccctctccatcgggggcaatgacctcggattcggggaggtggcgggatgcatccgggaacggatcgccggggagaacgctgatgattgcgtggacctgctgggggaaaccatcggggagcagctcgatcagcttcccccgcagctggaccgcgtgcacgaggctatccgggaccgcgccggggacgcgcaggttgtggtcaccggttacctgccgctcgtgtctgccggggactgccccgaactgggggatgtctccgaggcggatcgtcgttgggcggttgagctgaccgggcagatcaacgagaccgtgcgcgaggcggccgaacgacacgatgccctctttgtcctgcccgacgatgccgatgagcacaccagttgtgcacccccacagcagcgctgggcggatatccagggccaacagaccgatgcctatccgctgcacccgacctccgccggccatgaggcgatggccgccgccgtccgggacgcgctgggcctggaaccggtccagccgtagcgccgggcgcgcgcttgtcgacgaccaacccatgccaggctgcagtcacatccgcacatagcgcgcgcgggcgatggagtacgcaccatagaggatgagcccgatgccgacgatgatgagcagcacactgccgaagggttgttccccgagggtgcgcagagccgagtccagacctgcggcctgctccggatcatgggcccaaccggcgatgacgatcaacacccccaggatcccgaaggcgataccacgggcgacataaccggctgttccggtgatgatgatcgcggtcccgacctgccctgaccccgcacccgcctccagatcctcccggaaatcccgggtggcccccttccagaggttgtagacacccgcccccagtaccaccagcccggcgaccacaaccagcaccacaccccagggttgggataggacggtggcggtgacatcggtggcggtctccccatcggaggtgctgccgccccgggcgaaggtggaggtggtcaccgccagggagaagtagaccatggccatgaccgcccccttggccctttccttgaggtcctcgcccgccagcagctggctcaattgccagagtcccagggccgccagggcgatgacggcaacccacaggaggaactgcccacccggagcctccgcgatggtggccagggcacctgaattcgaggcctcatcacccgaaccgccggatccagtggcgatgcgcaccgcgatccacccgatgaggatgtgcagtatgcccaggacaatgaaaccacctctggccagggtggtcagcgcggggtggtcctcggcctggtcggcagcccgttcgatcgtccgtttcgcggatctggtgtcgcccttatccatagctcccattgaaccgccttgaggggtgggcggccactgtcagggcggattgtgatctgaactgtgatgttccatcaaccc

SEQ ID NO: 43 - protein, 268 aa, Streptomyces coelicolor; identical to SEQ ID NO: 31

SEQ ID NO: 44 - DNA, 2000 bp, Streptomyces coelicolor
cccggcggcccgtgcaggagcagcagccggcccgcgatgtcctcgggcgtcgtcttcatcaggccgtccatcgcgtcggcgaccggcgccgtgtagttggcccggacctcgtcccaggtgcccgcggcgatctggcgggtggtgcggtgcgggccgcgccgaggggagacgtaccagaagcccatcgtcacgttctccggctgcggttcgggctcgtccgccgctccgtccgtcgcctcgccgagcaccttctcggcgaggtcggcgctggtcgccgtcaccgtgacgtcggcgccccggctccagcgcgagatcagcagcgtccagccgtcgccctccgccagcgtcgcgctgcggtcgtcgtcgcgggcgatccgcagcacgcgcgcgccgggcggcagcagcgtggcgccggaccgtacgcggtcgatgttcgccgcgtgcgagtacggctgctcacccgtggcgaaacggccgaggaacagcgcgtcgacgacgtcggacggggagtcgctgtcgtccacgttgagccggatcggcagggcttcgtgcgggttcacggacatgtcgccatgatcgggcacccggccgccgcgtgcacccgctttcccgggcacgcacgacaggggctttctcgccgtcttccgtccgaacttgaacgagtgtcagccatttcttggcatggacacttccagtcaacgcgcgtagctgctaccacggttgtggcagcaatcctgctaagggaggttccatgagacgtttccgacttgtcggcttcctgagttcgctcgtcctcgccgccggcgccgccctcaccggggcagcgaccgcccaggcggcccaacccgccgccgccgacggctatgtggccctcggcgactcctactcctccggggtcggagcgggcagctacatcagctcgagcggcgactgcaagcgcagcacgaaggcccatccctacctgtgggcggccgcccactcgccctccacgttcgacttcaccgcctgttccggcgcccgtacgggtgatgttctctccggacagctcggcccgctcagctccggcaccggcctcgtctcgatcagcatcggcggcaacgacgccggtttcgccgacaccatgacgacctgtgtgctccagtccgagagctcctgcctgtcgcggatcgccaccgccgaggcgtacgtcgactcgacgctgcccggcaagctcgacggcgtctactcggcaatcagcgacaaggcgccgaacgcccacgtcgtcgtcatcggctacccgcgcttctacaagctcggcaccacctgcatcggcctgtccgagaccaagcggacggcgatcaacaaggcctccgaccacctcaacaccgtcctcgcccagcgcgccgccgcccacggcttcaccttcggcgacgtacgcaccaccttcaccggccacgagctgtgctccggcagcccctggctgcacagcgtcaactggctgaacatcggcgagtcgtaccaccccaccgcggccggccagtccggtggctacctgccggtcctcaacggcgccgcctgacctcaggcggaaggagaagaagaaggagcggagggagacgaggagtgggaggccccgcccgacggggtccccgtccccgtctccgtctccgtcccggtcccgcaagtcaccgagaacgccaccgcgtcggacgtggcccgcaccggactccgcacctccacgcgcacggcactctcgaacgcgccggtgtcgtcgtgcgtcgtcaccaccacgccgtcctggcgcgagcgctcgccgcccgacgggaaggacagcgtccgccaccccggatcggagaccgacccgtccgcggtcacccaccggtagccgacctccgcgggcagccgcccgaccgtgaacgtcgccgtgaacgcgggtgcccggtcgtgcggcggcggacaggcccccgagtagtgggtgcgcgagcccaccacggtcacctccaccgactgcgctgcggggc

SEQ ID NO: 45 - protein, 269 aa, Streptomyces avermitilis; identical to SEQ ID NO: 32

SEQ ID NO: 46 - DNA, 1980 bp, Streptomyces avermitilis
ccaccgccgggtcggcggcgagtctcctggcctcggtcgcggagaggttggccgtgtagccgttcagcgcggcgccgaacgtcttcttcaccgtgccgccgtactcgttgatcaggcccttgcccttgctcgacgcggccttgaagccggtgcccttcttgagcgtgacgatgtagctgcccttgatcgcggtgggggagccggcggcgagcaccgtgccctcggccggggtggcctgggcgggcagtgcggtgaatccgcccacgagggcgccggtcgccacggcggttatcgcggcgatccggatcttcttgctacgcagctgtgccatacgagggagtcctcctctgggcagcggcgcgcctgggtggggcgcacggctgtggggggtgcgcgcgtcatcacgcacacggccctggagcgtcgtgttccgccctgggttgagtaaagcctcggccatctacgggggtggctcaagggagttgagaccctgtcatgagtctgacatgagcacgcaatcaacggggccgtgagcaccccggggcgaccccggaaagtgccgagaagtcttggcatggacacttcctgtcaacacgcgtagctggtacgacggttacggcagagatcctgctaaagggaggttccatgagacgttcccgaattacggcatacgtgacctcactcctcctcgccgtcggctgcgccctcaccggggcagcgacggcgcaggcgtccccagccgccgcggccacgggctatgtggccctcggcgactcgtactcgtccggtgtcggcgccggcagctacctcagctccagcggcgactgcaagcgcagttcgaaggcctatccgtacctctggcaggccgcgcattcaccctcgtcgttcagtttcatggcttgctcgggcgctcgtacgggtgatgtcctggccaatcagctcggcaccctgaactcgtccaccggcctggtctccctcaccatcggaggcaacgacgcgggcttctccgacgtcatgacgacctgtgtgctccagtccgacagcgcctgcctctcccgcatcaacacggcgaaggcgtacgtcgactccaccctgcccggccaactcgacagcgtgtacacggcgatcagcacgaaggccccgtcggcccatgtggccgtgctgggctacccccgcttctacaaactgggcggctcctgcctcgcgggcctctcggagaccaagcggtccgccatcaacgacgcggccgactatctgaacagcgccatcgccaagcgcgccgccgaccacggcttcaccttcggcgacgtcaagagcaccttcaccggccatgagatctgctccagcagcacctggctgcacagtctcgacctgctgaacatcggccagtcctaccacccgaccgcggccggccagtccggcggctatctgccggtcatgaacagcgtggcctgagctcccacggcctgaatttttaaggcctgaatttttaaggcgaaggtgaaccggaagcggaggccccgtccgtcggggtctccgtcgcacaggtcaccgagaacggcacggagttggacgtcgtgcgcaccgggtcgcgcacctcgacggcgatctcgttcgagatcgttccgctcgtgtcgtacgtggtgacgaacacctgcttctgctgggtctttccgccgctcgccgggaaggacagcgtcttccagcccggatccgggacctcgcccttcttggtcacccagcggtactccacctcgaccggcacccggcccaccgtgaaggtcgccgtgaacgtgggcgcctgggcggtgggcggcgggcaggcaccggagtagtcggtgtgcacgccggtgaccgtcaccttcacggactgggccggcggggtcgtcgtaccgccgccgccaccgccgcctcccggagtggagcccgagctgtggtcgcccccgccgtcggcgttgtcgtcctcgggggttttcgaac

SEQ ID NO: 47 - protein, 372 aa, Thermobifida fusca; identical to SEQ ID NO: 28

SEQ ID NO: 48 - DNA, 968 bp, Thermobifida fusca
ctgcagacacccgccccgccttctcccggatcgtcatgttcggcgactccctcagcgacaccggcaagatgtactccaagatgcgcggctacctgccgtcctccccgccgtactacgagggccgcttctcgaacggcccggtctggctggagcagctgacgaagcagttccccggcctgacgatcgccaacgaggccgaggggggcgcgaccgcagtcgcctacaacaagatctcctggaacccgaagtaccaggtcattaacaacctcgactacgaggtcacccagttcttgcagaaggactcgttcaagcccgacgacctggtcatcctgtgggtgggcgccaacgactacctggcctacggttggaacacggagcaggacgccaagcgggtgcgcgacgccatctcggacgcggcaaaccgcatggtcctgaacggcgcgaagcagatcctgctgttcaacctgcccgacctgggccagaacccgtccgcccgctcccagaaggtcgtcgaggccgtctcgcacgtgtccgcctaccacaacaagctgctcctcaacctcgcccggcagctcgccccgacgggcatggtcaagctgttcgagatcgacaagcagttcgcggagatgctgcgcgacccccagaacttcggcctgagcgacgtggagaacccgtgctacgacggcggctacgtgtggaagccgttcgccacccggtccgtctcgaccgaccggcagctgtcggccttctcgccccaggagcgcctggcgatcgctggcaacccgctcctggcacaggcggtagcttcgccgatggcccgccgctcggcctcgcccctcaactgcgagggcaagatgttctgggaccaggtccaccccaccaccgtggtccacgccgccctctcggagcgcgccgccaccttcatcgagacccagtacgagttcctcgcccactagtctagaggatcc

SEQ ID NO: 49 - DNA, 1044 bp, Aeromonas salmonicida
atgaaacaacaaaaacggctttacgcccgattgctgacgctgttatttgcgctcatcttcttgctgcctcattctgcagcttcagcagcagatacaagaccggcgtttagccggatcgtcatgtttggagatagcctgagcgatacgggcaaaatgtatagcaaaatgagaggctatcttccgtcaagcccgccgtattatgaaggccgctttagcaatggaccggtctggctggaacaactgacgaaacaatttccgggactgacgatcgctaatgaagcagaaggaggagcaacagcggtcgcctataacaaaatcagctgggacccgaaatatcaggtcatcaacaacctggactatgaagtcacacagtttcttcagaaagacagctttaaaccggatgatctggtcatcctttgggtcggcgccaatgattatctggcgtatggctggaacacagaacaagatgccaaaagagtcagagatgccatcagcgatgccgctaatagaatggtcctgaacggcgccaaacaaatcctgctgtttaacctgccggatctgggacaaaatccgagcgccagaagccaaaaagtcgtcgaagcagtcagccatgtcagcgcctatcataacaaactgctgctgaacctggcaagacaattggcaccgacgggaatggttaaattgtttgaaattgacaaacagtttgccgaaatgctgagagatccgcaaaattttggcctgagcgatgtcgaaaacccgtgctatgatggcggatatgtctggaaaccgtttgccacaagaagcgtcagcacggatagacaactgtcagcgtttagcccgcaagaaagactggcaatcgccggaaatccgcttttggcacaagcagttgcttcaccgatggcaagaagatcagcaagcccgctgaattgcgaaggcaaaatgttttgggatcaggtccatccgacaacagttgtccatgctgccctttcagaaagagcggcgacgtttatcgaaacacagtatgaatttctggcccatggctga

SEQ ID NO: 50 - DNA, 1005 bp, Aeromonas hydrophila
atgaaaaaatggtttgtgtgtttattgggattggtcgcgctgacagttcaggcagccgacagccgtcccgccttctcccggatcgtgatgtttggcgacagcctctccgataccggcaagatgtacagcaagatgcgcggttacctcccctccagccccccctactatgagggccgcttctccaacgggcccgtctggctggagcagctgaccaacgagttcccgggcctgaccatagccaacgaggcggaaggcggaccgaccgccgtggcttacaacaagatctcctggaatcccaagtatcaggtcatcaacaacctggactacgaggtcacccagttcctgcaaaaagacagcttcaagccggacgatctggtgatcctctgggtcggcgccaacgactatctggcctatggctggaacacagagcaggatgccaagcgggtgcgcgacgccatcagcgatgcggccaaccgcatggtgctgaacggcgccaaggagatactgctgttcaacctgccggatctgggccagaacccctcggcccgcagccagaaggtggtcgaggcggccagccatgtctccgcctaccacaaccagctgctgctgaacctggcacgccagctggctcccaccggcatggtgaagctgttcgagatcgacaagcagtttgccgagatgctgcgtgatccgcagaacttcggcctgagcgaccagaggaacgcctgctacggtggcagctatgtatggaagccgtttgcctcccgcagcgccagcaccgacagccagctctccgccttcaacccgcaggagcgcctcgccatcgccggcaacccgctgctggcccaggccgtcgccagccccatggctgcccgcagcgccagcaccctcaactgtgagggcaagatgttctgggatcaggtccaccccaccactgtcgtgcacgccgccctgagcgagcccgccgccaccttcatcgagagccagtacgagttcctcgcccac

SEQ ID NO: 51 - DNA, 1011 bp, Aeromonas salmonicida
atgaaaaaatggtttgtttgtttattggggttgatcgcgctgacagttcaggcagccgacactcgccccgccttctcccggatcgtgatgttcggcgacagcctctccgataccggcaaaatgtacagcaagatgcgcggttacctcccctccagcccgccctactatgagggccgtttctccaacggacccgtctggctggagcagctgaccaagcagttcccgggtctgaccatcgccaacgaagcggaaggcggtgccactgccgtggcttacaacaagatctcctggaatcccaagtatcaggtctacaacaacctggactacgaggtcacccagttcttgcagaaagacagcttcaagccggacgatctggtgatcctctgggtcggtgccaatgactatctggcatatggctggaatacggagcaggatgccaagcgagttcgcgatgccatcagcgatgcggccaaccgcatggtactgaacggtgccaagcagatactgctgttcaacctgccggatctgggccagaacccgtcagcccgcagtcagaaggtggtcgaggcggtcagccatgtctccgcctatcacaacaagctgctgctgaacctggcacgccagctggcccccaccggcatggtaaagctgttcgagatcgacaagcaatttgccgagatgctgcgtgatccgcagaacttcggcctgagcgacgtcgagaacccctgctacgacggcggctatgtgtggaagccgtttgccacccgcagcgtcagcaccgaccgccagctctccgccttcagtccgcaggaacgcctcgccatcgccggcaacccgctgctggcacaggccgttgccagtcctatggcccgccgcagcgccagccccctcaactgtgagggcaagatgttctgggatcaggtacacccgaccactgtcgtgcacgcagccctgagcgagcgcgccgccaccttcatcgagacccagtacgagttcctcgcccacggatga

SEQ ID NO: 52 - DNA, 888 bp, Streptomyces coelicolor
atgccgaagcctgcccttcgccgtgtcatgaccgcgacagtcgccgccgtcggcacgctcgccctcggcctcaccgacgccaccgcccacgccgcgcccgcccaggccactccgaccctggactacgtcgccctcggcgacagctacagcgccggctccggcgtcctgcccgtcgaccccgccaacctgctctgtctgcgctcgacggccaactacccccacgtcatcgcggacacgacgggcgcccgcctcacggacgtcacctgcggcgccgcgcagaccgccgacttcacgcgggcccagtacccgggcgtcgcaccccagttggacgcgctcggcaccggcacggacctggtcacgctcaccatcggcggcaacgacaacagcaccttcatcaacgccatcacggcctgcggcacggcgggtgtcctcagcggcggcaagggcagcccctgcaaggacaggcacggcacctccttcgacgacgagatcgaggccaacacgtaccccgcgctcaaggaggcgctgctcggcgtccgcgccagggctccccacgccagggtggcggctctcggctacccgtggatcaccccggccaccgccgacccgtcctgcttcctgaagctccccctcgccgccggtgacgtgccctacctgcgggccatccaggcacacctcaacgacgcggtccggcgggccgccgaggagaccggagccacctacgtggacttctccggggtgtccgacggccacgacgcctgcgaggcccccggcacccgctggatcgaaccgctgctcttcgggcacagcctcgttcccgtccaccccaacgccctgggcgagcggcgcatggccgagcacacgatggacgtcctcggcctggactga

SEQ ID NO: 53 - DNA, 888 bp, Streptomyces coelicolor
tcagtccaggccgaggacgtccatcgtgtgctcggccatgcgccgctcgcccagggcgttggggtggacgggaacgaggctgtgcccgaagagcagcggttcgatccagcgggtgccgggggcctcgcaggcgtcgtggccgtcggacaccccggagaagtccacgtaggtggctccggtctcctcggcggcccgccggaccgcgtcgttgaggtgtgcctggatggcccgcaggtagggcacgtcaccggcggcgagggggagcttcaggaagcaggacgggtcggcggtggccggggtgatccacgggtagccgagagccgccaccctggcgtggggagccctggcgcggacgccgagcagcgcctccttgagcgcggggtacgtgttggcctcgatctcgtcgtcgaaggaggtgccgtgcctgtccttgcaggggctgcccttgccgccgctgaggacacccgccgtgccgcaggccgtgatggcgttgatgaaggtgctgttgtcgttgccgccgatggtgagcgtgaccaggtccgtgccggtgccgagcgcgtccaactggggtgcgacgcccgggtactgggcccgcgtgaagtcggcggtctgcgcggcgccgcaggtgacgtccgtgaggcgggcgcccgtcgtgtccgcgatgacgtgggggtagttggccgtcgagcgcagacagagcaggttggcggggtcgacgggcaggacgccggagccggcgctgtagctgtcgccgagggcgacgtagtccagggtcggagtggcctgggcgggcgcggcgtgggcggtggcgtcggtgaggccgagggcgagcgtgccgacggcggcgactgtcgcggtcatgacacggcgaagggcaggcttcggcat

SEQ ID NO: 54 - DNA, 717 bp, Saccharomyces cerevisiae
atggattacgagaagtttctgttatttggggattccattactgaatttgcttttaatactaggcccattgaagatggcaaagatcagtatgctcttggagccgcattagtcaacgaatatacgagaaaaatggatattcttcaaagagggttcaaagggtacacttctagatgggcgttgaaaatacttcctgagattttaaagcatgaatccaatattgtcatggccacaatatttttgggtgccaacgatgcatgctcagcaggtccccaaagtgtccccctccccgaatttatcgataatattcgtcaaatggtatctttgatgaagtcttaccatatccgtcctattataataggaccggggctagtagatagagagaagtgggaaaaagaaaaatctgaagaaatagctctcggatacttccgtaccaacgagaactttgccatttattccgatgccttagcaaaactagccaatgaggaaaaagttcccttcgtggctttgaataaggcgtttcaacaggaaggtggtgatgcttggcaacaactgctaacagatggactgcacttttccggaaaagggtacaaaatttttcatgacgaattattgaaggtcattgagacattctacccccaatatcatcccaaaaacatgcagtacaaactgaaagattggagagatgtgctagatgatggatctaacataatgtcttga

SEQ ID NO: 55 - DNA, 1044 bp, Ralstonia sp.
atgaacctgcgtcaatggatgggcgccgccacggctgcccttgccttgggcttggccgcgtgcgggggcggtgggaccgaccagagcggcaatcccaatgtcgccaaggtgcagcgcatggtggtgttcggcgacagcctgagcgatatcggcacctacacccccgtcgcgcaggcggtgggcggcggcaagttcaccaccaacccgggcccgatctgggccgagaccgtggccgcgcaactgggcgtgacgctcacgccggcggtgatgggctacgccacctccgtgcagaattgccccaaggccggctgcttcgactatgcgcagggcggctcgcgcgtgaccgatccgaacggcatcggccacaacggcggcgcgggggcgctgacctacccggttcagcagcagctcgccaacttctacgcggccagcaacaacacattcaacggcaataacgatgtcgtcttcgtgctggccggcagcaacgacattttcttctggaccactgcggcggccaccagcggctccggcgtgacgcccgccattgccacggcccaggtgcagcaggccgcgacggacctggtcggctatgtcaaggacatgatcgccaagggtgcgacgcaggtctacgtgttcaacctgcccgacagcagcctgacgccggacggcgtggcaagcggcacgaccggccaggcgctgctgcacgcgctggtgggcacgttcaacacgacgctgcaaagcgggctggccggcacctcggcgcgcatcatcgacttcaacgcacaactgaccgcggcgatccagaatggcgcctcgttcggcttcgccaacaccagcgcccgggcctgcgacgccaccaagatcaatgccctggtgccgagcgccggcggcagctcgctgttctgctcggccaacacgctggtggcttccggtgcggaccagagctacctgttcgccgacggcgtgcacccgaccacggccggccatcgcctgatcgccagcaacgtgctggcgcgcctgctggcggataacgtcgcgcactga

SEQ ID NO: 56 - DNA, 786 bp, Streptomyces coelicolor
gtgatcgggtcgtacgtggcggtgggggacagcttcaccgagggcgtcggcgaccccggccccgacggggcgttcgtcggctgggccgaccggctcgccgtactgctcgcggaccggcgccccgagggcgacttcacgtacacgaacctcgccgtgcgcggcaggctcctcgaccagatcgtggcggaacaggtcccgcgggtcgtcggactcgcgcccgacctcgtctcgttcgcggcgggcggcaacgacatcatccggcccggcaccgatcccgacgaggtcgccgagcggttcgagctggcggtggccgcgctgaccgccgcggccggaaccgtcctggtgaccaccgggttcgacacccggggggtgcccgtcctcaagcacctgcgcggcaagatcgccacgtacaacgggcacgtccgcgccatcgccgaccgctacggctgcccggtgctcgacctgtggtcgctgcggagcgtccaggaccgcagggcgtgggacgccgaccggctgcacctgtcgccggaggggcacacccgggtggcgctgcgcgcggggcaggccctgggcctgcgcgtcccggccgaccctgaccagccctggccgcccctgccgccgcgcggcacgctcgacgtccggcgcgacgacgtgcactgggcgcgcgagtacctggtgccgtggatcgggcgccggctgcggggcgagtcgtcgggcgaccacgtgacggccaaggggacgctgtcgccggacgccatcaagacgcggatcgccgcggtggcctga

SEQ ID NO: 57 - DNA, 783 bp, Streptomyces coelicolor
atgcagacgaaccccgcgtacaccagtctcgtcgccgtcggcgactccttcaccgagggcatgtcggacctgctgcccgacggctcctaccgtggctgggccgacctcctcgccacccggatggcggcccgctcccccggcttccggtacgccaacctggcggtgcgcgggaagctgatcggacagatcgtcgacgagcaggtggacgtggccgccgccatgggagccgacgtgatcacgctggtcggcgggctcaacgacacgctgcggcccaagtgcgacatggcccgggtgcgggacctgctgacccaggccgtggaacggctcgccccgcactgcgagcagctggtgctgatgcgcagtcccggtcgccagggtccggtgctggagcgcttccggccccgcatggaggccctgttcgccgtgatcgacgacctggccgggcggcacggcgccgtggtcgtcgacctgtacggggcccagtcgctggccgaccctcggatgtgggacgtggaccggctgcacctgaccgccgagggccaccgccgggtcgcggaggcggtgtggcagtcgctcggccacgagcccgaggaccccgagtggcacgcgccgatcccggcgacgccgccgccggggtgggtgacgcgcaggaccgcggacgtccggttcgcccggcagcacctgctgccctggataggccgcaggctgaccgggcgctcgtccggggacggcctgccggccaagcgcccggacctgctgccctacgaggaccccgcacggtga

SEQ ID NO: 58 - DNA, 1365 bp, Streptomyces coelicolor
atgacccggggtcgtgacgggggtgcgggggcgccccccaccaagcaccgtgccctgctcgcggcgatcgtcaccctgatagtggcgatctccgcggccatatacgccggagcgtccgcggacgacggcagcagggaccacgcgctgcaggccggaggccgtctcccacgaggagacgccgcccccgcgtccaccggtgcctgggtgggcgcctgggccaccgcaccggccgcggccgagccgggcaccgagacgaccggcctggcgggccgctccgtgcgcaacgtcgtgcacacctcggtcggcggcaccggcgcgcggatcaccctctcgaacctgtacgggcagtcgccgctgaccgtcacacacgcctcgatcgccctggccgccgggcccgacaccgccgccgcgatcgccgacaccatgcgccggctcaccttcggcggcagcgcccgggtgatcatcccggcgggcggccaggtgatgagcgacaccgcccgcctcgccatcccctacggggcgaacgtcctggtcaccacgtactcccccatcccgtccgggccggtgacctaccatccgcaggcccggcagaccagctacctggccgacggcgaccgcacggcggacgtcaccgccgtcgcgtacaccacccccacgccctactggcgctacctgaccgccctcgacgtgctgagccacgaggccgacggcacggtcgtggcgttcggcgactccatcaccgacggcgcccgctcgcagagcgacgccaaccaccgctggaccgacgtcctcgccgcacgcctgcacgaggcggcgggcgacggccgggacacgccccgctacagcgtcgtcaacgagggcatcagcggcaaccggctcctgaccagcaggccggggcggccggccgacaacccgagcggactgagccggttccagcgggacgtgctggaacgcaccaacgtcaaggccgtcgtcgtcgtcctcggcgtcaacgacgtcctgaacagcccggaactcgccgaccgcgacgccatcctgaccggcctgcgcaccctcgtcgaccgggcgcacgcccggggactgcgggtcgtcggcgccacgatcacgccgttcggcggctacggcggctacaccgaggcccgcgagacgatgcggcaggaggtcaacgaggagatccgctccggccgggtcttcgacacggtcgtcgacttcgacaaggccctgcgcgacccgtacgacccgcgccggatgcgctccgactacgacagcggcgaccacctgcaccccggcgacaaggggtacgcgcgcatgggcgcggtcatcgacctggccgcgctgaagggcgcggcgccggtcaaggcgtag

SEQ ID NO: 59 - DNA, 1023 bp, Streptomyces coelicolor
atgacgagcatgtcgagggcgagggtggcgcggcggatcgcggccggcgcggcgtacggcggcggcggcatcggcctggcgggagcggcggcggtcggtctggtggtggccgaggtgcagctggccagacgcagggtgggggtgggcacgccgacccgggtgccgaacgcgcagggactgtacggcggcaccctgcccacggccggcgacccgccgctgcggctgatgatgctgggcgactccacggccgccgggcagggcgtgcaccgggccgggcagacgccgggcgcgctgctggcgtccgggctcgcggcggtggcggagcggccggtgcggctggggtcggtcgcccagccgggggcgtgctcggacgacctggaccggcaggtggcgctggtgctcgccgagccggaccgggtgcccgacatctgcgtgatcatggtcggcgccaacgacgtcacccaccggatgccggcgacccgctcggtgcggcacctgtcctcggcggtacggcggctgcgcacggccggtgcggaggtggtggtcggcacctgtccggacctgggcacgatcgagcgggtgcggcagccgctgcgctggctggcccggcgggcctcacggcagctcgcggcggcacagaccatcggcgccgtcgagcagggcgggcgcacggtgtcgctgggcgacctgctgggtccggagttcgcgcagaacccgcgggagctcttcggccccgacaactaccacccctccgccgaggggtacgccacggccgcgatggcggtactgccctcggtgtgcgccgcgctcggcctgtggccggccgacgaggagcacccggacgcgctgcgccgcgagggcttcctgccggtggcgcgcgcggcggcggaggcggcgtccgaggcgggtacggaggtcgccgccgccatgcctacggggcctcgggggccctgggcgctgctgaagcgccggagacggcgtcgggtgtcggaggcggaaccgtccagcccgtccggcgtttga

SEQ ID NO: 60 - DNA, 918 bp, Streptomyces coelicolor
atgggtcgagggacggaccagcggacgcggtacggccgtcgccgggcgcgtgtcgcgctcgccgccctgaccgccgccgtcctgggcgtgggcgtggcgggctgcgactccgtgggcggcgactcacccgctccttccggcagcccgtcgaagcggacgaggacggcgcccgcctgggacaccagcccggcgtccgtcgccgccgtgggcgactccatcacgcgcggcttcgacgcctgtgcggtgctgtcggactgcccggaggtgtcgtgggcgaccggcagcagcgcgaaggtcgactcgctggccgtacggctgctggggaaggcggacgcggccgagcacagctggaactacgcggtcaccggggcccggatggcggacctgaccgctcaggtgacgcgggcggcgcagcgcgagccggagctggtggcggtgatggccggggcgaacgacgcgtgccggtccacgacctcggcgatgacgccggtggcggacttccgggcgcagttcgaggaggcgatggccaccctgcgcaagaagctccccaaggcgcaggtgtacgtgtcgagcatcccggacctcaagcggctctggtcccagggccgcaccaacccgctgggcaagcaggtgtggaagctcggcctgtgcccgtcgatgctgggcgacgcggactccctggactcggcggcgaccctgcggcgcaacacggtgcgcgaccgggtggcggactacaacgaggtgctgcgggaggtctgcgcgaaggaccggcggtgccgcagcgacgacggcgcggtgcacgagttccggttcggcacggaccagttgagccactgggactggttccacccgagtgtggacggccaggcccggctggcggagatcgcctaccgcgcggtcaccgcgaagaatccctga

SEQ ID NO: 61 - DNA, 1068 bp, Streptomyces rimosus
ttcatcacaacgatgtcacaacaccggccatccgggtcatccctgatcgtgggaatgggtgacaagccttcccgtgacgaaagggtcctgctacatcagaaatgacagaaatcctgctcagggaggttccatgagactgtcccgacgcgcggccacggcgtccgcgctcctcctcaccccggcgctcgcgctcttcggcgcgagcgccgccgtgtccgcgccgcgaatccaggccaccgactacgtggccctcggcgactcctactcctcgggggtcggcgcgggcagctacgacagcagcagtggctcctgtaagcgcagcaccaagtcctacccggccctgtgggccgcctcgcacaccggtacgcggttcaacttcaccgcctgttcgggcgcccgcacaggagacgtgctggccaagcagctgaccccggtcaactccggcaccgacctggtcagcattaccatcggcggcaacgacgcgggcttcgccgacaccatgaccacctgcaacctccagggcgagagcgcgtgcctggcgcggatcgccaaggcgcgcgcctacatccagcagacgctgcccgcccagctggaccaggtctacgacgccatcgacagccgggcccccgcagcccaggtcgtcgtcctgggctacccgcgcttctacaagctgggcggcagctgcgccgtcggtctctcggagaagtcccgcgcggccatcaacgccgccgccgacgacatcaacgccgtcaccgccaagcgcgccgccgaccacggcttcgccttcggggacgtcaacacgaccttcgccgggcacgagctgtgctccggcgccccctggctgcacagcgtcacccttcccgtggagaactcctaccaccccacggccaacggacagtccaagggctacctgcccgtcctgaactccgccacctgatctcgcggctactccgcccctgacgaagtcccgcccccgggcggggcttcgccgtaggtgcgcgtaccgccgtcgcccgtcgcgccggtggccccgccgtacgtgccgccgcccccggacgcggtcggttc

SEQ ID NO: 62 - DNA, 1008 bp, Aeromonas hydrophila
atgaaaaaatggtttgtgtgtttattgggattggtcgcgctgacagttcaggcagccgacagtcgccccgccttttcccggatcgtgatgttcggcgacagcctctccgataccggcaaaatgtacagcaagatgcgcggttacctcccctccagcccgccctactatgagggccgtttctccaacggacccgtctggctggagcagctgaccaaacagttcccgggtctgaccatcgccaacgaagcggaaggcggtgccactgccgtggcttacaacaagatctcctggaatcccaagtatcaggtcatcaacaacctggactacgaggtcacccagttcttgcagaaagacagcttcaagccggacgatctggtgatcctctgggtcggtgccaatgactatctggcctatggctggaacacggagcaggatgccaagcgggttcgcgatgccatcagcgatgcggccaaccgcatggtactgaacggtgccaagcagatactgctgttcaacctgccggatctgggccagaacccgtcagctcgcagtcagaaggtggtcgaggcggtcagccatgtctccgcctatcacaaccagctgctgctgaacctggcacgccagctggcccccaccggcatggtaaagctgttcgagatcgacaagcaatttgccgagatgctgcgtgatccgcagaacttcggcctgagcgacgtcgagaacccctgctacgacggcggctatgtgtggaagccgtttgccacccgcagcgtcagcaccgaccgccagctctccgccttcagtccgcaggaacgcctcgccatcgccggcaacccgctgctggcacaggccgttgccagtcctatggcccgccgcagcgccagccccctcaactgtgagggcaagatgttctgggatcaggtacacccgaccactgtcgtgcacgcagccctgagcgagcgcgccgccaccttcatcgcgaaccagtacgagttcctcgcccactga

SEQ ID NO: 63 - DNA, 1011 bp, Aeromonas salmonicida subsp. Salmonicida
atgaaaaaatggtttgtttgtttattggggttgatcgcgctgacagttcaggcagccgacactcgccccgccttctcccggatcgtgatgttcggcgacagcctctccgataccggcaaaatgtacagcaagatgcgcggttacctcccctccagcccgccctactatgagggccgtttctccaacggacccgtctggctggagcagctgaccaagcagttcccgggtctgaccatcgccaacgaagcggaaggcggtgccactgccgtggcttacaacaagatctcctggaatcccaagtatcaggtcatcaacaacctggactacgaggtcacccagttcttgcagaaagacagcttcaagccggacgatctggtgatcctctgggtcggtgccaatgactatctggcatatggctggaatacggagcaggatgccaagcgagttcgcgatgccatcagcgatgcggccaaccgcatggtactgaacggtgccaagcagatactgctgttcaacctgccggatctgggccagaacccgtcagcccgcagtcagaaggtggtcgaggcggtcagccatgtctccgcctatcacaacaagctgctgctgaacctggcacgccagctggcccccaccggcatggtaaagctgttcgagatcgacaagcaatttgccgagatgctgcgtgatccgcagaacttcggcctgagcgacgtcgagaacccctgctacgacggcggctatgtgtggaagccgtttgccacccgcagcgtcagcaccgaccgccagctctccgccttcagtccgcaggaacgcctcgccatcgccggcaacccgctgctggcacaggccgttgccagtcctatggcccgccgcagcgccagccccctcaactgtgagggcaagatgttctgggatcaggtacacccgaccactgtcgtgcacgcagccctgagcgagcgcgccgccaccttcatcgagacccagtacgagttcctcgcccacggatga

SEQ ID NO: 64 - DNA, 51 bp, Artificial Sequence
  alpha-amylase terminator sequence
cgggacttaccgaaagaaaccatcaatgatggtttcttttttgttcataaa

SEQ ID NO: 65 - DNA, 59 bp, Artificial Sequence
  alkaline protease terminator sequence
caagactaaagaccgttcgcccgtttttgcaataagcgggcgaatcttacataaaaata

SEQ ID NO: 66 - DNA, 61 bp, Artificial Sequence
  glutamic-acid specific terminator sequence
acggccgttagatgtgacagcccgttccaaaaggaagcgggctgtcttcgtgtattattgt

SEQ ID NO: 67 - DNA, 54 bp, Artificial Sequence
  levanase terminator sequence
tcttttaaaggaaaggctggaatgcccggcattccagccacatgatcatcgttt

SEQ ID NO: 68 - protein, 280 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFRSASPLNCEGKMFWDQVHPTTVVHAALSERAATFIETQYEFLAHG

SEQ ID NO: 69 - protein, 4 aa, Artificial Sequence
  Amino acid sequence motif
  MISC_FEATURE (4)..(4): Xaa may be Leu, Ala, Val, Ile, Phe, Tyr, His, Gln, Thr, Asn, Met, or Ser.
GDSX

SEQ ID NO: 70 - protein, 5 aa, Artificial Sequence
  Peptide motif
GGNDA

SEQ ID NO: 71 - protein, 5 aa, Artificial Sequence
  Peptide motif
GGNDL

SEQ ID NO: 72 - protein, 5 aa, Artificial Sequence
  Peptide motif
  misc_feature (5)..(5): Xaa can be any naturally occurring amino acid
GGNDX

SEQ ID NO: 73 - protein, 8 aa, Artificial Sequence
  Consensus sequence
MRRSRFLA

SEQ ID NO: 74 - protein, 8 aa, Artificial Sequence
  Consensus sequence
ALILLTLA

SEQ ID NO: 75 - protein, 5 aa, Artificial Sequence
  Consensus sequence
ARAAP

SEQ ID NO: 76 - protein, 11 aa, Artificial Sequence
  Consensus sequence
YVALGDSYSSG

SEQ ID NO: 77 - protein, 5 aa, Artificial Sequence
  Consensus sequence
GAGSY

SEQ ID NO: 78 - protein, 4 aa, Artificial Sequence
  Consensus sequence
SSGD

SEQ ID NO: 79 - protein, 15 aa, Artificial Sequence
  Consensus sequence
RSTKAYPALWAAAHA

SEQ ID NO: 80 - protein, 5 aa, Artificial Sequence
  Consensus sequence
SSFSF

SEQ ID NO: 81 - protein, 12 aa, Artificial Sequence
  Consensus sequence
ACSGARTYDVLA

SEQ ID NO: 82 - protein, 15 aa, Artificial Sequence
  Consensus sequence
LVSITIGGNDAGFAD

SEQ ID NO: 83 - protein, 6 aa, Artificial Sequence
  Consensus sequence
MTTCVL

SEQ ID NO: 84 - protein, 6 aa, Artificial Sequence
  Consensus sequence
SDSACL

SEQ ID NO: 85 - protein, 4 aa, Artificial Sequence
  Consensus sequence
TLPA

SEQ ID NO: 86 - protein, 9 aa, Artificial Sequence
  Consensus sequence
RLDSVYSAI

SEQ ID NO: 87 - protein, 4 aa, Artificial Sequence
  Consensus sequence
TRAP

SEQ ID NO: 88 - protein, 12 aa, Artificial Sequence
  Consensus sequence
ARVVVLGYPRIY

SEQ ID NO: 89 - protein, 4 aa, Artificial Sequence
  Consensus sequence
LGLS

SEQ ID NO: 90 - protein, 11 aa, Artificial Sequence
  Consensus sequence
TKRAAINDAAD

SEQ ID NO: 91 - protein, 12 aa, Artificial Sequence
  Consensus sequence
LNSVIAKRAADH

SEQ ID NO: 92 - protein, 7 aa, Artificial Sequence
  Consensus sequence
GFTFGDV

SEQ ID NO: 93 - protein, 7 aa, Artificial Sequence
  Consensus sequence
GHELCSA

SEQ ID NO: 94 - protein, 9 aa, Artificial Sequence
  Consensus sequence
PWLHSLTLP

SEQ ID NO: 95 - protein, 6 aa, Artificial Sequence
  Consensus sequence
SYHPTA

SEQ ID NO: 96 - protein, 13 aa, Artificial Sequence
  Consensus sequence
GHAAGYLPVLNSI

SEQ ID NO: 97 - DNA, 1225 bp, Artificial Sequence
  XhoI insert sequence containing the LAT-KLM3′ precursor gene
gcttttcttttggaagaaaatatagggaaaatggtacttgttaaaaattcggaatatttatacaatatcatatgtttcacattgaaaggggaggagaatcatgaaacaacaaaaacggctttacgcccgattgctgacgctgttatttgcgctcatcttcttgctgcctcattctgcagcttcagcagcagatacaagaccggcgtttagccggatcgtcatgtttggagatagcctgagcgatacgggcaaaatgtatagcaaaatgagaggctatcttccgtcaagcccgccgtattatgaaggccgctttagcaatggaccggtctggctggaacaactgacgaaacaatttccgggactgacgatcgctaatgaagcagaaggaggagcaacagcggtcgcctataacaaaatcagctgggacccgaaatatcaggtcatcaacaacctggactatgaagtcacacagtttcttcagaaagacagctttaaaccggatgatctggtcatcctttgggtcggcgccaatgattatctggcgtatggctggaacacagaacaagatgccaaaagagtcagagatgccatcagcgatgccgctaatagaatggtcctgaacggcgccaaacaaatcctgctgtttaacctgccggatctgggacaaaatccgagcgccagaagccaaaaagtcgtcgaagcagtcagccatgtcagcgcctatcataacaaactgctgctgaacctggcaagacaattggcaccgacgggaatggttaaattgtttgaaattgacaaacagtttgccgaaatgctgagagatccgcaaaattttggcctgagcgatgtcgaaaacccgtgctatgatggcggatatgtctggaaaccgtttgccacaagaagcgtcagcacggatagacaactgtcagcgtttagcccgcaagaaagactggcaatcgccggaaatccgcttttggcacaagcagttgcttcaccgatggcaagaagatcagcaagcccgctgaattgcgaaggcaaaatgttttgggatcaggtccatccgacaacagttgtccatgctgccctttcagaaagagcggcgacgtttatcgaaacacagtatgaatttctggcccatggctgagttaacagaggacggatttcctgaaggaaatccgtttttttattttaagcttggagacaaggtaaaggataaaacctcgag

SEQ ID NO: 98 - protein, 347 aa, Artificial Sequence
  XhoI insert sequence containing the LAT-KLM3′ precursor gene
MKQQKRLYARLLTLLFALIFLLPHSAASAADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIETQYEFLAHG

SEQ ID NO: 99-118 - skipped (20 entries)

SEQ ID NO: 119 - DNA, 56 bp, Artificial Sequence
  subtilisin E terminator sequence
gctgacaaataaaaagaagcaggtatggaggaacctgcttctttttactattattg

SEQ ID NO: 120 - DNA, 954 bp, Aeromonas salmonicida
gacactcgccccgccttctcccggatcgtgatgttcggcgacagcctctccgataccggcaaaatgtacagcaagatgcgcggttacctcccctccagcccgccctactatgagggccgtttctccaacggacccgtctggctggagcagctgaccaagcagttcccgggtctgaccatcgccaacgaagcggaaggcggtgccactgccgtggcttacaacaagatctcctgggaccccaagtatcaggtcatcaacaacctggactacgaggtcacccagttcttgcagaaagacagcttcaagccggacgatctggtgatcctctgggtcggtgccaatgactatctggcatatggctggaatacggagcaggatgccaagcgagttcgcgatgccatcagcgatgcggccaaccgcatggtactgaacggtgccaagcagatactgctgttcaacctgccggatctgggccagaacccgtcagcccgcagtcagaaggtggtcgaggcggtcagccatgtctccgcctatcacaacaagctgctgctgaacctggcacgccagctggcccccaccggcatggtaaagctgttcgagatcgacaagcaatttgccgagatgctgcgtgatccgcagaacttcggcctgagcgacgtcgagaacccctgctacgacggcggctatgtgtggaagccgtttgccacccgcagcgtcagcaccgaccgccagctctccgccttcagtccgcaggaacgcctcgccatcgccggcaacccgctgctggcacaggccgttgccagtcctatggcccgccgcagcgccagccccctcaactgtgagggcaagatgttctgggatcaggtacacccgaccactgtcgtgcacgcagccctgagcgagcgcgccgccaccttcatcgagacccagtacgagttcctcgcccacggatga

SEQ ID NO: 121 - protein, 279 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFSASPLNCEGKMFWDQVHPTTVVHAALSERAATFIETQYEFLAHG

SEQ ID NO: 122 - protein, 278 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFASPLNCEGKMFWDQVHPTTVVHAALSERAATFIETQYEFLAHG

SEQ ID NO: 123 - protein, 277 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFSPLNCEGKMFWDQVHPTTVVHAALSERAATFIETQYEFLAHG

SEQ ID NO: 124 - protein, 122 aa, Streptomyces violaceoruber
APADKPQVLASFTQTSASSQNAWLAANRNQSAWAAYEFDWSTDLCTQAPDNPFGFPFNTACARHDFGYRNYKAAGSFDANKSRIDSAFYEDMKRVCTGYTGEKNTACNSTAWTYYQAVKIFG

(19) United States
(12) Patent Application Publication
(10) Pub. No.: US 2017/0121741 A1
(43) Pub. Date: May 4, 2017

(54) METHOD FOR INCREASING CRUDE PALM OIL YIELDS

(71) Applicant: DUPONT NUTRITION BIOSCIENCES APS, Copenhagen K (DK)

(72) Inventors: Jorn Borch Soe, Tilst (DK); Lai See Lee, Singapore (SG)

(21) Appl. No.: 15/115,046

(22) PCT Filed: Mar. 31, 2015
(86) PCT No.: PCT/EP2015/057002

§ 371 Date: Jul. 28, 2016

(30) Foreign Application Priority Data
Apr. 1, 2014 (MY) PI 2014700786
Dec. 19, 2014 (MY) PI 2014703899

(51) Int. Cl. C12P 7/64; C11B 1/04; C11B 1/06; C11B 1/02

(52) U.S. Cl. CPC C12P 7/64; C11B 1/025; C11B 1/04; C11B 1/06

(57) ABSTRACT

A method for improving crude palm oil yields or separating the crude palm oil from the sludge or a combination thereof in palm fruit processing comprising: admixing a palm fruit or a portion thereof or a palm fruit extract and an enzyme, which enzyme degrades a phospholipid present in said palm fruit or portion thereof or palm fruit extract; and incubating the admixture at about 45° C. to about 95° C. for about 15 minutes to about 6 hours. Also included are uses of an enzyme which degrades a phospholipid.

### FIELD OF THE INVENTION

The present invention relates to a method for the treatment of palm fruit or a portion thereof or palm fruit extract (or pressed palm fruit extract also known in the industry as “pressed liquid” or “pressed oil”) with an enzyme that degrades a phospholipid present in the palm fruit extract. The method results in de-emulsification of the palm fruit extract (e.g. pressed palm fruit extract) and significantly improved separation of oil from pressed palm liquid, thus reducing the amount of oil in the waste stream.

### BACKGROUND

Palm oil obtained from oil palm (Elaeis guineensis) is commercially important edible oil. Palm oil has been a prominent fat and oil resource for the food industry due to several advantageous properties, such as high productivity, low price, high thermal and oxidative stability and plasticity at room temperature. In addition, compared with other vegetable oils, palm oil is a rich source of the anti-oxidant vitamin E.

In 2012, the world production of palm oil was 50 mill ton/year, which in quantity makes it the most important vegetable oil produced. It is estimated that 4-8% of palm oil is lost during processing. This loss can be split up as follows: 0.8-1% is lost in the palm mesocarp fibre, 1.5-2.7% is lost from the empty fruit bunch (EFB); and more than about 1% is lost in oil mill effluent (Ho et al JOACS, Vol. 69, No. 3 Mar. 1992). The percentage calculation is based on fresh fruit bunches.

The processing of palm oil is complicated and extensive. FIG. 1 shows flow diagram for palm oil processing. Production of crude palm oil is conducted by a series of unit operations starting with a sterilization of the fresh fruit bunch (FFB).

After sterilization, the fruits are stripped from the bunch and digested. During the digestion, the palm fruit is disintegrated and oil released from the fiber. After digestion, the crude oil is separated from the fiber by pressing the digested fruits.

The pressed palm oil thus obtained is a mixture of oil and water, and the oil phase is isolated by separation in a clarifying tank.

Crude palm oil that is discharged from the presses is highly viscous. Thus separation of the oil from the solid and water is difficult without the addition of dilution water. Hot water is therefore added to the pressed liquid to dilute it prior to or during clarification. This typically occurs at temperatures of 80-90° C. The dilution provides a barrier causing the heavy solid to settle to the bottom of the clarification tank while the lighter oil droplets rise through the sludge phase to the top when heat is applied. In practice it has been found that dilution with water such that 38% to 40% of the mixture is crude oil is best for good separation in the clarification tank. Any remaining available oil post clarification is removed by centrifugation. The centrifuge sludge is a viscous liquid containing water, about 0.5-1.5% oil and 5-10% non-oil solids. For each ton of oil produced, 1-1.5 ton of centrifuge sludge is produced. The centrifuge sludge thus presents a substantial loss of oil.

During these initial processing steps 90-92% of the theoretical palm oil amount is obtained, but there is a significant oil loss to the fiber (e.g. press cake) during processing as well as oil loss during downstream processing.

It is known to use enzymes in the processing of vegetable oils. Enzymes such as phospholipases or lipid acyltransferases have been used to increase the oil yield in enzymatic and/or water degumming of oil with a high content of phospholipids (see U.S. Pat. No. 6,001,640, WO2006/008508, WO2009/081094 for example).

In these reactions the enzyme is added to water-degummed edible oils, crude edible oils or semi-crude edible oils comprising relatively high amounts of a non-hydratable phosphorus ranging from about 50 to about 3000 ppm. In the flow diagram shown herein in FIG. 1 the type of oil which would have been used in such processes is designated “crude palm oil”. In any event degumming of palm oil is often not essential as the level of non-hydratable phosphorus can be naturally low in this product, especially in comparison to other vegetable oils. For example the phosphorus content of palm oil is about 15-30 ppm which is very low when compared to e.g. corn oil (250-800 ppm), cottonseed (400-1000 ppm), rapeseed (200-1400 ppm), soya (400-1200 ppm) or sunflower (200-500 ppm).

Traditionally, palm oil is produced by pressing the oil out of the palm mesocarp without use of organic solvents.

### SUMMARY OF THE INVENTION

According to a first aspect the present invention provides a method for improving crude palm oil yields or enhancing separation of the crude palm oil or a combination thereof in palm fruit processing comprising:
a. admixing a palm fruit or a portion thereof or a palm fruit extract and an enzyme, which enzyme degrades a phospholipid present in said palm fruit or a portion thereof or palm fruit extract, and
b. incubating the admixture at about 45° C. to about 95° C. for about 15 minutes to about 6 hours.

According to a second aspect the present invention provides the use of an enzyme, which enzyme degrades a phospholipid present in palm fruit or a portion thereof or a palm fruit extract for improving crude palm oil yields in palm fruit processing or improving separation of the crude palm oil (e.g. in the clarifier) or a combination thereof.

### BRIEF DESCRIPTION OF THE DRAWINGS

Embodiments of the invention will now be described, by way of example only, with reference to accompanying drawings, in which:

FIG. 1 shows a flow diagram for Palm Oil processing.

FIG. 2 shows the cleavage sites on a phospholipid for different phospholipases.

FIG. 3 shows the amino acid sequence of a mutant Aeromonas salmonicida mature lipid acyttransferase (GCAT) with a mutation of Asn80Asp (notably, amino acid 80 is in the mature sequence) (SEQ ID No. 16);

FIG. 4 shows an amino acid sequence (SEQ ID No. 1) of a lipid acyl transferase from Aeromonas hydrophila (ATCC #7965);

FIG. 5 shows a pfam00657 consensus sequence from database version 6 (SEQ ID No. 2);

FIG. 6 shows an amino acid sequence (SEQ ID No. 3) obtained from the organism Aeromonas hydrophila (P10480; GI:121051);

FIG. 7 shows an amino acid sequence (SEQ ID No. 4) obtained from the organism Aeromonas salmonicida (AAG098404; GI:9964017);

FIG. 8 shows an amino acid sequence (SEQ ID No. 5) obtained from the organism Streptomyces coelicolor A3(2) (Genbank accession number NP_631558);

FIG. 9 shows an amino acid sequence (SEQ ID No. 6) obtained from the organism Streptomyces coelicolor A3(2) (Genbank accession number CAC42140);

FIG. 10 shows an amino acid sequence (SEQ ID No. 7) obtained from the organism Saccharomyces cerevisiae (Genbank accession number P41734);

FIG. 11 shows an amino acid sequence (SEQ ID No. 8) obtained from the organism Ralstonia (Genbank accession number AL646052);

FIG. 12 shows SEQ ID No. 9. Scoe1 NCBI protein accession code CAB39707.1 GI:4539178 conserved hypothetical protein [Streptomyces coelicolor A3(2)];

FIG. 13 shows an amino acid shown as SEQ ID No. 10. Scoe2 NCBI protein accession code CAC01477.1 GI:9716139 conserved hypothetical protein [Streptomyces coelicolor A3(2)];

FIG. 14 shows an amino acid sequence (SEQ ID No. 11) Scoe3 NCBI protein accession code CAB88833.1 GI:7635996 putative secreted protein [Streptomyces coelicolor A3(2)];

FIG. 15 shows an amino acid sequence (SEQ ID No. 12) Scoe4 NCBI protein accession code CAB89450.1 GI:7672261 putative secreted protein [Streptomyces coelicolor A3(2)];

FIG. 16 shows an amino acid sequence (SEQ ID No. 13) Scoe5 NCBI protein accession code CAB62724.1 GI:6562793 putative lipoprotein [Streptomyces coelicolor A3(2)];

FIG. 17 shows an amino acid sequence (SEQ ID No. 14) Srim1 NCBI protein accession code AAK84028.1 GI:15082088 GDSL-lipase [Streptomyces rimosus];

FIG. 18 shows an amino acid sequence (SEQ ID No. 15) of a lipid acyltransferase from Aeromonas salmonicida subsp. Salmonicida (ATCC#14174);

FIG. 19 shows SEQ ID No. 19. Scoe1 NCBI protein accession code CAB39707.1 GI:4539178 conserved hypothetical protein [Streptomyces coelicolor A3(2)];

FIG. 20 shows an amino acid sequence (SEQ ID No. 25) of the fusion construct used for mutagenesis of the Aeromonas hydrophila lipid acyltransferase gene. The underlined amino acids is a xylanase signal peptide;

FIG. 21 shows a polypeptide sequence of a lipid acyttransferase enzyme from Streptomyces (SEQ ID No. 26);

FIG. 22 shows a polypeptide sequence of a lipid acyltransferase enzyme from Thermobifida (SEQ ID No. 27);

FIG. 23 shows a polypeptide sequence of a lipid acyltransferase enzyme from Thermobifida (SEQ ID No. 28);

FIG. 24 shows a polypeptide of a lipid acyltransferase enzyme from Corynebacterium efficiens GDSx 300 amino acid (SEQ ID No. 29);

FIG. 25 shows a polypeptide of a lipid acyitransferase enzyme from Novosphingobium aromaticivorans GDSx 284 amino acid (SEQ ID No. 30);

FIG. 26 shows a polypeptide of a lipid acyltransferase enzyme from Streptomyces coelicolor GDSx 269 aa (SEQ ID No. 31);

FIG. 27 shows a polypeptide of a lipid acyltransferase enzyme from Streptomyces avermitilis\GDSx 269 amino acid (SEQ ID No. 32);

FIG. 28 shows a polypeptide of a lipid acyltransferase enzyme from Streptomyces (SEQ ID No. 33);

FIG. 29 shows an amino acid sequence (SEQ ID No. 34) obtained from the organism Aeromonas hydrophila (P10480; GI:121051) (notably, this is the mature sequence);

FIG. 30 shows the amino acid sequence (SEQ ID No. 35) of Aeromonas salmonicida mature lipid acyltransferase (GCAT) (notably, this is the mature sequence);

FIG. 31 shows a nucleotide sequence (SEQ ID No. 36) from Streptomyces thermosacchari;

FIG. 32 shows an amino acid sequence (SEQ ID No. 37) from Streptomyces thermosacchari;

FIG. 33 shows an amino acid sequence (SEQ ID No. 38) from Thermobifida fusca/GDSx 548 amino acid;

FIG. 34 shows a nucleotide sequence (SEQ ID No. 39) from Thermobifida fusca;

FIG. 35 shows an amino acid sequence (SEQ ID No. 40) from Thermobifida fusca/GDSx;

FIG. 36 shows an amino acid sequence (SEQ ID No. 41) from Corynebacterium efficiens/GDSx 300 amino acid;

FIG. 37 shows a nucleotide sequence (SEQ ID No. 42) from Corynebacterium efficiens;

FIG. 38 shows an amino acid sequence (SEQ ID No. 43) from S. coelicolor/GDSx 268 amino acid;

FIG. 39 shows a nucleotide sequence (SEQ ID No. 44) from S. coelicolor;

FIG. 40 shows an amino acid sequence (SEQ ID No. 45) from S. avermitilis;

FIG. 41 shows a nucleotide sequence (SEQ ID No. 46) from S. avermitilis;

FIG. 42 shows an amino acid sequence (SEQ ID No. 47) from Thermobifida fusca/GDSx;

FIG. 43 shows a nucleotide sequence (SEQ ID No. 48) from Thermobifida fusca/GDSx;

FIG. 44 shows an alignment of the L131 and homologues from S. avermitilis and T. fusca illustrates that the conservation of the GDSx motif (GDSY in L131 and S. avermitilis and T. fusca), the GANDY box, which is either GGNDA or GGNDL, and the HPT block (considered to be the conserved catalytic histidine). These three conserved blocks are highlighted;

FIG. 45 shows SEQ ID No 17 which is the amino acid sequence of a lipid acyttransferase from Candida parapsilosis;

FIG. 46 shows SEQ ID No 18 which is the amino acid sequence of a lipid acyttransferase from Candida parapsilosis;

FIG. 47 shows the sequence of the Xhol insert containing the LAT-KLM3′ precursor gene, the −35 and -10 boxes are underlined;

FIG. 48 shows a nucleotide sequence from Aeromonas salmonicida (SEQ ID No. 49) including the signal sequence (preLAT—positions 1 to 87);

FIG. 49 shows a nucleotide sequence (SEQ ID No. 50) encoding a lipid acyl transferase according to the present invention obtained from the organism Aeromonas hydrophila;

FIG. 50 shows a nucleotide sequence (SEQ ID No. 51) encoding a lipid acyl transferase according to the present invention obtained from the organism Aeromonas salmonicida;

FIG. 51 shows a nucleotide sequence (SEQ ID No. 52) encoding a lipid acyl transferase according to the present invention obtained from the organism Streptomyces coelicolor A3(2) (Genbank accession number NC_003888.1:8327480 . . . 8328367);

FIG. 52 shows a nucleotide sequence (SEQ ID No. 53) encoding a lipid acyl transferase according to the present invention obtained from the organism Streptomyces coelicolor A3(2) (Genbank accession number AL939131.1:265480 . . . 266367);

FIG. 53 shows a nucleotide sequence (SEQ ID No. 54) encoding a lipid acyl transferase according to the present invention obtained from the organism Saccharomyces cerevisiae (Genbank accession number Z75034);

FIG. 54 shows a nucleotide sequence (SEQ ID No. 55) encoding a lipid acyl transferase according to the present invention obtained from the organism Ralstonia;

FIG. 55 shows a nucleotide sequence shown as SEQ ID No. 56 encoding NCBI protein accession code CAB39707.1 GI:4539178 conserved hypothetical protein [Streptomyces coelicolor A3(2)];

FIG. 56 shows a nucleotide sequence shown as SEQ ID No. 57 encoding Scoe2 NCBI protein accession code CAC01477.1 GI:9716139 conserved hypothetical protein [Streptomyces coelicolor A3(2)];

FIG. 57 shows a nucleotide sequence shown as SEQ ID No. 58 encoding Scoe3 NCBI protein accession code CAB88833.1 GI:7635996 putative secreted protein. [Streptomyces coelicolor A3(2)];

FIG. 58 shows a nucleotide sequence shown as SEQ ID No. 59 encoding Scoe4 NCBI protein accession code CAB89450.1 GI:7672261 putative secreted protein. [Streptomyces coelicolor A3(2)];

FIG. 59 shows a nucleotide sequence shown as SEQ ID No. 60, encoding Scoe5 NCBI protein accession code CAB62724.1 GI:6562793 putative lipoprotein [Streptomyces coelicolor A3(2)];

FIG. 60 shows a nucleotide sequence shown as SEQ ID No. 61 encoding Srim1 NCBI protein accession code AAK84028.1 GI:15082088 GDSL-lipase [Streptomyces rimosus];

FIG. 61 shows a nucleotide sequence (SEQ ID No. 62) encoding a lipid acyltransferase from Aeromonas hydrophila (ATCC #7965);

FIG. 62 shows a nucleotide sequence (SEQ ID No 63) encoding a lipid acyltransferase from Aeromonas salmonicida subsp. Salmonicida (ATCC#14174);

FIG. 63 shows the amino acid sequence of a mutant Aeromonas salmonicida mature lipid acyltransferase (GCAT) with a mutation of Asn80Asp (notably, amino acid 80 is in the mature sequence)—shown herein as SEQ ID No. 16—and after undergoing post-translational modification as SEQ ID No. 68—amino acid residues 235 and 236 of SEQ ID No. 68 are not covalently linked following post-translational modification. The two peptides formed are held together by one or more S—S bridges. Amino acid 236 in SEQ ID No. 68 corresponds with the amino acid residue number 274 in SEQ ID No. 16 shown herein;

FIG. 64 shows a nucleotide sequence (SEQ ID NO. 120) which encodes a lipid acyltransferase from A. salmonicida;

FIG. 65 shows the amino acid sequence of a mutant Aeromonas salmonicida mature lipid acyttransferase (GCAT) with a mutation of Asn80Asp (notably, amino acid 80 is in the mature sequence)—shown herein as SEQ ID No. 16—and after undergoing post-translational modification as SEQ ID No. 121—amino acid residues 235 and 236 of SEQ ID No. 121 are not covalently linked following post-translational modification; the two peptides formed are held together by one or more S—S bridges; amino acid 236 in SEQ ID No. 121 corresponds with the amino acid residue number 275 in SEQ ID No. 16 shown herein;

FIG. 66 shows the amino acid sequence of a mutant Aeromonas salmonicida mature lipid acyttransferase (GCAT) with a mutation of Asn80Asp (notably, amino acid 80 is in the mature sequence)—shown herein as SEQ ID No. 16—and after undergoing post-translational modification as SEQ ID No. 122—amino acid residues 235 and 236 of SEQ ID No. 122 are not covalently linked following post-translational modification; the two peptides formed are held together by one or more S—S bridges; amino acid 236 in SEQ ID No. 122 corresponds with the amino acid residue number 276 in SEQ ID No. 16 shown herein; and

FIG. 67 shows the amino acid sequence of a mutant Aeromonas salmonicida mature lipid acyttransferase (GCAT) with a mutation of Asn80Asp (notably, amino acid 80 is in the mature sequence)—shown herein as SEQ ID No. 16—and after undergoing post-translational modification as SEQ ID No. 123—amino acid residues 235 and 236 of SEQ ID No. 123 are not covalently linked following post-translational modification; the two peptides formed are held together by one or more S—S bridges: amino acid 236 in SEQ ID No. 123 corresponds with the amino acid residue number 277 in SEQ ID No. 16 shown herein.

FIG. 68 shows oil separation after enzyme treatment and enzyme inactivation.

FIG. 69 shows oil separation after enzyme treatment and enzyme inactivation (see Table 4).

FIG. 70 shows pressed oil treated with Lecitase Ultra, after heat inactivation.

FIG. 71 shows oil samples after heat inactivation.

FIG. 72 shows oil samples after enzyme treatment and heat inactivation.

FIG. 73 shows pressed oil treated with LysoMax Oil after heat inactivation.

FIG. 74 shows samples (see Table 13) after centrifugation at 48 rcf for 5 minutes at 60° C.

FIG. 75 shows samples (see Table 13) after centrifugation at 3050 rcf for 10 minutes at 60° C.

FIG. 76 shows a sequence (SEQ ID No. 124) which is the sequence of GL541 PLA2.

### DETAILED DESCRIPTION

A seminal finding of the present invention is that treatment of palm fruit extract (e.g. pressed palm fruit extract also known in the industry as “pressed liquid” or “pressed oil”) with an enzyme that degrades a phospholipid in the palm fruit extract results in de-emulsification of the palm fruit extract and significantly improved palm oil yields.

Based on these findings, we provide a method for improving crude palm oil yields or enhancing separation of the crude palm oil (e.g. during clarification) or a combination thereof in palm fruit processing comprising:
a. admixing a palm fruit or a portion thereof or a palm fruit extract and an enzyme, which enzyme degrades a phospholipid present in said palm fruit or a portion thereof or palm fruit extract, and
b. incubating the admixture at about 45° C. to about 95° C. for about 15 minutes to about 6 hours.

Also provided is the use of an enzyme, which enzyme degrades a phospholipid present in palm fruit or a portion thereof or a palm fruit extract for improving crude palm oil yields in palm fruit processing or enhancing separation of the crude palm oil (e.g. during clarification) or a combination thereof.

In another embodiment there is provided a method for processing palm fruit comprising:
a. admixing a palm fruit or a portion thereof or a palm fruit extract and an enzyme, which enzyme produces a lysophospholipid from a phospholipid present in said palm fruit or a portion thereof or palm fruit extract, and
b. incubating the admixture at about 45° C. to about 95° C. for about 15 minutes to about 6 hours.

In one embodiment there is provided the use of an enzyme, which enzyme produces a lysophospholipid from a phospholipid present in palm fruit or a portion thereof or a palm fruit extract for improving crude palm oil yields in palm fruit processing or enhancing separation of the crude palm oil (e.g. during clarification) or a combination thereof.

The enzyme that degrades a phospholipid present in the pressed palm fruit extract as used herein, may be selected from the group consisting of: a lipid acyltransferase, a phospholipase A1, a phospholipase A2, phospholipase B or a phospholipase D.

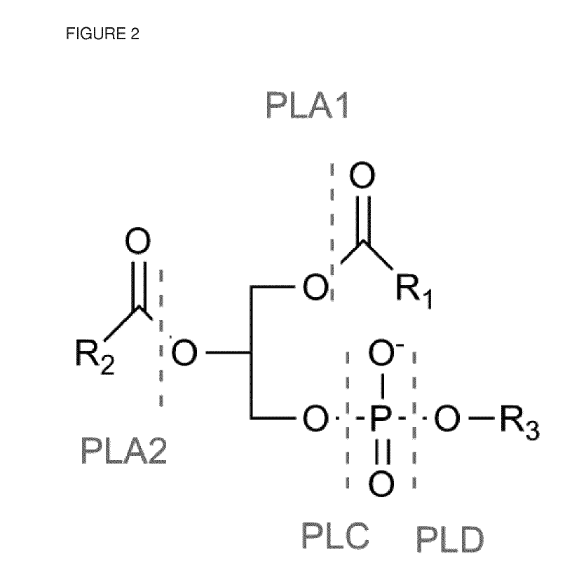

An “enzyme that degrades a phospholipid” as used herein is an enzyme which is able to cleave a bond present in a phospholipid to breakdown the phospholipid molecule into one or more different products, wherein the product(s) does not include phosphocholine or O-Phosphorylethanolamine. Cleavage can occur at the SN-1 position, SN-2 position, after the phosphate group of the phospholipid or a combination thereof. An “enzyme that degrades a phospholipid” is not an enzyme that cleaves a phospholipid before the phosphate. In other words the enzyme that degrades a phospholipid is not a phospholipid C [PLC] (e.g. as shown in FIG. 2).

Without wishing to be bound by theory it is believed that the oil body in the oil-in-water emulsion is covered in fibre and that the degraded phospholipid (e.g. surface acting material) displaces fibre from the oil body. The degraded phospholipid (e.g. surface acting material) is believed to displace macromolecules that are adsorbed at the oil drop surface thereby decreasing the density and rendering them separable upon centrifugation.

The term phospholipid as used herein means any phospholipid, such as a lecithin, e.g. phosphatidylcholine and/or phophatidylethanolamine. The term lecithin as used herein encompasses phosphatidylcholine, phosphatidylethanolamine, phosphatidylinositol and phosphatidic acid (PA).

In one embodiment the enzyme that degrades a phospholipid may be used in combination with an enzyme comprising one or more of the following enzyme activities: cellulase activity, mannanase activity, pectinase activity, xylanase activity, glucuronidase activity, or galactanase.

In one embodiment the enzyme that degrades a phospholipid may be used in combination with an enzyme comprising one or more of the following enzyme activities: cellulase activity and/or mannanase activity.

The cellulase activity according to the present invention may be an endoglucanase (e.g. a 3-glucanase) activity.

The terms “cellulases” or “cellulolytic enzymes” as used herein are understood as comprising endo-glucanase (EC 3.2.1.4) activity.

In one embodiment the cellulase used in accordance with the present invention is an endoglucanase (EC 3.2.1.4), e.g. an endoglucanase that cuts the cellulose chains at random.

The cellulases may comprise a carbohydrate-binding module (CBM) which enhances the binding of the enzyme to a cellulose-containing fiber and increases the efficacy of the catalytic active part of the enzyme. A CBM is defined as contiguous amino acid sequence within a carbohydrate-active enzyme with a discrete fold having carbohydrate-binding activity. For further information of CBMs see the CAZy internet server (Supra) or Tomme et al. (1995) in Enzymatic Degradation of Insoluble Polysaccharides (Saddler and Penner, eds.), Cellulose-binding domains: classification and properties, pp. 142-163, American Chemical Society, Washington. In a preferred embodiment the cellulases or cellulolytic enzymes may be a cellulolytic preparation as defined in U.S. application No. 60/941,251, which is hereby incorporated by reference. In some embodiments the cellulase enzyme is one derived from Trichoderma reesei.

The cellulolytic activity may, in some embodiments, be derived from a fungal source, such as a strain of the genus Trichoderma, such as a strain of Trichoderma reesei; or a strain of the genus Humicola, such as a strain of Humicola insolens.

Endoglucanases (E.C. 3.2.1.4) catalyse endo-hydrolysis of 1,4-beta-D-glycosidic linkages in cellulose, cellulose derivatives (such as carboxy methyl cellulose and hydroxy ethyl cellulose), lichenin, beta-1,4 bonds in mixed beta-1,3 glucans such as cereal beta-D-glucans or xyloglucans and other plant material containing cellulosic parts. The authorized name is endo-1,4-beta-D-glucan 4-glucano hydrolase, but the abbreviated term endoglucanase is used in the present specification. Endoglucanase activity may be determined using carboxymethyl cellulose (CMC) hydrolysis according to the procedure of Ghose, 1987, Pure and Appl. Chem. 59: 257-268 the teaching of which is incorporated herein by reference.

In some embodiments endoglucanases may be derived from a strain of the genus Trichoderma, such as a strain of Trichoderma reesei; a strain of the genus Humicola, such as a strain of Humicola insolens; or a strain of Chrysosporium, preferably a strain of Chrysosporium lucknowense. Suitably the cellulase for use in the present invention may be a Chrysosporium lucknowense cellulase available from Dyadic International USA Inc, e.g. as taught in U.S. Pat. No. 7,892,812, the teachings of which are incorporated herein by reference. In some embodiments the cellulase may be the product of expression of one or more enzyme(s) in a suitable host cell (e.g. a fermentation product).

Suitably, the enzyme composition comprising cellulase activity may be obtainable (e.g. obtained) from Trichoderma, preferably from Trichoderma reesei.

In one embodiment the cellulase for use in the methods and/or uses in accordance with the present invention may be or may comprise LAMINEX® BG2 (available from Danisco A/S Genencor Division).

In one embodiment the cellulose may be a fermentation of Penicillum funiculosum in combination with a fermentation from Trichoderma, e.g. Trichoderma reesei.

In another embodiment the cellulase according to the present invention may be Laminex® Super 3G (available from Danisco A/S Genencor Division).

The enzyme composition comprising cellulase for use in the present invention in preferably one which solubilises as much sediment as possible.

In one embodiment suitably both a cellulase and a mannanase are used in combination with the enzyme that degrades a phospholipid.

The enzyme composition for use in the present invention may comprises cellulase activity together with mannanase activity. In such a situation the enzyme composition suitably may comprise a minimum level of mannanase activity such that when added to the substrate a mannanase concentration of at least 200 MVR/kg substrate is achieved.

In some embodiments the cellulase composition may comprise mannanase side activity. Where the mannanase activity is a side activity it must represent at least 200 MVR/kg substrate. Alternatively a separate mannanase enzyme may be added to a cellulase composition to ensure at least 200 MVR/kg substrate mannanase activity is present in the substrate.

Where the enzyme composition comprises mannanase, preferably the enzyme composition for use in the present invention comprises a minimum level of mannanase activity which when added to the substrate will give a mannanase concentration of at least about 200 MVR/kg substrate.

In one embodiment a xylanase may be used in combination with the enzyme that degrades a phospholipid. Suitably this may be in combination with a cellulase and/or a mannanase.

In one embodiment the xylanase is of microbial origin, such as of fungal origin (e.g., Trichoderma, Meripilus, Humicola, Aspergillus, Fusarium) or from a bacterium (e.g., Bacillus). In some embodiments the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase. Examples of commercial xylanases include Grindamyl Powerbake 930 from Danisco A/S, Denmark or SHEARZYME™ and BIOFEED WHEAT™ from Novozymes A/S, Denmark.

In another embodiment a glucuronidase may be used in combination with the enzyme that degrades a phospholipid. The glucuronidase for use in accordance with the present invention may be one or more selected from: a 1,2-alpha-glucuronidase (E.C. 3.2.1.131), an alpha-glucuronidase (E.C. 3.2.1.139), a beta-glucuronidase (E.C. 3.2.1.31), a glucuronosyldisulfoglucosamine glucuronidase (E.C. 3.2.1.56) or a combination thereof.

As used herein the term “beta-glucuronidase” is synonymous with “beta-glucuronide glucuronohydrolase”.

In another embodiment the glucuronidase may be a 1,2-alpha-glucuronidase (E.C. 3.2.1.131).

In one embodiment the glucuronidase may be an alpha-glucuronidase (E.C. 3.2.1.139).

In another embodiment the glucuronidase may be a beta-glucuronidase (E.C. 3.2.1.31).

In a different embodiment the glucuronidase may be a glucuronosyldisulfoglucosamine (E.C. 3.2.1.56).

In another embodiment a galactanase may be used in combination with the enzyme that degrades a phospholipid. The galactanase may be selected from an exo-galactanase (E.C. 3.2.1.23) or an endo-galactanase (E.C. 3.2.1.89) e.g. an arabinogalactan endo-1,4-beta-galactosidase or a galactan endo-beta-1,3-galactanase (E.C. 3.2.1.181). Arabinogalactan endo-1,4-beta-galactosidase catalyses the endohydrolysis of 1,4-D-galactosidic linkages in arabinogalactans.

The term “xylanase” as used herein refers to an enzyme that is able to hydrolyze the beta-1,4 glycosyl bond in non-terminal beta-D-xylopyranosyl-1,4-beta-D-xylopyranosyl units of xylan or arabinoxylan. Other names include 1,4-beta-D-xylan xylanohydrolase, 1,4-beta-xylan xylanohydrolase, beta-1,4-xylan xylanohydrolase, (1-4)-beta-xylan 4-xylanohydrolase, endo-1,4-beta-xylanase, endo-(1-4)-beta-xylanase, endo-beta-1,4-xylanase, endo-1,4-beta-D-xylanase, endo-1,4-xylanase, xylanase, beta-1,4-xylanase, beta-xylanase, beta-D-xylanase. Xylanases can be derived from a variety of organisms, including plant, fungal (e.g. species of Aspergillus, Penicillium, Disporotrichum, Neurospora, Fusarium, Humicola, Trichoderma, Geosmithia, Talaromyces) or bacterial species (e.g. species of Bacillus, Aeromonas, Streptomyces, Nocardiopsis, Thermomyces) (see for example WO92/17573, WO92/01793, WO91/19782, WO94/21785 which are incorporated herein by reference). In one aspect of the invention, the xylanase used in the methods of the invention is an enzyme classified as EC 3.2.1.8. The official name is endo-1,4-beta-xylanase. The systematic name is 1,4-beta-D-xylan xylanohydrolase. Other names may be used, such as endo-(1-4)-beta-xylanase; (1-4)-beta-xylan 4-xylanohydrolase; endo-1,4-xylanase; xylanase; beta-1,4-xylanase; endo-1,4-xylanase; endo-beta-1,4-xylanase; endo-1,4-beta-D-xylanase; 1,4-beta-xylan xylanohydrolase; beta-xylanase; beta-1,4-xylan xylanohydrolase; endo-1,4-beta-xylanase; beta-D-xylanase. The reaction catalyzed is the endohydrolysis of 1,4-beta-D-xylosidic linkages in xylans.

The mannanase for use in the present invention may be any commercially available mannanase. The mannanase may be an endo-1,4-β-D-mannanase (classified as E.C. 3.2.1.78) or a β-mannosidase (classified as E.C. 3.2.1.25).

In one embodiment preferably the mannanase is an endomannanase, e.g. an endo-1,4-β-D-mannanase. The classification for an endo-1,4-β-D-mannanase (β-mannanase) is E.C. 3.2.1.78.

In one embodiment the mannanase may be a β-mannanase (E.C. 3.2.1.78) from Bacillus.

In one embodiment the mannanase may be a β-mannanase (E.C. 3.2.1.78) from Bacillus lentus or Bacillus subtilis or Bacillus licheniformis.

In one embodiment the enzyme composition may comprise a mannanase (e.g. E.C. 3.2.1.78) produced in Trichoderma, e.g. Trichoderma reesei.

In one embodiment the mannanase may be Mannastar 375 375® (Available commercially from DuPont Industrial Biosciences).

In one embodiment the mannanase may be a β-mannanase (E.C. 3.2.1.78) from Bacillus lentus, e.g. such as the commercial Hemicell® and Hemicell-HT product from ChemGen Corp. (Elanco).

In one embodiment the mannanase may be a Hemicell®-W (a commercial product sold by ChemGen Corp. comprising β-Mannanase (EC 3.2.1.78) from Bacillus lentus and a xylanase (EC 3.2.1.8) from Trichoderma longibrachiatum).

In one embodiment the mannanase may be a 1-mannanase (E.C. 3.2.1.78) from Bacillus licheniformis, such as the β-Mannanase (EC 3.2.1.78) from Bacillus licheniformis sold in CTCzyme—a product sold by CTC BIO Inc.

In one embodiment the mannanase may be Zymanase® (a commercial product sold by ChemGen Corp. comprising a β-Mannanase (EC 3.2.1.78) and a β-glucanase.

In one embodiment the mannanase may be CTCzyme—a product sold by CTC BIO Inc., and comprising a β-Mannanase (EC 3.2.1.78) from Bacillus licheniformis (B. licheniformis gene expressed in B. subtilis).

In one embodiment the mannanase may be a mannanase taught in U.S. Pat. No. 7,846,705, which is incorporated herein by reference.

In one embodiment the enzymes for use in the present invention are suitably thermostable.

In one embodiment the enzyme may be cross-linked with glutaraldehyde in order to improve the enzymes thermostability. For example see the teachings of Schmid et a Adv. Biochemc Eng. 12, p 41,118 1979 and EP0575323B1, which are both incorporated herein by reference. In one embodiment the enzyme or thermostable enzyme is not a genetically modified enzyme.

The term “thermostability” is the ability of an enzyme to resist irreversible inactivation (usually by denaturation) at a relatively high temperature. This means that the enzyme retains a specified amount of enzymatic activity after exposure to an identified temperature over a given period of time.

There are many ways of measuring thermostability. By way of example, enzyme samples maybe incubated without substrate for a defined period of time (e.g. 10 min or 1 to 30 min) at an elevated temperature compared to the temperature at which the enzyme is stable for a longer time (days). Following the incubation at elevated temperature the enzyme sample is assayed for residual activity at the permissive temperature of e.g. 30° C. (alternatively 25-50° C. or even up to 70° C.). Residual activity is calculated as relative to a sample of the enzyme that has not been incubated at the elevated temperature.

Thermostability can also be measured as enzyme inactivation as function of temperature. Here enzyme samples are incubated without substrate for a defined period of time (e.g. 10 min or 1 to 30 min) at various temperatures and following incubation assayed for residual activity at the permissive temperature of e.g. 30° C. (alternatively 25-70° C. or even higher). Residual activity at each temperature is calculated as relative to a sample of the enzyme that has not been incubated at the elevated temperature. The resulting thermal denaturation profile (temperature versus residual activity) can be used to calculate the temperature at which 50% residual activity is obtained. This value is defined as the Tm value.

Even further, thermostability can be measured as enzyme inactivation as function of time. Here enzyme samples are incubated without substrate at a defined elevated temperature (e.g. 76° C.) for various time periods (e.g. between 10 sec and 30 min) and following incubation assayed for residual activity at the permissive temperature of e.g. 30° C. (alternatively 25-70° C. or even higher). Residual activity at each temperature is calculated as relative to an enzyme sample that has not been incubated at the elevated temperature. The resulting inactivation profile (time versus residual activity) can be used to calculate the time at which 50% residual activity is obtained. This is usually given as T1/2.

These are examples of how to measure thermostability. Thermostability can also be measured by other methods. Preferably thermostability is assessed by use of the “Assay for measurement of thermostability” as taught herein.

In contradistinction to thermostability, thermoactivity is enzyme activity as a function of temperature. To determine thermoactivity enzyme samples may be incubated (assayed) for the period of time defined by the assay at various temperatures in the presence of substrate. Enzyme activity is obtained during or immediately after incubation as defined by the assay (e.g. reading an OD-value which reflects the amount of formed reaction product). The temperature at which the highest activity is obtained is the temperature optimum of the enzyme at the given assay conditions. The activity obtained at each temperature can be calculated relative to the activity obtained at optimum temperature. This will provide a temperature profile for the enzyme at the given assay conditions.

The thermostability of an enzyme (e.g. a fiber degrading enzyme) for use in accordance with the present invention may be determined using the “Assay for measurement of thermostability” (see below).

### “Assay for Measurement of Thermostability”

The thermal denaturation profiles of the enzyme is measured by diluting and pre-incubating the enzyme samples in 25 mM acetate buffer, pH 4.5 for 10 min at varying temperatures (60, 65, 70, 75, 80, 85 and 90° C., respectively) and subsequently measuring the residual activity of the enzyme when tested in the “Beta-Glucanase Activity Assay” described herein.

In the assay, activity measured without pre-incubation is set to 100% and the residual activity of an enzyme at each temperature is calculated as relative to this. Tm value is calculated from the thermal denaturation profiles as the temperature at which 50% residual activity is obtained.

In one embodiment, an enzyme is considered to be thermostable in accordance with the present invention if it has a Tm value of more than 70° C., wherein the Tm value is the temperature at which 50% residual activity is obtained after 10 min incubation. This Tm value may be measured in accordance with the assay for measurement of thermostability as taught herein.

In one embodiment, an enzyme is considered to be thermostable in accordance with the present invention if it has a Tm value of more than 75° C., wherein the Tm value is the temperature at which 50% residual activity is obtained after 10 min incubation. This Tm value may be measured in accordance with the assay for measurement of thermostability as taught herein.

In one embodiment, an enzyme is considered to be thermostable in accordance with the present invention if it has a Tm value of more than 80° C., wherein the Tm value is the temperature at which 50% residual activity is obtained after 10 min incubation. This Tm value may be measured in accordance with the assay for measurement of thermostability as taught herein.

In one embodiment the enzyme according to the present invention is not Celluclast® (by Novozymes, A/S).

In one embodiment, the enzyme does not comprise pectinase activity.

In one embodiment the enzyme or thermostable enzyme for use in the methods and/or uses of the present invention may be immobilized.

The term “immobilized” as used herein means that the enzyme or enzyme is fixed in position and its movement impeded but the activity of the enzyme or thermostable enzyme is not substantially altered by such immobilization. Suitably, an immobilized enzyme or thermostable enzyme may retain at least 50% of its activity when compared to a non-immobilized enzyme or thermostable enzyme. Suitably it may retain at least about 50%, 60%, 70%, 80%, 90% or 95% of its activity when compared to a non-immobilized enzyme or thermostable enzyme. An “immobilized” enzyme or thermostable enzyme may be fixed to a surface. This may be achieved by any known means within the art which do not substantially alter the activity of the enzyme or thermostable enzyme. Suitably, the enzyme or thermostable enzyme may be immobilized by cross-linking (e.g. cross-linking to a surface). By way of example the enzyme or thermostable enzyme may be cross-linked using glutaraldehyde (Migneault et al BioTechniques 37: 790:802 (November 2004).

### Enzyme Activity Assays

### Cellulase Activity Assay: By the CMC-DNS Procedure:

The assay of cellulase activity (e.g. endo-1,4-β-glucanase activity) is based on the enzymatic hydrolysis of the 1,4-β-D-glucosidic bonds in carboxymethylcellulose (CM-Cellulose 4M, Megazyme Ltd) a β-1,4-glucan. The enzyme is diluted in ddH₂0 and 0.25 ml enzyme solution added to 1.75 ml substrate (1,5% CMC in 0.2M sodium acetate buffer, pH 5.0) at 50′C. After 10 min of incubation a 2 ml 1% 3,5-Dinitrosalicylic acid (DNS) solution is added and the sample is placed in boiling water bath for 5 min. The products of the reaction (β-1,4 glucan oligosaccharides) are determined colorimetrically at 540 nm by measuring the resulting increase in reducing groups reacting with the DNS. Enzyme activity is calculated from the relationship between the concentration of reducing groups, as glucose equivalents, and absorbance using a glucose standard in the range 0.125-0.5 mg/ml. One unit of cellulase activity is defined as the amount of enzyme which produces 1 μmole glucose equivalents per minute under assay conditions.

In one embodiment a cellulase in accordance with the present invention is a cellulase which reduces the amount of dry sediment (dry matter) by at least 20% when 25000 CMC-DNS/kg substrate is added to a palm fruit, a portion thereof or a palm fruit extract and incubated for 1 hr at 50° C. and the sludge dry matter is analysed by the following method:

After incubation the sample was placed in at water bath at 95 degrees for 10 minutes to stop the enzyme reaction, and transferred to a tarred 50 ml centrifuge tube.

The sample was centrifuged at 4180 rcf and 60° C. for 10 minutes.

The upper oil layer was removed, and remaining water phase was discharged. 30 ml water at 50° C. was added to each tube. The sample was centrifuged at 4180 rcf and 60 degree C. for 10 minutes.

The water phase was removed and the side of the tube was wiped with a tissue to remove residual oil on the inside of the tube. The wet sediment was scaled, frozen and freeze dried.

Weight of the dry sediment was determined after freeze drying.

### Pectinase and Mannanase Activity (Pvr Uig and Mvr Uig) Assays Measured by Viscosity Reduction Procedure:

25, 50, 75 and 100 μL of an enzyme sample diluted in ddH₂O is added to hydrocolloid solution (0.5% Grindsted GUAR 250 pH 6.7 or 1.4% Pectin SY200 pH 4 in Citric acid-Sodium phosphate buffer) and incubated 19 hours at 40° C. The hydrocolloid is cleaved by the enzyme to oligosaccharides, thereby creating a drop in viscosity of the solution. Following this, samples are tempered for 20 minutes on ice before measuring viscosity at 0° C. using a Viscoman pipette (Gilson, Inc. USA). The viscosity reduction is calculated as the viscosity of a sample with addition of enzyme relative to viscosity of sample without enzyme. The viscosity reduction is plotted against LN (μL dosage in substrate) and should be linear within relative viscosity of 0.1 to 0.85. Activity of the sample in U/g is calculated using the regression line. Pectinase viscosity reduction (PVR) and Mannanase viscosity reduction (MVR) units are defined as the amount of enzyme that will degrade the hydrocolloid substrate solution to a 50% (0.5) viscosity reduction in 19 hours of incubation at 40° C.

Where the enzyme composition comprises mannanase, preferably the enzyme composition for use in the present invention comprises a minimum level of mannanase activity which when added to the substrate will give a mannanase concentration of at least about 200 MVR/kg substrate.

Where the enzyme composition comprises pectinase, preferably the enzyme composition for use in the present invention comprises a minimum level of pectinase activity which when added to the substrate will give a mannanase concentration of at least about 9 PVR/kg substrate.

In one embodiment the enzyme composition for use in the present invention comprises low or no protease. In other words preferably there is no or only very low levels of protease activity in the reaction admixture during incubation.

Protease Activity (PU) Assay—with Sulfanilamide-Azocasein

The azocasein assay is based on hydrolyses of the azocasein which releases the azo dyed peptide in the supernatant where it is detected at 450 nm. These peptides cannot precipitate by the addition of acid, as against non hydrolysed azocasein, which precipitates.

Substrate: 0.25% Azocasein (Sigma A2765) dissolved in 50 mM sodium-citrate buffer pH 6.

Procedure: 100 μl enzyme solution is incubated with 250 μl substrate for 30 minutes at 40° C. 50 μl 2M Trichloracetic acid is added, and the sample is centrifuged at 10000 rcf for 5 minutes. 195 μl supernatant is transferred to a microtiter filterplate (0.2 μm PVDF Hydrophilic membrane) and 85 μl 1M NaOH is added. The sample is filtered into another microtiter plate by centrifugation at 2400 rcf for 2 minutes. OD 450 of the filtrated sample is read.

The activity of the enzyme sample is measured based on a calibration curve obtained by analyzing different dilutions of a commercial Protease, Protex 14L (Standardized to 150 PU/ml) and construction of a calibration curve of OD₄₅₀ as a function of PU/g.

In one embodiment preferably the enzyme composition for use in the present invention has not more than about 0.1 PU/ml.

In one embodiment preferably the enzyme composition for use in the present invention has not more than about 0.04 PU/ml.

### Xylanase Activity Assay

To 1.0 ml aliquots of assay buffer (0.1 M NaAc, pH 5.0) is added 25 μl, 50 μl, 75 μl and 100 μl of enzyme solution and the mixtures are equilibrated at 40.00° C. for 5 minutes. One XylaZyme tablet (Megazyme cat no. T-XYZ100) (e.g. containing AZCL-arabinoxylan (wheat)) is added to each test tube and the test tubes must not be stirred. After exactly 10 minutes incubation 10 ml stop solution (1% (w/v) Tris(hydroxymethyl)-aminomethane) is added. The test tubes are stirred and the solutions are filtered through Whatman No. 1 filter paper.

The absorbance at 590 nm of standard and test samples is measured against a blank sample without enzyme. The concentrations of standard and sample enzymes are adjusted so that the optical densities (OD) at 590 nm are within the range 0.2-1.1.

The Standard enzyme is xylanase from Aspergillus niger, Megazyme cat. no. E-XYAN4.

The OD₅₉₀'s for standard and test enzymes are plotted against the volumes of enzyme solution added. The best curve fit is found using linear regression. The volumes of standard (Vₛₜ) and test enzymes (Vₜ) corresponding to an OD₅₉₀ of 0.7 are calculated.

$$XU \text{/} g = \frac{{ACT}_{st}^{*} {V}_{st}^{*} {D}_{t}^{*} {A}_{st}}{{V}_{t}^{*} {D}_{st}^{*} {A}_{t}}$$

where ACTₛₜ=activity of standard enzyme preparation, XU/g
Dₜ=dilution of test sample, ml
Dₛₜ=dilution of standard enzyme, ml
Aₛₜ=amount of standard enzyme, g
Aₜ=amount of test sample in g
Vₛₜ=volume of standard enzyme read on x-axis, μl
Vₜ=volume of test sample read on x-axis, μl

An enzyme is a xylanase in accordance with the present invention if in the Xylanase Activity Assay herein it has at least 100 Units/ml

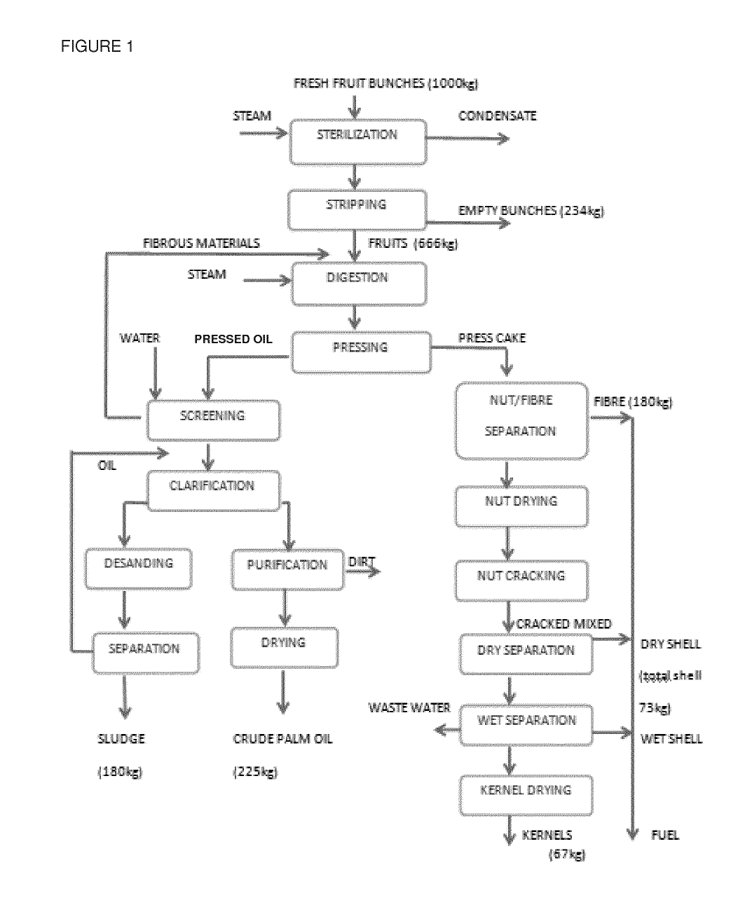

In one embodiment the separation of the crude oil from the sludge may be carried out by clarification. “Clarification” as used herein means using gravity to allow the oil to settle out of the sludge. During clarification the oil becomes “clear”. Clarification may take place between approximately 90 and 95° C. Typically one would leave the admixture for 1-3 hours to allow for the clarification process to occur. Large settling tanks (so-called vertical clarifiers) may be used in which the crude oil settles out of the sludge. The oil may be skimmed off the sludge. With the sludge typically coming out of the bottom of a vertical clarifier (this sludge is typically called the “underflow”). “Sludge” as used herein is a heavy fraction of pressed palm fruit extract comprising oil, water and some organic material that has a tendency to settle out of pressed palm fruit extract. Sludge may be obtained post clarification, for example following separation as shown in FIG. 1.

Mechanisms may be employed to improve recovery of residual oil from the settled sludge (e.g. post clarification). For example a centrifuge may be used to extract further oil from the sludge. Typically therefore the sludge may be sent to a centrifuge. Alternatively a decanter may be used in combination with clarification. For example, 3-phase decanters available as “Westfalia Separator® topd 3-Phase Decanter” may be used to separate the sludge from the clarification process into 3-phases: an oil-phase, solids, and virtually oil-free waste water. Alternatively, nozzle-type separates such as the one from GEA Westfalia may be used which separate the sludge from the clarification step into three phases: palm oil, solid concentrate and water.

The separation of the crude oil from the sludge may be carried out by centrifugation or decanting (e.g. without clarification). 3-phase decanters available as “Westfalia Separator® topd 3-Phase Decanter” can be used directly with enzyme treated pressed palm fruit extract without clarification. This may have the advantage of shorter processing time, smaller dimensions of the process lines. Also the risk of oxidation of the crude oil is less significant compared to the process using vertical clarifiers. Such a decanter separates the sludge into 3-phases an oil-phase, a dry solids cake and virtually oil-free waste water. Centrifugation may be used during or after the decanting process.

The method may further comprise purifiers downstream of the separation step (e.g. downstream of the clarification and/or decanting stage). The purifiers may remove even extremely small amounts of oil left in the effluent.

The method may further comprise a desanding step—which step removes sand to avoid erosion problems caused thereby. The sand may be removed by any method known to one skilled in the art. As the skilled person will appreciate, a multicyclone system may be used to separate the sand. Typically the desanding step may occur prior to the centrifugation step.

The pressed palm fruit extract may be prepared by sterilizing the fresh palm fruit bunches, stripping the fruit from the bunches, optionally digestion of the fruit and pressing. The digestion may take place in a digester. During the digestion, the palm fruit is disintegrated and oil released from the fiber. After digestion, the crude oil is separated from the fiber by pressing the digested fruits.

The method of the present invention may include one or more of the following steps after pressing of the palm fruit:
Removing large solid particulates/course fibres from the pressed palm fruit extract—e.g. this may be achieved by passing the pressed palm fruit extract through a screen which may be vibrating;
Adding extra water to the pressed palm fruit extract—e.g. to reduce viscosity and enhance separation of crude oil from the other components.

These additional steps may be carried out before clarification and/or decanting.

In one embodiment the enzyme for use in the present invention is admixed with one or more palm fruit(s) or a portion thereof prior to pressing. Suitably this palm fruit(s) or portion thereof is/are admixed with the enzyme during a digestion step. In one embodiment the palm fruit or portion thereof may be admixed with the enzyme in a digester. In another embodiment the enzyme for use in the present invention may be admixed with digested palm fruit or portion thereof prior to pressing.

The term “or a portion thereof” in relation to the palm fruit(s) as used herein means the part of the palm fruits that are remaining post stripping the fruit from the bunches.

In one embodiment the palm fruit or a portion thereof or the palm fruit extract may be one having a diglyceride content of about 3% w/w to about 8% w/w.

In another embodiment the enzyme for use in the present invention may be admixed with pressed palm fruit extract prior to separating the crude palm oil from the sludge (e.g. by clarification or decanting or centrifugation).

In one embodiment the enzyme for use in the present invention may be admixed with pressed palm fruit extract during the separation step, e.g. when the crude palm oil is being separated from the sludge (e.g. by clarification or centrifugation). In particular, in one embodiment the enzyme for use in the present invention may be admixed with pressed palm fruit extract during clarification (e.g. in the clarifying tank).

The term “pressed palm fruit extract” is also known in the industry as “pressed liquid” or “pressed oil”. For the avoidance of doubt the term pressed palm fruit extract is not a crude palm oil or semi-crude palm oil and has typically not been water or enzymatic degummed. A crude palm oil is the resulting oil that is purified and dried prior to shipment to refining/degumming plants.

Crude palm oil has three main components, which are a mixture of oil and water, oil-in-water emulsions and water-in-oil emulsions. A crude palm oil is typically separated into pure oil and sludge in a clarifying station (Stork et al 1996 (which is referenced in Master Thesis: SEPARATION TEHCNIQUE OF CRUDE PALM OIL AT CLARIFICATION AREA VIA OPTIMUM PARAMETERS NURULHUDA BINTI KASIM A thesis submitted in fulfillment of the requirement for the award of the degree of Bachelor of Chemical Engineering Faculty of Chemical and Natural Resources Engineering Universiti Malaysia Pahang APRIL 2009) the content of which is incorporated herein by reference) An approximate average composition of screw pressed crude palm oil might be 64% oil, 24% water and 12% non-oil solid (Maycock et al (1987) Palm Oil Factory Process Handbook Part 1, PORIM, Bangi the content of which is incorporated herein by reference). Without wishing to be bound by theory, examination of sludge samples typically reveals the presence of oil droplets of sizes varying from less than 1 μm. The difference in specific gravity between sludge oil is practically contestant at 0.1 throughout the temperature range from 40° C. to 100° C. (Stork et al 1996). The largest solid impurity to be separated is the fibre and the smallest is the cellular debris. Again without wishing to be bound by theory due to the high ratio of solids to oil and the low ratio of water to solids in the crude oil from a screw press, water is added in order to enhance the settling efficiency. Without wishing to be bound by theory it is believed that viscosity increases with the amount of water added up to 50% dilution and beyond this point, the viscosity continuously falls with higher dilution but less steeply.

The pressed palm fruit extract in accordance with the present invention is a composite containing: oil, water and non-oil solids (comprising lignin, carbohydrates, proteins and inorganic solids). The mixture is an emulsion of oil-in-water from which the oil is separated.

The pressed palm fruit extract comprises typically approximately 64% oil, 24% water, 12% non-oil solids (comprising lignin, carbohydrate, proteins and inorganic solids). It will be known to one skilled in the art the levels of each of these components in the pressed palm fruit extract may vary depending on the starting palm fruits used, which vary according to location and year of harvest as well as, for example, the amount of water in the fruit. Without wishing to be bound by theory, the amount of water in the fruit may vary depending on the whether the climate conditions are wet or dry during harvest, for example. However, the pressed palm fruit extract according to the present invention typically has between 60 and 85% oil. However, the person skilled in the art will further appreciate that the oil content in the pressed palm fruit extract may vary, e.g. when the water content is increased. Thus, in some embodiments, e.g. where steam is injected into the digester to raise temperatures the water content may be higher and the oil content lower. The pressed palm fruit extract according to the present invention may have a relatively high level of non-oil solids (typically being in the region of 5-15% w/w). In some instances the level of non-oil solids may be at least more than 5% w/w of the pressed palm fruit extract.

Typically 7-10% w/w (e.g. 8.5% w/w) of the total non-oil solids in pressed palm fruit extract is protein. Therefore in one embodiment the present invention relates to a palm fruit extract (e.g. a pressed palm fruit extract) that comprises at least 7% w/w (suitably at least 8.4% w/w) total non-oil solids.

In some embodiments the present invention relates to a palm fruit extract (e.g. a pressed palm fruit extract) that comprises at from at least about 6.5% to about 12% w/w total non-oil solids.

Approximately 0.5-2% w/w (typically approximately 1% w/w) of the total pressed palm extract is protein. Therefore in one embodiment the present invention relates to a palm fruit extract (e.g. a pressed palm fruit extract that comprises at least 0.5%, preferably at least 1% w/w protein.

For the avoidance of doubt, crude palm extract contains about 99% w/w oil because almost all of the non-oil solids have been removed. In one embodiment the non-hydratable phosphorus content of the palm fruit extract (e.g. the pressed palm fruit extract) is less than ppm, preferably less than 30 ppm.

In some embodiments the non-hydratable phosphorus content may be considered to be equivalent to non-hydratable phospholipids. Thus, in some embodiments, the non-hydratable phosphorus in non-hydratable phospholipids content of the palm fruit extract (e.g. the pressed palm fruit extract) may be less than 45 ppm, preferably less than 30 ppm.

The incubation of the admixed enzyme and palm fruit extract in accordance with the present invention may be carried out at about 45° C. to about 95° C.

In one embodiment the incubation may occur at about 50° C. to about 90° C. In another embodiment the incubation may occur at about 50° C. to about 85° C. Suitably incubation may occur at about 50° C. to about 60° C.

Suitably, the incubation may occur at about 50° C.

Incubation of the enzyme and palm fruit extract in accordance with the present invention may occur for between about 15 minutes to about 6 hours.

In one embodiment incubation occurs for between about 2 hours to about 6 hours. In another embodiment incubation occurs for between about 2 hours to about 4 hours.

Suitably admixing occurs for about 2 hours.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Amino acids are referred to herein using the name of the amino acid, the three letter abbreviation or the single letter abbreviation.

The term “protein”, as used herein, includes proteins, polypeptides, and peptides.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an enzyme” includes a plurality of such candidate agents and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

The term “transferase” as used herein is interchangeable with the term “lipid acyltransferase”. Suitably, the lipid acyltransferase as defined herein catalyses a transesterification.

The term “interesterification” refers to the enzymatic catalysed transfer of acyl groups between a lipid donor and lipid acceptor, wherein the lipid donor is not a free acyl group.

The term “transesterification” as used herein means the enzymatic catalysed transfer of an acyl group from a lipid donor (other than a free fatty acid) to an acyl acceptor (other than water).

As used herein, the term “alcoholysis” refers to the enzymatic cleavage of a covalent bond of an acid derivative by reaction with an alcohol ROH so that one of the products combines with the H of the alcohol and the other product combines with the OR group of the alcohol.

As used herein, the term “alcohol” refers to an alkyl compound containing a hydroxyl group.

As used herein, the term “hydrolysis” refers to the enzymatic catalysed transfer of an acyl group from a lipid to the OH group of a water molecule.

### Lipid Acyltransferase

The nucleotide sequence encoding a lipid acyl transferase for use in any one of the methods and/or uses of the present invention may encode a natural lipid acyl transferase or a variant lipid acyl transferase.

The lipid acyl transferase for use in any one of the methods and/or uses of the present invention may be a natural lipid acyl transferase or a variant lipid acyl transferase.

For instance, the nucleotide sequence encoding a lipid acyl transferase for use in the present invention may be one as described in WO 2004/064537, WO 2004/064987, WO 2005/066347, WO 2006/008508, WO 2009/024862, WO2011/061657, or WO2011/061657. These documents are incorporated herein by reference.

The term “lipid acyl transferase” as used herein preferably means an enzyme that has acyltransferase activity (generally classified as E.C. 2.3.1.x, for example 2.3.1.43), whereby the enzyme is capable of transferring an acyl group from a lipid to one or more acceptor substrates, such as one or more of the following: a sterol; a stanol; a carbohydrate; a protein; a protein subunit; a sugar alcohol, such as ascorbic acid and/or glycerol—preferably glycerol and/or a sterol, such as cholesterol.

Preferably, the lipid acyl transferase for use in any one of the methods and/or uses of the present invention is a lipid acyltransferase that is capable of transferring an acyl group from a phospholipid (as defined herein) to a sugar alcohol, such as ascorbic acid and/or glycerol and/or a sterol, preferably glycerol or a sterol, most preferably a sterol (e.g. cholesterol).

Suitably, the lipid acyl transferase for use in any one of the methods and/or uses of the present invention is a lipid acyltransferase that is capable of transferring an acyl group from a phospholipid (as defined herein) to a phytosterol and/or a phytostanol. Suitably, the lipid acyttransferase may be one that is capable of transferring an acyl group from a phospholipid (as defined herein) to a phytosterol.

Preferably the phytosterol and/or phytostanol comprises one or more of the following structural features:
i) a 3-beta hydroxy group or a 3-alpha hydroxy group; and/or
ii) A:B rings in the cis position or A:B rings in the trans position or C₅-C₆ is unsaturated.

In one embodiment, preferably the phytosterol is selected from the group consisting of one or more of the following: alpha-sitosterol, beta-sitosterol, stigmasterol, ergosterol, campesterol, 5,6-dihydrosterol, brassica sterol, alpha-spinasterol, beta-spinasterol, gamma-spinasterol, deltaspinasterol, fucosterol, dimosterol, ascosterol, serebisterol, episterol, anasterol, avenasterol, clionasterol, hyposterol, chondrillasterol, desmosterol, chalinosterol, poriferasterol, clionasterol, sterol glycosides, and other natural or synthetic isomeric forms and derivatives.

In one embodiment, preferably the phytostanol is selected from the group consisting of one or more of the following: alpha-sitostanol, beta-sitostanol, stigmastanol, ergostanol, campestanol, 5,6-dihydrostanol, brassica stanol, alpha-spinastanol, beta-spinastanol, gamma-spinastanol, deltaspinastanol, fucostanol, dimostanol, ascostanol, serebistanol, epistanol, anastanol, avenastanol, clionastanol, hypostanol, chondrillastanol, desmostanol, chalinostanol, poriferastanol, clionastanol, stanol glycosides, and other natural or synthetic isomeric forms and derivatives.

Suitably, phytostanols for use in the present invention may be obtained from hydrogenation of sterols (see U.S. Pat. No. 6,866,837 for example).

For some aspects the “acyl acceptor” according to the present invention may be any compound comprising a hydroxy group (—OH), such as for example, polyvalent alcohols, including glycerol; sterols; stanols; carbohydrates; hydroxy acids including fruit acids, citric acid, tartaric acid, lactic acid and ascorbic acid; proteins or a sub-unit thereof, such as amino acids, protein hydrolysates and peptides (partly hydrolysed protein) for example; and mixtures and derivatives thereof. Preferably, the “acyl acceptor” according to the present invention is not water. Preferably, the “acyl acceptor” according to the present invention is a sugar alcohol, such as a polyol, most preferably glycerol. For the purpose of this invention ascorbic acid is also considered a sugar-alcohol.

The acyl acceptor is preferably not a monoglyceride.

The acyl acceptor is preferably not a diglyceride.

In one aspect, the lipid acytransferase for use in any one of the methods and/or uses of the present invention is a lipid acyltransferase that may, as well as being able to transfer an acyl group from a lipid to glycerol, additionally be able to transfer the acyl group from a lipid to one or more of the following: a carbohydrate, a protein, a protein subunit, sterol and/or a stanol, preferably it is capable of transferring to both a sugar alcohol, such as ascorbic acid and/or glycerol, most preferably a sterol such as cholesterol, and/or plant sterols/stanols.

In some aspects, the lipid acyltransferase for use in any one of the methods and/or uses of the present invention is a lipid acyltransferase that is capable of esterifying at least about 10%, more preferably at least about 20%, 30%, 40%, 50%, 60% or 70% of the acyl acceptor. Preferably, the lipid substrate upon which the lipid acyltransferase acts is one or more of the following lipids: a phospholipid, such as a lecithin, e.g. phosphatidylcholine and/or phophatidylethanolamine.

This lipid substrate may be referred to herein as the “lipid acyl donor”. The term lecithin as used herein encompasses phosphatidylcholine, phosphatidylethanolamine, phosphatidylinositol, phosphatidylserine and phosphatidylglycerol.

For some aspects, preferably the lipid acyl transferase for use in any one of the methods and/or uses of the present invention is a lipid acyltransferase that is incapable, or substantially incapable, of acting on a triglyceride and/or a 1-monoglyceride and/or 2-monoglyceride.

For some aspects, preferably the lipid acytransferase for use in any one of the methods and/or uses of the present invention is a lipid acyltransferase that does not exhibit triacylglycerol lipase activity (E.C. 3.1.1.3) or does not exhibit significant triacylglycerol lipase activity (E.C. 3.1.1.3). The term “does not exhibit significant triacylglycerol lipase activity” means that it has preferably less than 2.5 LUS/mg of enzyme, more preferably less than 2.0 LUS/mg of enzyme, as determined using the titrimetric assay modified to sunflower oil and pH 5.5 instead of olive oil and pH 6.5 as detailed below.

Triacylglycerol activity can be measured using a titrimetric assay according to Food Chemical Codex (3^{rd} Ed., 1981, pp 492-493) modified to sunflower oil and pH 5.5 instead of olive oil and pH 6.5. The lipase activity is measured as LUS (lipase units sunflower) where 1 LUS is defined as the quantity of enzyme which can release 1 μmol of fatty acids per minute from sunflower oil under the assay conditions.

Suitably, the lipid acyltransferase for use in any one of the methods and/or uses of the present invention is a lipid acyltransferase that may exhibit one or more of the following phospholipase activities: phospholipase A2 activity (E.C. 3.1.1.4) and/or phospholipase A1 activity (E.C. 3.1.1.32). The lipid acyl transferase may also have phospholipase B activity (E.C 3.1.1.5).

Suitably, for some aspects the lipid acyltransferase may be capable of transferring an acyl group from a phospholipid to a sugar alcohol, preferably glycerol and/or ascorbic acid.

Suitably, for some aspects the lipid acyltransferase may be capable of transferring an acyl group from a phospholipid to a stanol and/or sterol, preferably cholesterol.

For some aspects, preferably the lipid acyltransferase for use any one of the methods and/or uses of the present invention encodes a lipid acyltransferase that is capable of transferring an acyl group from a phospholipid to a sterol and/or a stanol to form at least a sterol ester and/or a stanol ester.

The lipid acyttransferase may be capable of transferring an acyl group from a lipid to a polyol such as glycerol, and/or a sterol such as cholesterol or plant sterol/stanols. Thus, in one embodiment the “acyl acceptor” according to the present invention may be glycerol and/or cholesterol or plant sterol/stanols.

In some aspects, the lipid acyltransferase for use in any one of the methods and/or uses of the present invention may comprise a GDSx motif and/or a GANDY motif.

Preferably, the lipid acyltransferase enzyme is characterised as an enzyme which possesses acyltransferase activity and which comprises the amino acid sequence motif GDSX, wherein X is one or more of the following amino acid residues L, A, V, I, F, Y, H, Q, T, N, M or S.

Suitably, the nucleotide sequence encoding a lipid acyltransferase or lipid acyltransferase for use in any one of the methods and/or uses of the present invention may be obtainable, preferably obtained, from an organism from one or more of the following genera: Aeromonas, Streptomyces, Saccharomyces, Lactococcus, Mycobacterium, Streptococcus, Lactobacillus, Desulfitobacterium, Bacillus, Campylobacter, Vibrionaceae, Xylella, Sulfolobus, Aspergillus, Schizosaccharomyces, Listeria, Neisseria, Mesorhizobium, Ralstonia, Xanthomonas and Candida. Preferably, the lipid acyltransferase is obtainable, preferably obtained, from an organism from the genus Aeromonas.

In some aspects of the present invention, the nucleotide sequence encoding a lipid acyltransferase for use in any one of the methods and/or uses of the present invention encodes a lipid acyltransferase that comprises an aspartic acid residue at a position corresponding to N-80 in the amino acid sequence of the Aeromonas hydrophila lipid acyltransferase shown as SEQ ID No. 34.

In some aspects of the present invention, the lipid acyltransferase for use in any one of the methods and/or uses of the present invention is a lipid acyltransferase that comprises an aspartic acid residue at a position corresponding to N-80 in the amino acid sequence of the Aeromonas hydrophila lipid acyltransferase shown as SEQ ID No. 34.

In addition or in the alternative, the nucleotide sequence encoding a lipid acyltransferase for use in any one of the methods and/or uses of the present invention encodes a lipid acyltransferase that may comprise the amino acid sequence shown as SEQ ID No. 16, or an amino acid sequence which has 75% or more homology thereto. Suitably, the nucleotide sequence encoding a lipid acyltransferase encodes a lipid acyltransferase that may comprise the amino acid sequence shown as SEQ ID No. 16.

In addition or in the alternative, the nucleotide sequence encoding a lipid acyltransferase for use in any one of the methods and/or uses of the present invention encodes a lipid acyltransferase that may comprise the amino acid sequence shown as SEQ ID No. 68, or an amino acid sequence which has 75% or more homology thereto. Suitably, the nucleotide sequence encoding a lipid acyltransferase encodes a lipid acyltransferase that may comprise the amino acid sequence shown as SEQ ID No. 68.

In one embodiment the lipid acyltransferase for use in any one of the methods and/or uses of the present invention has an amino acid sequence shown in SEQ ID No. 16 or SEQ ID No. 68, or has an amino acid sequence which has at least 75% identity therewith, preferably at least 80%, preferably at least 85%, preferably at least 95%, preferably at least 98% identity therewith.

In one embodiment the lipid acyltransferase for use in any one of the methods and/or uses of the present invention has an amino acid sequence shown in SEQ ID No. 68, or has an amino acid sequence which has at least 75% identity therewith, preferably at least 80%, preferably at least 85%, preferably at least 95%, preferably at least 98% identity therewith.

In one embodiment the lipid acyltransferase for use in any one of the methods and/or uses of the present invention has an amino acid sequence shown in SEQ ID No. 121, or has an amino acid sequence which has at least 75% identity therewith, preferably at least 80%, preferably at least 85%, preferably at least 95%, preferably at least 98% identity therewith. In another embodiment the lipid acyltransferase for use in any one of the methods and/or uses of the present invention has an amino acid sequence shown in SEQ ID No. 122, or has an amino acid sequence which has at least 75% identity therewith, preferably at least 80%, preferably at least 85%, preferably at least 95%, preferably at least 98% identity therewith.

In one embodiment the lipid acyltransferase for use in any one of the methods and/or uses of the present invention has an amino acid sequence shown in SEQ ID No. 123, or has an amino acid sequence which has at least 75% identity therewith, preferably at least 80%, preferably at least 85%, preferably at least 95%, preferably at least 98% identity therewith.

Preferably, the lipid acyltransferase enzyme may be characterised using the following criteria:
the enzyme possesses acyl transferase activity which may be defined as ester transfer activity whereby the acyl part of an original ester bond of a lipid acyl donor is transferred to an acyl acceptor, preferably glycerol or cholesterol, to form a new ester; and
the enzyme comprises the amino acid sequence motif GDSX, wherein X is one or more of the following amino acid residues L, A, V, I, F, Y, H, Q, T, N, M or S.

Preferably, X of the GDSX motif is L or Y. More preferably, X of the GDSX motif is L. Thus, preferably the enzyme according to the present invention comprises the amino acid sequence motif GDSL.

The GDSX motif is comprised of four conserved amino acids. Preferably, the serine within the motif is a catalytic serine of the lipid acyl transferase enzyme. Suitably, the serine of the GDSX motif may be in a position corresponding to Ser-16 in Aeromonas hydrophila lipid acyttransferase enzyme taught in Brumlik & Buckley (Journal of Bacteriology April 1996, Vol. 178, No. 7, p 2060-2064).

To determine if a protein has the GDSX motif according to the present invention, the sequence is preferably compared with the hidden markov model profiles (HMM profiles) of the pfam database in accordance with the procedures taught in WO 2004/064537 or WO 2004/064987, incorporated herein by reference.

Preferably the lipid acyl transferase enzyme can be aligned using the Pfam00657 consensus sequence (for a full explanation see WO 2004/064537 or WO 2004/064987).

Preferably, a positive match with the hidden markov model profile (HMM profile) of the pfam00657 domain family indicates the presence of the GDSL or GDSX domain according to the present invention.

Preferably when aligned with the Pfam00657 consensus sequence the lipid acyltransferase for use in the methods or uses of the invention may have at least one, preferably more than one, preferably more than two, of the following, a GDSx block, a GANDY block, a HPT block. Suitably, the lipid acyltransferase may have a GDSx block and a GANDY block. Alternatively, the enzyme may have a GDSx block and a HPT block. Preferably the enzyme comprises at least a GDSx block. See WO 2004/064537 or WO 2004/064987 for further details.

Preferably, residues of the GANDY motif are selected from GANDY, GGNDA. GGNDL, most preferably GANDY.

Preferably, when aligned with the Pfam00657 consensus sequence the enzyme for use in the methods or uses of the invention have at least one, preferably more than one, preferably more than two, preferably more than three, preferably more than four, preferably more than five, preferably more than six, preferably more than seven, preferably more than eight, preferably more than nine, preferably more than ten, preferably more than eleven, preferably more than twelve, preferably more than thirteen, preferably more than fourteen, of the following amino acid residues when compared to the reference A. hydrophilia polypeptide sequence, namely SEQ ID No. 1: 28His. 29His, 30His, 31His, 32Gly, 33Asp, 34Ser, 35His, 130His, 131Gly, 132His, 133Asn, 134Asp, 135His, 309His. The pfam00657 GDSX domain is a unique identifier which distinguishes proteins possessing this domain from other enzymes.

The pfam00657 consensus sequence is presented in FIG. 5 as SEQ ID No. 2. This is derived from the identification of the pfam family 00657, database version 6, which may also be referred to as pfam00657.6 herein.

The consensus sequence may be updated by using further releases of the pfam database (for example see WO 2004/064537 or WO 2004/064987).

In one embodiment, the lipid acyltransferase enzyme for use in any one of the methods and/or uses of the present invention is a lipid acyltransferase that may be characterised using the following criteria:
(i) the enzyme possesses acyltransferase activity which may be defined as ester transfer activity whereby the acyl part of an original ester bond of a lipid acyl donor is transferred to acyl acceptor, preferably glycerol or cholesterol, to form a new ester, preferably monoglyceride or cholesterol ester respectfully;
(ii) the enzyme comprises the amino acid sequence motif GDSX, wherein X is one or more of the following amino acid residues L, A, V, I, F, Y, H, Q, T, N, M or S;
(iii) the enzyme comprises His-309 or comprises a histidine residue at a position corresponding to His-309 in the Aeromonas hydrophila lipid acyltransferase enzyme shown in FIGS. 4 and 6 (SEQ ID No. 1 or SEQ ID No. 3).

Preferably, the amino acid residue of the GDSX motif is L.

In SEQ ID No. 3 or SEQ ID No. 1 the first 18 amino acid residues form a signal sequence. His-309 of the full length sequence, that is the protein including the signal sequence, equates to His-291 of the mature part of the protein, i.e. the sequence without the signal sequence.

In one embodiment, the lipid acyl transferase enzyme for use any one of the methods and uses of the present invention is a lipid acyltransferase that comprises the following catalytic triad: Ser-34, Asp-306 and His-309 or comprises a serine residue, an aspartic acid residue and a histidine residue, respectively, at positions corresponding to Ser-34, Asp-306 and His-309 in the Aeromonas hydrophila lipid acyl transferase enzyme shown in FIG. 6 (SEQ ID No. 3) or FIG. 4 (SEQ ID No. 1). As stated above, in the sequence shown in SEQ ID No. 3 or SEQ ID No. 1 the first 18 amino acid residues form a signal sequence. Ser-34, Asp-306 and His-309 of the full length sequence, that is the protein including the signal sequence, equate to Ser-16, Asp-288 and His-291 of the mature part of the protein, i.e. the sequence without the signal sequence. In the pfam00657 consensus sequence, as given in FIG. 5 (SEQ ID No. 2) the active site residues correspond to Ser-7, Asp-345 and His-348.

In one embodiment, the lipid acyl transferase enzyme for use in any one of the methods and/or uses of the present invention is a lipid acyl transferase that may be characterised using the following criteria:
the enzyme possesses acyl transferase activity which may be defined as ester transfer activity whereby the acyl part of an original ester bond of a first lipid acyl donor is transferred to an acyl acceptor to form a new ester; and
the enzyme comprises at least Gly-32, Asp-33, Ser-34, Asp-134 and His-309 or comprises glycine, aspartic acid, serine, aspartic acid and histidine residues at positions corresponding to Gly-32, Asp-33, Ser-34, Asp-306 and His-309, respectively, in the Aeromonas hydrophila lipid acyltransferase enzyme shown in SEQ ID No. 3 or SEQ ID No. 1.

Suitably, the lipid acyltransferase enzyme for use in any one of the methods and/or uses of the present invention may be encoded by one of the following nucleotide sequences:

(a) the nucleotide sequence shown as SEQ ID No. 36;
(b) the nucleotide sequence shown as SEQ ID No. 39;
(c) the nucleotide sequence shown as SEQ ID No. 42;
(d) the nucleotide sequence shown as SEQ ID No. 44;
(e) the nucleotide sequence shown as SEQ ID No. 46;
(t) the nucleotide sequence shown as SEQ ID No. 48;
(g) the nucleotide sequence shown as SEQ ID No. 49;
(h) the nucleotide sequence shown as SEQ ID No. 50;
(i) the nucleotide sequence shown as SEQ ID No. 51;
(j) the nucleotide sequence shown as SEQ ID No. 52;
(k) the nucleotide sequence shown as SEQ ID No. 53;
(l) the nucleotide sequence shown as SEQ ID No. 54;
(m) the nucleotide sequence shown as SEQ ID No. 55;
(n) the nucleotide sequence shown as SEQ ID No. 56;
(o) the nucleotide sequence shown as SEQ ID No. 57;
(p) the nucleotide sequence shown as SEQ ID No. 58;
(q) the nucleotide sequence shown as SEQ ID No. 59;
(r) the nucleotide sequence shown as SEQ ID No. 60;
(s) the nucleotide sequence shown as SEQ ID No. 61;
(t) the nucleotide sequence shown as SEQ ID No. 62;
(u) the nucleotide sequence shown as SEQ ID No. 63;
(v) or
a nucleotide sequence which has 70% or more, preferably 75% or more, identity with any one of the sequences shown as SEQ ID No. 36, SEQ ID No. 39, SEQ ID No. 42, SEQ ID No. 44, SEQ ID No. 46, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 56, SEQ ID No. 57, SEQ ID No. 58, SEQ ID No. 59, SEQ ID No. 60, SEQ ID No. 61, SEQ ID No. 62 or SEQ ID No. 63.

Suitably the nucleotide sequence may have 80% or more, preferably 85% or more, more preferably 90% or more and even more preferably 95% or more identity with any one of the sequences shown as SEQ ID No. 36, SEQ ID No. 39, SEQ ID No. 42, SEQ ID No. 44, SEQ ID No. 46, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 56, SEQ ID No. 57, SEQ ID No. 58, SEQ ID No. 59, SEQ ID No. 60, SEQ ID No. 61, SEQ ID No. 62 or SEQ ID No. 63.

In one embodiment, the nucleotide sequence encoding a lipid acyltransferase enzyme for use any one of the methods and uses of the present invention is a nucleotide sequence which has 70% or more, preferably 75% or more, identity with any one of the sequences shown as: SEQ ID No. 49, SEQ ID No. 50. SEQ ID No. 51, SEQ ID No. 62, and SEQ ID No. 63. Suitably the nucleotide sequence may have 80% or more, preferably 85% or more, more preferably 90% or more and even more preferably 95% or more identity with any one of the sequences shown as: SEQ ID No. 49, SEQ ID No. 50, SEQ ID No. 51, SEQ ID No. 62, and SEQ ID No. 63.

In one embodiment, the nucleotide sequence encoding a lipid acyltransferase enzyme for use in any one of the methods and uses of the present invention is a nucleotide sequence which has 70% or more, 75% or more, 80% or more, preferably 85% or more, more preferably 90% or more and even more preferably 95% or more identity the sequence shown as SEQ ID No. 49.

Suitably, the lipid acyl transferase enzyme for use in any one of the methods and/or uses of the present invention may be a lipid acyltransferase that comprises one or more of the following amino acid sequences:

(i) the amino acid sequence shown as SEQ ID No. 3
(ii) the amino acid sequence shown as SEQ ID No. 4
(iii) the amino acid sequence shown as SEQ ID No. 5
(iv) the amino acid sequence shown as SEQ ID No. 6
(v) the amino acid sequence shown as SEQ ID No. 7
(vi) the amino acid sequence shown as SEQ ID No. 8
(vii) the amino acid sequence shown as SEQ ID No. 9
(viii) the amino acid sequence shown as SEQ ID No. 10
(ix) the amino acid sequence shown as SEQ ID No. 11
(x) the amino acid sequence shown as SEQ ID No. 12
(xi) the amino acid sequence shown as SEQ ID No. 13
(xii) the amino acid sequence shown as SEQ ID No. 14
(xiii) the amino acid sequence shown as SEQ ID No. 1
(xiv) the amino acid sequence shown as SEQ ID No. 15
(xv) the amino acid sequence shown as SEQ ID No. 16
(xvi) the amino acid sequence shown as SEQ ID No. 17
(xvii) the amino acid sequence shown as SEQ ID No. 18
(xviii) the amino acid sequence shown as SEQ ID No. 19
(xix) the amino acid sequence shown as SEQ ID No. 26
(xx) the amino acid sequence shown as SEQ ID No. 27
(xxi) the amino acid sequence shown as SEQ ID No. 28
(xxii) the amino acid sequence shown as SEQ ID No. 29
(xxiiiv) the amino acid sequence shown as SEQ ID No. 30
(xxiv) the amino acid sequence shown as SEQ ID No. 31
(xxv) the amino acid sequence shown as SEQ ID No. 32
(xxvi) the amino acid sequence shown as SEQ ID No. 33
(xxvii) the amino acid sequence shown as SEQ ID No. 34
(xxviii) the amino acid sequence shown as SEQ ID No. 35
(xxix) the amino acid sequence shown as SEQ ID No. 37
(xxx) the amino acid sequence shown as SEQ ID No. 38
(xxxi) the amino acid sequence shown as SEQ ID No. 40
(xxxii) the amino acid sequence shown as SEQ ID No. 41
(xxxiiiv) the amino acid sequence shown as SEQ ID No. 43
(xxxiv) the amino acid sequence shown as SEQ ID No. 45
(xxxv) the amino acid sequence shown as SEQ ID No. 47
(xxxvi) the amino acid sequence shown as SEQ ID No. 48
(xxxvii) the amino acid sequence shown as SEQ ID No. 68
(xxxviii) the amino acid sequence shown as SEQ ID No. 121
(xxxix) the amino acid sequence shown as SEQ ID No. 122
(xl) the amino acid sequence shown as SEQ ID No. 123 or
an amino acid sequence which has 75%, 80%, 85%, 90%, 95%, 98% or more identity with any one of the sequences shown as SEQ ID No. 1, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 26, SEQ ID No. 27, SEQ ID No. 28, SEQ ID No. 29, SEQ ID No. 30. SEQ ID No. 31, SEQ ID No. 32. SEQ ID No. 33, SEQ ID No. 34, SEQ ID No. 35, SEQ ID No. 37, SEQ ID No. 38, SEQ ID No. 40, SEQ ID No. 41, SEQ ID No. 43. SEQ ID No. 45, SEQ ID No. 47, SEQ ID No. 48, SEQ ID No. 68, SEQ ID No. 121, SEQ ID No. 122 or SEQ ID No. 123.

Suitably, the lipid acyl transferase enzyme for use in any one of the methods and uses of the present invention may be a lipid acyitransferase that comprises either the amino acid sequence shown as SEQ ID No. 3 or as SEQ ID No. 4 or SEQ ID No. 1 or SEQ ID No. 15 or SEQ ID No. 16, or SEQ ID No. 34, SEQ ID No. 35, SEQ ID No. 68, SEQ ID No. 121, SEQ ID No. 122 or SEQ ID No. 123 or comprises an amino acid sequence which has 75% or more, preferably 80% or more, preferably 85% or more, preferably 90% or more, preferably 95% or more, identity with the amino acid sequence shown as SEQ ID No. 3 or the amino acid sequence shown as SEQ ID No. 4 or the amino acid sequence shown as SEQ ID No. 1 or the amino acid sequence shown as SEQ ID No. 15 or the amino acid sequence shown as SEQ ID No. 16 or the amino acid sequence shown as SEQ ID No. 34 or the amino acid sequence shown as SEQ ID No. 35 or the amino acid sequence shown as SEQ ID No. 68 or the amino acid sequence shown as SEQ ID No. 121 or the amino acid sequence shown as SEQ ID No. 122 or the amino acid sequence shown as SEQ ID No. 123.

Suitably the lipid acyl transferase enzyme for use any one of the methods and/or uses of the present invention may be a lipid acyltransferase that comprises an amino acid sequence which has 80% or more, preferably 85% or more, more preferably 90% or more and even more preferably 95% or more identity with any one of the sequences shown as SEQ ID No. 16, SEQ ID No. 68, SEQ ID No. 121, SEQ ID No. 122 or SEQ ID No. 123.

Suitably, the lipid acyl transferase enzyme for use any one of the methods and/or uses of the present invention may be a lipid acyltransferase that comprises one or more of the following amino acid sequences:
(a) an amino acid sequence shown as amino acid residues 1-100 of SEQ ID No. 3 or SEQ ID No. 1;
(b) an amino acid sequence shown as amino acids residues 101-200 of SEQ ID No. 3 or SEQ ID No. 1;
(c) an amino acid sequence shown as amino acid residues 201-300 of SEQ ID No. 3 or SEQ ID No. 1; or
(d) an amino acid sequence which has 75% or more, preferably 85% or more, more preferably 90% or more, even more preferably 95% or more identity to any one of the mino acid sequences defined in (a)-(c) above.

Suitably, the lipid acyl transferase enzyme for use in methods and uses of the present invention may comprise one or more of the following amino acid sequences:
(a) an amino acid sequence shown as amino acid residues 28-39 of SEQ ID No. 3 or SEQ ID No. 1;
(b) an amino acid sequence shown as amino acids residues 77-88 of SEQ ID No. 3 or SEQ ID No. 1;
(c) an amino acid sequence shown as amino acid residues 126-136 of SEQ ID No. 3 or SEQ ID No. 1;
(d) an amino acid sequence shown as amino acid residues 163-175 of SEQ ID No. 3 or SEQ ID No. 1;
(e) an amino acid sequence shown as amino acid residues 304-311 of SEQ ID No. 3 or SEQ ID No. 1; or
(f) an amino acid sequence which has 75% or more, preferably 85% or more, more preferably 90% or more, even more preferably 95% or more identity to any one of the amino acid sequences defined in (a)-(e) above.

In one aspect, the lipid acyl transferase enzyme for use any one of the methods and/or uses of the present invention is a lipid acyltransferase that may be the lipid acyl transferase from Candida parapsilosis as taught in EP 1 275 711. Thus in one aspect the lipid acyl transferase for use in the method and uses of the present invention may be a lipid acyl transferase comprising one of the amino acid sequences taught in SEQ ID No. 17 or SEQ ID No. 18.

Much by preference, the lipid acyl transferase enzyme for use in any one of the methods and uses of the present invention is a lipid acyltransferase that may be a lipid acyl transferase comprising the amino acid sequence shown as SEQ ID No. 16, or an amino acid sequence which has 75% or more, preferably 85% or more, more preferably 90% or more, even more preferably 95% or more, even more preferably 98% or more, or even more preferably 99% or more identity to SEQ ID No. 16. This enzyme could be considered a variant enzyme.

In one aspect, the lipid acyltransferase enzyme for use any one of the methods and/or uses of the present invention is a lipid acyltransferase that may be a lecithin:cholesterol acyltransferase (LCAT) or variant thereof (for example a variant made by molecular evolution)

Suitable LCATs are known in the art and may be obtainable from one or more of the following organisms for example: mammals, rat, mice, chickens, Drosophila melanogaster, plants, including Arabidopsis and Oryza sativa, nematodes, fungi and yeast.

In one embodiment the lipid acyltransferase enzyme for use any one of the methods and/or uses of the present invention is a lipid acyltransferase that may be the lipid acyltransferase obtainable, preferably obtained, from the E. coli strains TOP 10 harbouring pPet12aAhydro and pPet12aASalmo deposited by Danisco A/S of Langebrogade 1, DK-1001 Copenhagen K, Denmark under the Budapest Treaty on the International Recognition of the Deposit of Microorganisms for the purposes of Patent Procedure at the National Collection of Industrial, Marine and Food Bacteria (NCIMB) 23 St. Machar Street, Aberdeen, Scotland, United Kingdom on 22 Dec. 2003 under accession numbers NCIMB 41204 and NCIMB 41205, respectively.

A lipid acyltransferase enzyme for use in any one of the methods and/or uses of the present invention may be a phospholipid glycerol acyl transferase. Phospholipid glycerol acyl transferases include those isolated from Aeromonas spp., preferably Aeromonas hydrophila or A. salmonicida, most preferably A. salmonicida or variants thereof.

Most preferred lipid acyl transferases for use in the present invention are encoded by SEQ ID No.s 1, 3, 4, 15, 16, 34 and 35. It will be recognised by the skilled person that it is preferable that the signal peptides of the acyl transferase has been cleaved during expression of the transferase. The signal peptide of SEQ ID No.s 1, 3, 4, and 15 are amino acids 1-18. Therefore the most preferred regions are amino acids 19-335 for SEQ ID No. 1 and SEQ ID No. 3 (A. hydrophilia) and amino acids 19-336 for SEQ ID No. 4, and SEQ ID No. 15 (A. salmonicida). When used to determine the homology of identity of the amino acid sequences, it is preferred that the alignments as herein described use the mature sequence.

In one embodiment, suitably the lipid acyl transferase for use in the present invention comprises (or consists of) the amino acid sequence shown in SEQ ID No. 16 or comprises (or consists of) an amino acid sequence which has at least 70%, at least 75%, at least 85%, at least 90%, at least 95%, at least 98% identity to SEQ ID No. 16.

In one embodiment, suitably the lipid acyl transferase for use in the present invention is encoded by a nucleotide sequence comprising (or consisting of) a nucleotide sequence shown in SEQ ID No. 49 or comprises (or consists of) a nucleotide sequence which has at least 70%, at least 75%, at least 85%, at least 90%, at least 95%, at least 98% identity to SEQ ID No. 49.

Therefore the most preferred regions for determining homology (identity) are amino acids 19-335 for SEQ ID No. 1 and 3 (A. hydrophilia) and amino acids 19-336 for SEQ ID No.s 4, 15 (A. salmonicida). SEQ ID No.s 34 and 35 are mature protein sequences of a lipid acyl transferase from A. hydrophilia and A. salmonicida respectively which may or may not undergo further post-translational modification.

A lipid acyltransferase enzyme for use any one of the methods and uses of the present invention may be a lipid acyltransferase that may also be isolated from Thermobifida, preferably T. fusca, most preferably that encoded by SEQ ID No. 28.

In a preferable embodiment the enzyme for use in accordance with the present invention and/or in the methods of the present invention may comprise any one of the following amino acid sequences and/or be encoded by the following nucleotide sequences:

a) a nucleic acid which encodes a polypeptide exhibiting lipid acyltransferase activity and is at least 70% identical (preferably at least 80%, more preferably at least 90% identical) with the polypeptide sequence shown in SEQ ID No. 16 or with the polypeptide shown in SEQ ID no. 68 or with the polypeptide shown in SEQ ID no. 121 or with the polypeptide shown in SEQ ID no. 122 or with the polypeptide shown in SEQ ID no. 123;
b) a (isolated) polypeptide comprising (or consisting of) an amino acid sequence as shown in SEQ ID No. 16 or SEQ ID No. 68 or an amino acid sequence which is at least 70% identical (preferably at least 80% identical, more preferably at least 90% identical) with SEQ ID No. 16, SEQ ID No. 68, SEQ ID No. 121, SEQ ID No. 122 or SEQ ID No. 123;
c) a nucleic acid encoding a lipid acyltransferase, which nucleic acid comprises (or consists of) a nucleotide sequence shown as SEQ ID No. 49 or a nucleotide sequence which is at least 70% identical (preferably at least 80%, more preferably at least 90% identical) with the nucleotide sequence shown as SEQ ID No. 49;
d) a nucleic acid which hybridises under medium or high stringency conditions to a nucleic acid probe comprising the nucleotide sequence shown as SEQ ID No. 49 and encodes for a polypeptide exhibiting lipid acyltransferase activity;
e) a nucleic acid which is a fragment of the nucleic acid sequences specified in a), c) or d); or
f) a polypeptide which is a fragment of the polypeptide specified in b).

A lipid acyltransferase enzyme for use any one of the methods and uses of the present invention may be a lipid acyttransferase that may also be isolated from Streptomyces, preferable S. avermitis, most preferably that encoded by SEQ ID No. 32. Other possible enzymes for use in the present invention from Streptomyces include those encoded by SEQ ID No.s 5, 6, 9, 10, 11, 12, 13, 14, 31, and 33.

An enzyme for use in the invention may also be isolated from Corynebacterium, preferably C. efficiens, most preferably that encoded by SEQ ID No. 29.

Suitably, the lipid acyltransferase enzyme for use any one of the methods and/or uses of the present invention may be a lipid acyitransferase that comprises any one of the amino acid sequences shown as SEQ ID Nos. 37, 38, 40, 41, 43, 45, or 47 or an amino acid sequence which has at least 70%, 75%, 80%, 85%, 90%, 95%. 96%, 97% or 98% identity therewith, or may be encoded by any one of the nucleotide sequences shown as SEQ ID Nos. 36, 39, 42, 44, 46, or 48 or a nucleotide sequence which has at least 70%. 75%. 80%, 85%. 90%, 95%, 96%, 97% or 98% identity therewith.

In one embodiment, the nucleotide sequence encoding a lipid acyltransferase enzyme for use any one of the methods and/or uses of the present invention is selected from the group consisting of:
a) a nucleic acid comprising a nucleotide sequence shown in SEQ ID No. 36;
b) a nucleic acid which is related to the nucleotide sequence of SEQ ID No. 36 by the degeneration of the genetic code; and
c) a nucleic acid comprising a nucleotide sequence which has at least 70% identity with the nucleotide sequence shown in SEQ ID No. 36.

In one embodiment, the lipid acyltransferase enzyme for use any one of the methods and/or uses of the present invention is a lipid acyltransferase that comprises an amino acid sequence as shown in SEQ ID No. 37 or an amino acid sequence which has at least 60% identity thereto.

In a further embodiment the lipid acyltransferase enzyme for use any one of the methods and/or uses of the present invention may be a lipid acyltransferase comprising any one of the amino acid sequences shown as SEQ ID No. 37, 38, 40, 41, 43, 45 or 47 or an amino acid sequence which has at least 70%, 75%, 80%, 85%, 90%, 95%. 96%, 97% or 98% identity therewith, or may be encoded by any one of the nucleotide sequences shown as SEQ ID No. 39, 42, 44, 46 or 48 or a nucleotide sequence which has at least 70%, 75%, 80%, 85%, 90%, 95%, 96%, 97% or 98% identity therewith.

In a further embodiment the lipid acyltransferase enzyme for use any one of the methods and/or uses of the present invention may be a lipid acyltransferase comprising any one of amino sequences shown as SEQ ID No. 38, 40, 41, 45 or 47 or an amino acid sequence which has at least 70%, 75%, 80%, 85%, 90%, 95%, 96%, 97% or 98% identity therewith for the uses described herein.

In a further embodiment the lipid acyltransferase for use in any one of the methods and/or uses of the present invention may be a lipid acytransferase comprising any one of amino sequences shown as SEQ ID No. 38, 40, or 47 or an amino acid sequence which has at least 70%, 75%, 80%, 85%, 90%, 95%, 96%, 97% or 98% identity therewith for the uses described herein.

More preferably in one embodiment the lipid acyltransferase for use in any one of the methods and/or uses of the present invention may be a lipid acyltransferase comprising the amino acid sequence shown as SEQ ID No. 47 or an amino acid sequence which has at least 70%, 75%, 80%, 85%, 90%, 95%, 96%, 97% or 98% identity therewith.

In another embodiment the lipid acyltransferase for use in any one of the methods and uses of the present invention may be a lipid acyltransferase comprising the amino acid sequence shown as SEQ ID No. 43 or 44 or an amino acid sequence which has at least 80%, 85%, 90%, 95%, 96%, 97% or 98% identity therewith.

In another embodiment the lipid acyltransferase for use in any one of the methods and uses of the present invention may be a lipid acyltransferase comprising the amino acid sequence shown as SEQ ID No. 41 or an amino acid sequence which has at least 70%, 75%, 80%, 85%. 90%, 95%, 96%, 97% or 98% identity therewith.

In one embodiment the lipid acyltransferase for use in any one of the methods and uses of the present invention may be encoded by a nucleic acid selected from the group consisting of:
a) a nucleic acid comprising a nucleotide sequence shown in SEQ ID No. 36;
b) a nucleic acid which is related to the nucleotide sequence of SEQ ID No. 36 by the degeneration of the genetic code; and
c) a nucleic acid comprising a nucleotide sequence which has at least 70% identity with the nucleotide sequence shown in SEQ ID No. 36.

In one embodiment the lipid acyltransferase according to the present invention may be a lipid acyltransferase obtainable, preferably obtained, from the Streptomyces strains L130 or L131 deposited by Danisco A/S of Langebrogade 1, DK-1001 Copenhagen K, Denmark under the Budapest Treaty on the International Recognition of the Deposit of Microorganisms for the purposes of Patent Procedure at the National Collection of Industrial, Marine and Food Bacteria (NCIMB) 23 St. Machar Street, Aberdeen, Scotland, United Kingdom on 23 Jun. 2004 under accession numbers NCIMB 41226 and NCIMB 41227, respectively.

Suitable nucleotide sequences encoding a lipid acyitransferase for use in any one of the methods and/or uses of the present invention may encode a polynucleotide encoding a lipid acyltransferase (SEQ ID No. 16); or may encode an amino acid sequence of a lipid acyltransferase (SEQ ID No. 16).

A suitable lipid acyltransferases for use in any one of the methods and/or uses of the present invention may be an amino acid sequence which may be identified by alignment to the L131 (SEQ ID No. 37) sequence using Align X, the Clustal W pairwise alignment algorithm of VectorNTI using default settings.

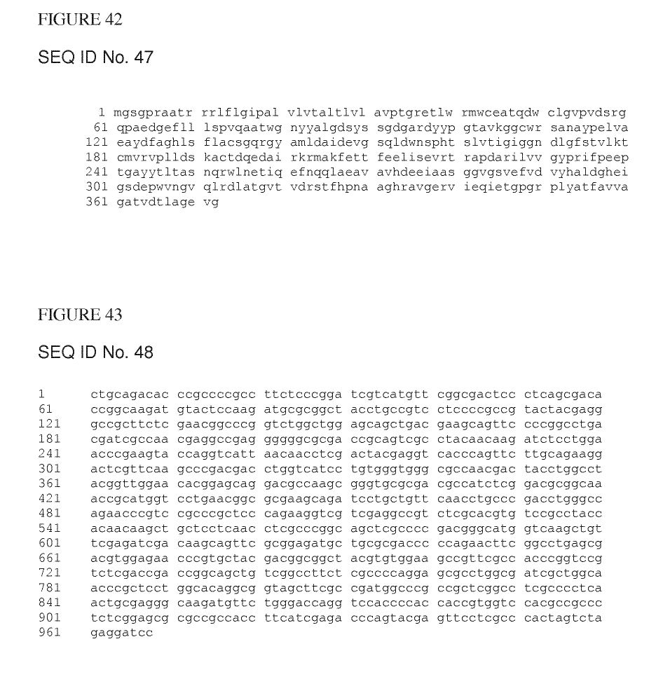

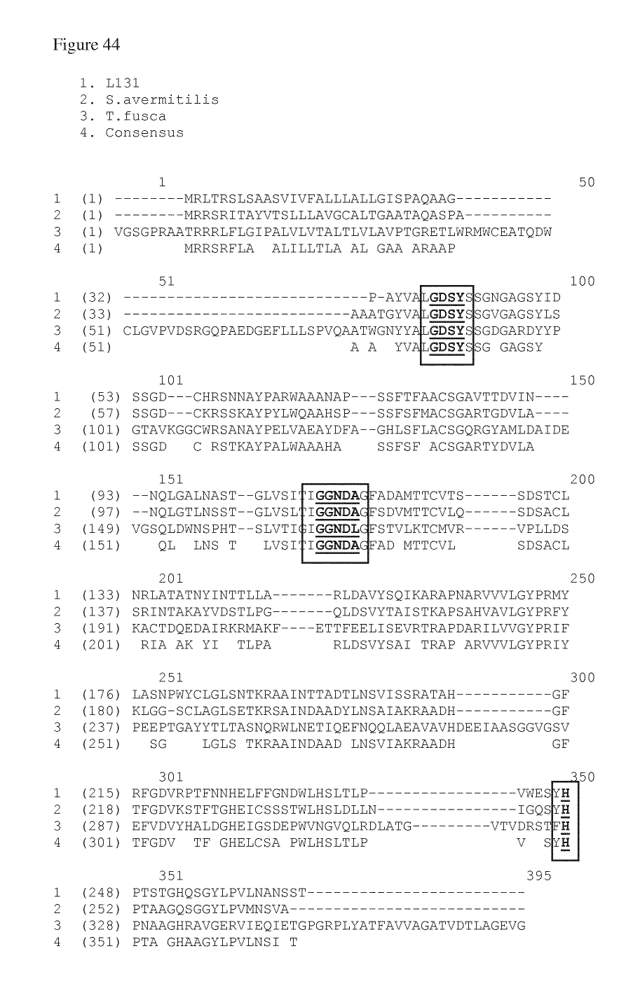

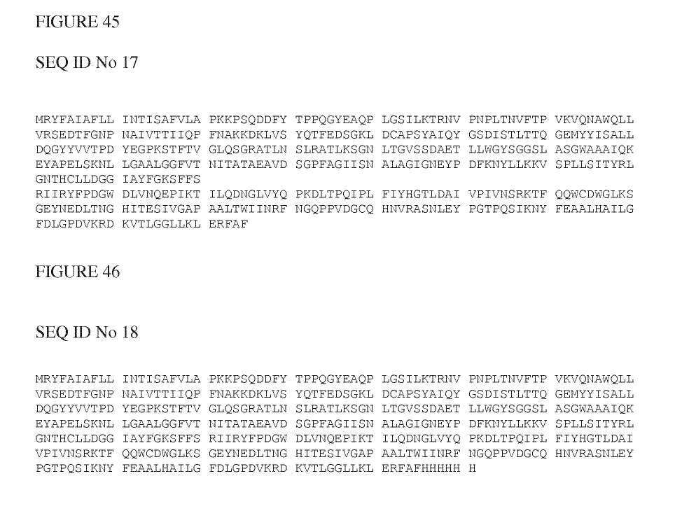

An alignment of the L131 and homologues from S. avermitilis and T. fusca illustrates that the conservation of the GDSx motif (GDSY in L131 and S. avermitilis and T. fusca), the GANDY box, which is either GGNDA or GGNDL, and the HPT block (considered to be the conserved catalytic histidine). These three conserved blocks are highlighted in FIG. 42.

When aligned to either the pfam Pfam00657 consensus sequence (as described in WO 2004/064987) and/or the L131 sequence herein disclosed (SEQ ID No 37) it is possible to identify three conserved regions, the GDSx block, the GANDY block and the HTP block (see WO 2004/064987 for further details).

When aligned to either the pfam Pfam00657 consensus sequence (as described in WO 2004/064987) and/or the L131 sequence herein disclosed (SEQ ID No 37)
i) The lipid acyltransferase for use in any one of the methods and uses of the present invention may be a lipid acyltransferase that has a GDSx motif, more preferably a GDSx motif selected from GDSL or GDSY motif.
and/or
ii) The lipid acyltransferase for use in any one of the methods and uses of the present invention may be a lipid acyttransferase that has a GANDY block, more preferably a GANDY block comprising amino GGNDx, more preferably GGNDA or GGNDL.
and/or
iii) The lipid acyltransferase for use in any one of the methods and uses of the present invention may be a lipid acyltransferase that has preferably an HTP block.
and preferably
iv) the lipid acyltransferase for use in any one of the methods and uses of the present invention may be a lipid acyltransferase that has preferably a GDSx or GDSY motif, and a GANDY block comprising amino GGNDx, preferably GGNDA or GGNDL, and a HTP block (conserved histidine).

The lipid acyltransferase as used herein may be referred to as a glycerophospholipid cholesterol acyitransferase. In other words the lipid acyltransferase for use in the present invention preferably has the ability to “de-esterify” phospholipids and at the same time esterify cholesterol with the free fatty acid from the hydrolyzation this is effectively a tranferase reaction (i.e. a transesterification reaction. The degree of “de-esterification” can be described as the ratio of phosphatidylcholine (PC) and/or phosphatidylethanolamine (PE) converted into lyso-PC or lyso-PE respectively.

By the enzymatic de-esterification of PC into lyso-PC, the ratio between the hydrophilic part of the phospholipid molecule (polar head group) and the hydrophobic part (fatty acid chains) is altered. By removing one fatty acid (saturated and/or unsaturated fatty acids) the hydrophobic part is reduced, thus making the entire molecule more hydrophilic. Furthermore the sterical molecule conformation may be changed, which may influence phase structures (e.g. micellation) formed by the molecules in dispersion, as well as interactions with other molecules like e.g. milk proteins.

Lyso-lecithin products are known to possess improved emulsifying properties. With a high degree of interesterification and/or transesterification it is possible to obtain smaller mean oil droplet sizes in a comparative emulsification test.

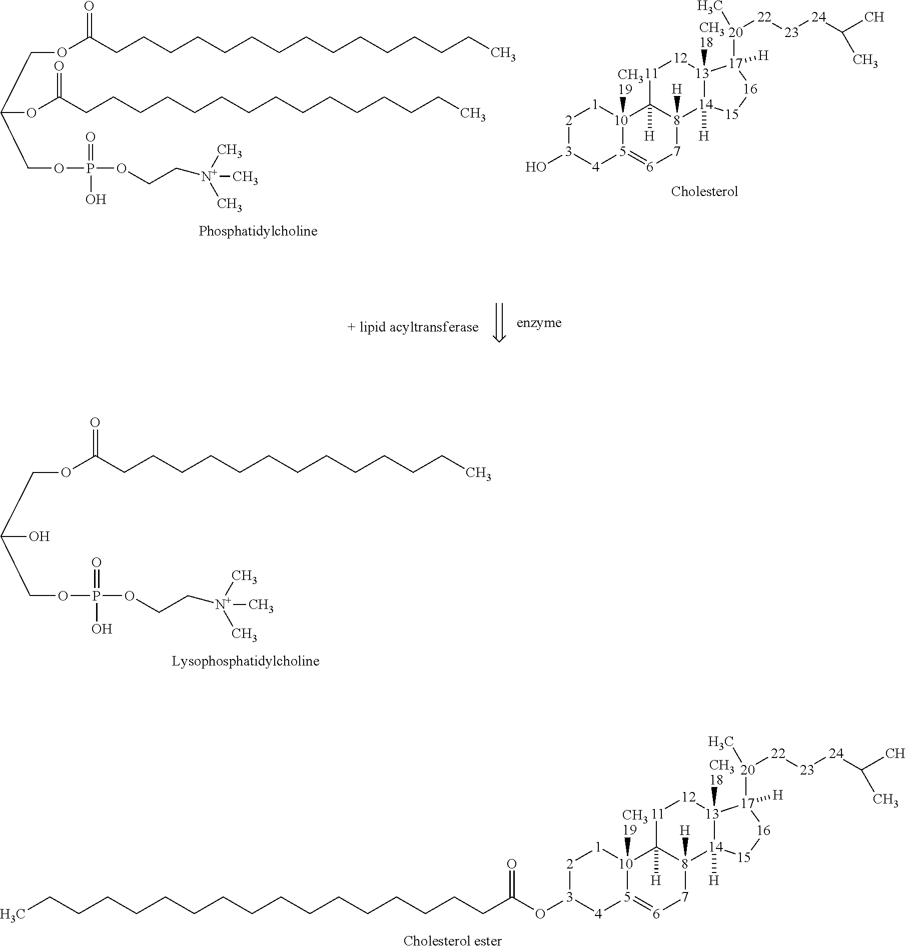

The function of lipid acyltransferase is that cholesterol and phospholipids will be changed into cholesterol-esters and lyso-phospholipids.

The lipid acyltransferase may be a variant lipid acyltransferase. Production of such variants is taught in WO2011/045629 which is incorporated herein by reference.

In one embodiment the lipid acyitransferase enzyme may be the enzyme sold as LysoMax Oil ™ (available from Danisco A/S).

In one embodiment the lipid acyitransferase enzyme may be an enzyme sold as FoodPro® Cleanline (available from Danisco A/S)

In a preferred embodiment the lipid acyltransferase for use in accordance with the present invention may be SEQ ID No. 16 as taught in WO2011/045629.

Enzymes which function as lipid acyltransferases in accordance with the present invention can be routinely identified using the assay taught herein below:

### Determination of Transferase Activity—Assay (Cholesterol:Phospholipid)” (Tru)

Substrate: 50 mg Cholesterol (Sigma C8503) and 450 mg Soya phosphatidylcholine (PC), Avanti #441601 is dissolved in chloroform, and chloroform is evaporated at 40° C. under vacuum.

300 mg PC:cholesterol 9:1 is dispersed at 40° C. in 15 ml 50 mM HEPES buffer pH 7.

### Enzymation:

1 ml substrate is added in a glass with lid at 40° C. μl enzyme solution is added and incubated during agitation for 10 minutes at 40° C.

The enzyme added should esterify 0.6-3% of the cholesterol in the assay.

Also a blank with 25 μl water instead of enzyme solution is analysed.

After 10 minutes 5 ml Hexan:Isopropanol 3:2 is added.

Lipids are extracted into the organic phase and the upper organic phase is isolated.

The amount of cholesterol ester is analysed by HPTLC or HPLC using Cholesteryl stearate (Sigma C3549) standard for calibration.

Transferase activity is calculated as the amount of cholesterol ester formation per minute under assay conditions.

One Transferase Unit (TrU) is defined as μmol cholesterol ester produced per minute at 40° C. and pH 7 in accordance with the transferase assay given above.

Preferably, the lipid acyltransferase used in the method and uses of the present invention will have a specific transferase unit (TrU) per mg enzyme when using the assay above of at least TrU/mg enzyme protein.

Suitably the lipid acyltransferase for use in the present invention may be dosed in amount of 0.05 to 50 TrU per g oil, suitably in an amount of 0.5 to 5 TrU per g oil.

More preferably the enzymes suitable for use in the methods and/or uses of the present invention have lipid acyl-transferase activity as defined by the protocol below:

### Protocol for the Determination of % Acyltransferase Activity:

An edible oil to which a lipid acyltransferase according to the present invention has been added may be extracted following the enzymatic reaction with CHCl₃:CH₃OH 2:1 and the organic phase containing the lipid material is isolated and analysed by GLC and HPLC according to the procedure detailed hereinbelow. From the GLC and HPLC analyses the amount of free fatty acids and one or more of sterol/stanol esters; are determined. A control edible oil to which no enzyme according to the present invention has been added, is analysed in the same way.

### Calculation:

From the results of the GLC and HPLC analyses the increase in free fatty acids and sterol/stanol esters can be calculated:

Δ % fatty acid=% Fatty acid (enzyme)−% fatty acid (control);

Mv fatty acid=average molecular weight of the fatty acids;

A=Δ % sterol ester/Mv sterol ester (where Δ % sterol ester=% sterol/stanol ester (enzyme)−% sterol/stanol ester (control) and Mv sterol ester=average molecular weight of the sterol/stanol esters);

The transferase activity is calculated as a percentage of the total enzymatic activity:

$$\% transferase activity = \frac{A \times 100}{A + Δ \% fatty acid / ( Mv fatty acid )}$$

If the free fatty acids are increased in the edible oil they are preferably not increased substantially, i.e. to a significant degree. By this we mean, that the increase in free fatty acid does not adversely affect the quality of the edible oil.

The edible oil used for the acyltransferase activity assay is preferably the soya bean oil supplemented with plant sterol (1%) and phosphatidylcholine (2%) oil using the method:
Plant sterol and phosphatidylcholine were dissolved in soya bean oil by heating to 95° C. during agitation. The oil was then cooled to 40° C. and the enzymes were added. Water was added to a total concentration of 5% of the oil phase. The sample was maintained at 40° C. with magnetic stirring and samples were taken out after 4 and 20 hours and analysed by TLC.

For the assay the enzyme dosage used is preferably 0.2 TIPU-K/g oil, more preferably 0.08 TIPU-K/g oil, preferably 0.01 TIPU-K/g oil. The level of phospholipid present in the oil and/or the % conversion of sterol is preferably determined after 0.5, 1, 2, 4 and 20 hours, more preferably after 20 hours.

When the enzyme used in the palm fruit extract (e.g. pressed palm fruit extract) is a lipid acyttransferase enzyme preferably the incubation time is effective to ensure that there is at least 5% transferase activity, preferably at least 10% transferase activity, preferably at least 15%, 20%, 25% 26%, 28%, 30%, 40% 50%, 60% or 75% transferase activity.

The % transferase activity (i.e. the transferase activity as a percentage of the total enzymatic activity) may be determined by the protocol taught above.

An enzyme is a lipid acyl transferase if it results in at least 5% (preferably at least 10% transferase activity, preferably at least 20%, preferably at least 25%, preferable at least 30% transferase activity) in the assay taught above.

### Phospholipase Enzymes

Suitably the enzyme for use in the present invention is a phospholipase A1 and/or phospholipase A2 and/or phospholipase B and/or phospholipase D enzyme. Phospholipase A1 and A2 enzymes hydrolyse phospholipids (e.g. lecithin) into fatty acids and other lipophilic substances. Phospholipase A1 enzymes hydrolyse or cleave the SN-1 acyl chain. Phospholipase A2 enzymes hydrolyse or cleave the SN-2 acyl chain. Phospholipase A2 enzymes act on the intact phospholipid (e.g. lecithin molecule) and hydrolyses the fatty acid esterified to the second carbon atom. The resulting products are lysolecithin and a fatty acid. The phospholipase cleavage sites are shown in FIG. 2. Notably phospholipase A1 is shown as PLA1 and phospholipase A2 is shown as PLA2. PLC and PLD are phospholipase C and phospholipase D respectively.

PLC is not an enzyme for use in the present invention.

In one embodiment the enzyme for use in the present invention may be a PLD enzyme.

In an alternative embodiment the enzyme for use in the present invention may not be a phospholipase D.

An enzyme that displays both PLA1 and PLA2 activity is called a phospholipase B. In one embodiment the enzyme for use in the present invention may be a phospholipase B.

In another embodiment the enzyme according to the present invention is not a phospholipase B enzyme.

Suitably the enzyme for use in the present invention is a phospholipase A1 enzyme. Phospholipase A1 enzymes can be classified as E.C. 3.1.1.32.

Suitably the enzyme for use in the present invention is a phospholipase A2 enzyme. Phospholipase A2 enzymes can be classified as E.C. 3.1.1.4.

Suitably the enzyme for use in the present invention is a phospholipase B enzyme. Phospholipase B enzymes can be classified as E.C. 3.1.1.5.

Suitably, the enzyme for use in the present invention is a phospholipase D enzyme. Phospholipase D enzymes can be classified as E.C. 3.1.4.4.

In one embodiment the enzyme may be selected from the group consisting of Lecitase Ultra (a phospholipase A1 enzyme) available from Novozymes A/S, Lipopan F (a phospholipase A1) available from Novozymes A/S, Nagase PLA2 (available from Nagase Chemtex Corporation, 1-1-17, Shinmachi, Nishi-ku, Osaka 550-8668, Japan), GL541 PLA2 (which has the amino acid sequence (shown as SEQ ID No. 124 in FIG. 76) APADKPQVLASFTQTSASSQNAWLAANRNQSAWAAYEFDWSTDLCTQAPDNPFGFPFNTA CARHDFGYRNYKAAGSFDANKSRIDSAFYEDMKRVCTGYTGEKNTACNSTAWTYYQAVKI FG). Maxapal™ or Cakezyme (both pancreatic PLA2 expressed in Apspergillus as taught in WO2010/124975) available from DSM, Panamore™ (a PLA1) available from DSM, Lipomod 699™ (a pork pancreatic phospholipase 2 [PLA2]) available from Biocatalysts, Rohalase™ PL-XTRA (a PLA1) available from AB enzymes, or YieldMax™ (PLA1) available from Chr Hansen and Novozymes.

In one embodiment the enzyme for use in the present invention may (optionally in combination with any of those above) may be selected from the group consisting of: G-ZYME™ G999 (available from Danisco A/S) (PLB) or Rohalase®F (available from AB Enzymes GmbH, Feldbergstrasse 78, 64293 Darmstadt, Germany).

The phospholipase D according to the present invention may be any publically available Phospholipase D. In one embodiment the phospholipase D may be Phospholipase D from Streptomyces sp. (available from Sigma, P4912).

Phospholipase A1 or A2 enzymes can be selected using the Assay taught below:

### Determination of Phospholipase Activity (TIPU-K Assay) Assay:

Substrate:

1.75% Avanti Lecithin (#441601), 6.3% Triton X-100 (Sigma #T9284, peroxide free), 50 mM HEPES, pH 7.0, and 5.0 mM CaCl₂.

### Assay Procedure:

34 μl substrate was added to a cuvette, using a KoneLab automatic analyzer. At time T=0 min, 4 μl enzyme solution was added. Also a blank with water instead of enzyme was analyzed. The sample was mixed and incubated at 30° C. for 10 minutes.

The free fatty acid content of a sample is analyzed by using the NEFA-HR kit from WAKO GmbH. Enzyme activity TIPU pH 7 is calculated as micromole fatty acid produced per minute under assay conditions.

Phospholipase B enzymes can be selected using the Assay taught below:

### Determination of Phospholipase Activity (TIPU-K Assay) Assay:

### Substrate:

1.25% Lyso-phosphatidylcholine Avanti (#845875P), 6.3% Triton X-100 (Sigma #T9284, peroxide free), 50 mM HEPES, pH 7.0, and 5.0 mM CaCl₂.

### Assay Procedure:

34 μl substrate was added to a cuvette, using a KoneLab automatic analyzer. At time T=0 min, 4 μl enzyme solution was added. Also a blank with water instead of enzyme was analyzed. The sample was mixed and incubated at 30° C. for 10 minutes.

The free fatty acid content of a sample is analyzed by using the NEFA-HR kit from WAKO GmbH. Enzyme activity TIPU pH 7 is calculated as micromole fatty acid produced per minute under assay conditions.

### Enzyme Combinations

It may also be beneficial to combine the use of lipid acyltransferase with a phospholipase, such as a phospholipase A1 and/or a phospholipase A2 and/or a phospholipase B and/or a phospholipase D.

In a preferred embodiment a lipid acyttransferase may be combined with a phospholipase A1 and/or a phospholipase A2.

The combined use may be performed sequentially or concurrently, e.g. the lipid acyl transferase treatment may occur prior to or during the further enzyme treatment.

Alternatively, the further enzyme treatment may occur prior to or during the lipid acyl transferase treatment.

In the case of sequential enzyme treatments, in some embodiments it may be advantageous to remove the first enzyme used, e.g. by heat deactivation or by use of an immobilised enzyme, prior to treatment with the second (and/or third etc.) enzyme.

### Dosage

The one or more enzyme(s) for use in the methods and/or uses of the present invention may be dosed at pre-determined amounts when treating the palm fruit or a portion thereof or palm fruit extract.

In one embodiment the one or more enzyme(s) may be dosed at 0.5 to 500 mg/kg palm fruit or extract.

In another embodiment the one or more enzyme(s) may be dosed at 1 to 200 mg/kg palm fruit or extract.

In still another embodiment the one or more enzyme(s) may be dosed at 1 to 100 mg/kg palm fruit or extract. In still other embodiments the one or more enzyme(s) may be dosed at 1 to 50 mg/kg palm fruit or extract.

In a yet further embodiment the one or more enzyme(s) may be dosed at 1 to 10 mg/kg palm fruit or extract.

### Host Cell

The host organism can be a prokaryotic or a eukaryotic organism.

In one embodiment of the present invention the lipid acyl transferase according to the present invention in expressed in a host cell, for example a bacterial cells, such as a Bacillus spp, for example a Bacillus licheniformis host cell.

Alternative host cells may be fungi, yeasts or plants for example.

It has been found that the use of a Bacillus licheniformis host cell results in increased expression of a lipid acyltransferase when compared with other organisms, such as Bacillus subtilis.

In one embodiment preferably the lipid acyltransferase is expressed in Bacillus licheniformis as taught in WO2008/090395 or WO2011/061657, which references are incorporated herein by reference.

A lipid acyltransferase from Aeromonas salmonicida has been inserted into a number of conventional expression vectors, designed to be optimal for the expression in Bacillus subtilis, Hansenula polymorpha, Schizosaccharomyces pombe and Aspergillus tubigensis, respectively.

Only very low levels were, however, detected in Hansenula polymorpha, Schizosaccharomyces pombe and Aspergillus tubigensis. The expression levels were below 1 μg/ml, and it was not possible to select cells which yielded enough protein to initiate a commercial production (results not shown). In contrast, Bacillus licheniformis was able to produce protein levels, which are attractive for an economically feasible production.

In particular, it has been found that expression in B. licheniformis is approximately 100-times greater than expression in B. subtilis under the control of aprE promoter or is approximately 100-times greater than expression in S. lividans under the control of an A4 promoter and fused to cellulose (results not shown herein).

The host cell may be any Bacillus cell other than B. subtilis. Preferably, said Bacillus host cell being from one of the following species: Bacillus licheniformis; B. alkalophilus; B. amyloliquefaciens; B. circulans; B. clausii; B. coagulans; B. firmus; B. lautus; B. lentus; B. megaterium; B. pumilus or B. stearothermophilus.

The term “host cell”—in relation to the present invention includes any cell that comprises either a nucleotide sequence encoding a lipid acyltransferase or a phospholipase as defined herein or an expression vector as defined herein and which is used in the recombinant production of a lipid acyltransferase or phospholipase having the specific properties as defined herein.

Suitably, the host cell may be a protease deficient or protease minus strain and/or an α-amylase deficient or α-amylase minus strain.

The term “heterologous” as used herein means a sequence derived from a separate genetic source or species. A heterologous sequence is a non-host sequence, a modified sequence, a sequence from a different host cell strain, or a homologous sequence from a different chromosomal location of the host cell.

A “homologous” sequence is a sequence that is found in the same genetic source or species i.e. it is naturally occurring in the relevant species of host cell.

The term “recombinant lipid acyltransferase” or “recombinant phospholipase” as used herein means that the lipid acyltransferase or phospholipase has been produced by means of genetic recombination. For instance, the nucleotide sequence encoding the lipid acyltransferase or phospholipase has been inserted into a cloning vector, resulting in a B. licheniformis cell characterised by the presence of the heterologous lipid acyltransferase or phospholipase.

### Regulatory Sequences

In some applications, a lipid acyltransferase or phospholipase sequence for use in the methods and/or uses of the present invention may be obtained by operably linking a nucleotide sequence encoding same to a regulatory sequence which is capable of providing for the expression of the nucleotide sequence, such as by the chosen host cell (such as a B. licheniformis cell).

By way of example, a vector comprising the nucleotide sequence of the present invention operably linked to such a regulatory sequence, i.e. the vector is an expression vector, may be used.

The term “operably linked” refers to a juxtaposition wherein the components described are in a relationship permitting them to function in their intended manner. A regulatory sequence “operably linked” to a coding sequence is ligated in such a way that expression of the coding sequence is achieved under conditions compatible with the control sequences.

The term “regulatory sequences” includes promoters and enhancers and other expression regulation signals.

The term “promoter” is used in the normal sense of the art, e.g. an RNA polymerase binding site.

Enhanced expression of the nucleotide sequence encoding the enzyme having the specific properties as defined herein may also be achieved by the selection of regulatory regions, e.g. promoter, secretion leader and terminator regions that are not regulatory regions for the nucleotide sequence encoding the enzyme in nature.

Suitably, the nucleotide sequence of the present invention may be operably linked to at least a promoter.

Suitably, the nucleotide sequence encoding a lipid acyltransferase or phospholipase may be operably linked to at a nucleotide sequence encoding a terminator sequence. Examples of suitable terminator sequences for use in any one of the vectors, host cells, methods and/or uses of the present invention include: an α-amylase terminator sequence (for instance, CGGGACTTACCGAAAGAAACCATCAATGATGGTTTCTTTTTTGTTCATAAA—SEQ ID No. 64), an alkaline protease terminator sequence (for instance, CAAGACTAAAGACCGTTCGCCCGTTTTTGCAATAAGCGGGCGAATCTTACATAAAAATA—SEQ ID No. 65), a glutamic-acid specific terminator sequence (for instance, ACGGCCGTTAGATGTGACAGCCCGTTCCAAAAGGAAGCGGGCTGTCTTCGTGTATTATTGT—SEQ ID No. 66), a levanase terminator sequence (for instance, TCTTTTAAAGGAAAGGCTGGAATGCCCGGCATTCCAGCCACATGATCATCGTTT—SEQ ID No. 67) and a subtilisin E terminator sequence (for instance, GCTGACAAATAAAAGAAGCAGGTATGGAGGAACCTGCTTCTTTTTACTATTATTG—SEQ ID No. 119). Suitably, the nucleotide sequence encoding a lipid acyltransferase or phospholipase may be operably linked to an α-amylase terminator, such as a B. licheniformis α-amylase terminator.

### Promoter

The promoter sequence to be used in accordance with the present invention may be heterologous or homologous to the sequence encoding a lipid acyltransferase or phospholipase.

The promoter sequence may be any promoter sequence capable of directing expression of a lipid acyltransferase or phospholipase in the host cell of choice.

Suitably, the promoter sequence may be homologous to a Bacillus species, for example B. licheniformis. Preferably, the promoter sequence is homologous to the host cell of choice.

Suitably the promoter sequence may be homologous to the host cell. “Homologous to the host cell” means originating within the host organism: i.e. a promoter sequence which is found naturally in the host organism.

Suitably, the promoter sequence may be selected from the group consisting of a nucleotide sequence encoding: an α-amylase promoter, a protease promoter, a subtilisin promoter, a glutamic acid-specific protease promoter and a levansucrase promoter.

Suitably the promoter sequence may be a nucleotide sequence encoding: the LAT (e.g. the alpha-amylase promoter from B. licheniformis, also known as AmyL), AprL (e.g. subtilisin Carlsberg promoter), EndoGluC (e.g. the glutamic-acid specific promoter from B. licheniformis). AmyQ (e.g. the alpha amylase promoter from B. amyloliquefaciens alpha-amylase promoter) and SacB (e.g. the B. subtilis levansucrase promoter).

Other examples of promoters suitable for directing the transcription of a nucleic acid sequence in the methods of the present invention include: the promoter of the Bacillus lentus alkaline protease gene (aprH); the promoter of the Bacillus subtilis alpha-amylase gene (amyE); the promoter of the Bacillus stearothermophilus maltogenic amylase gene (amyM); the promoter of the Bacillus licheniformis penicillinase gene (penP); the promoters of the Bacillus subtilis xylA and xylB genes; and/or the promoter of the Bacillus thuringiensis subsp. tenebrionis CryIIIA gene.

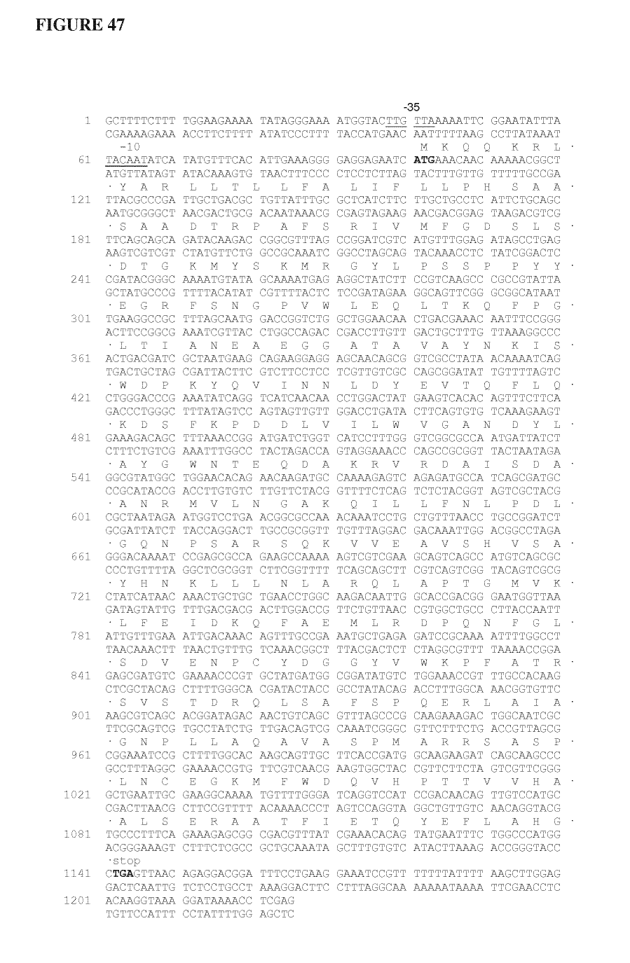

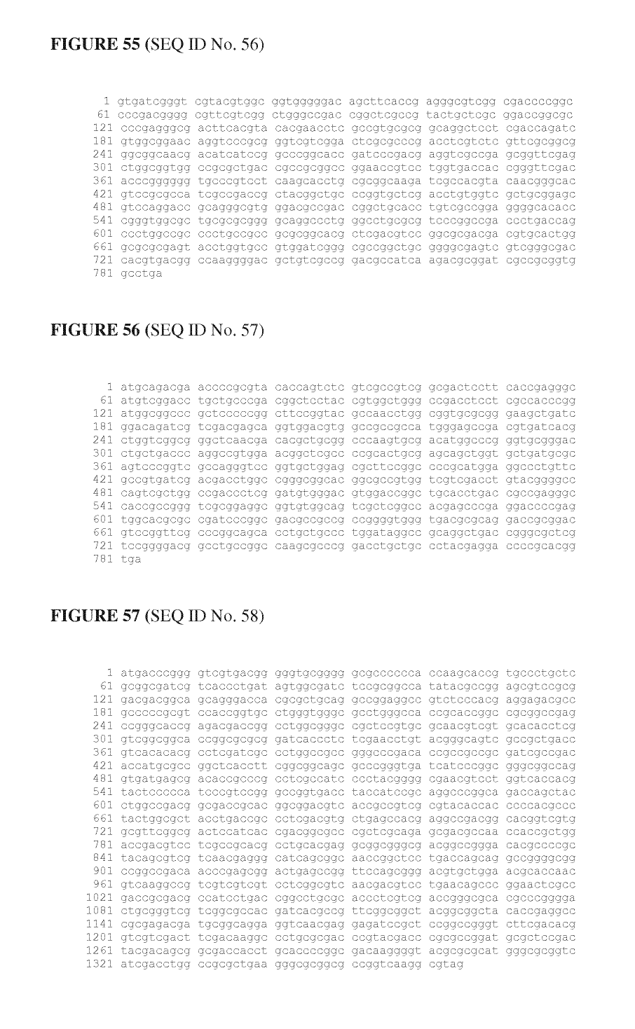

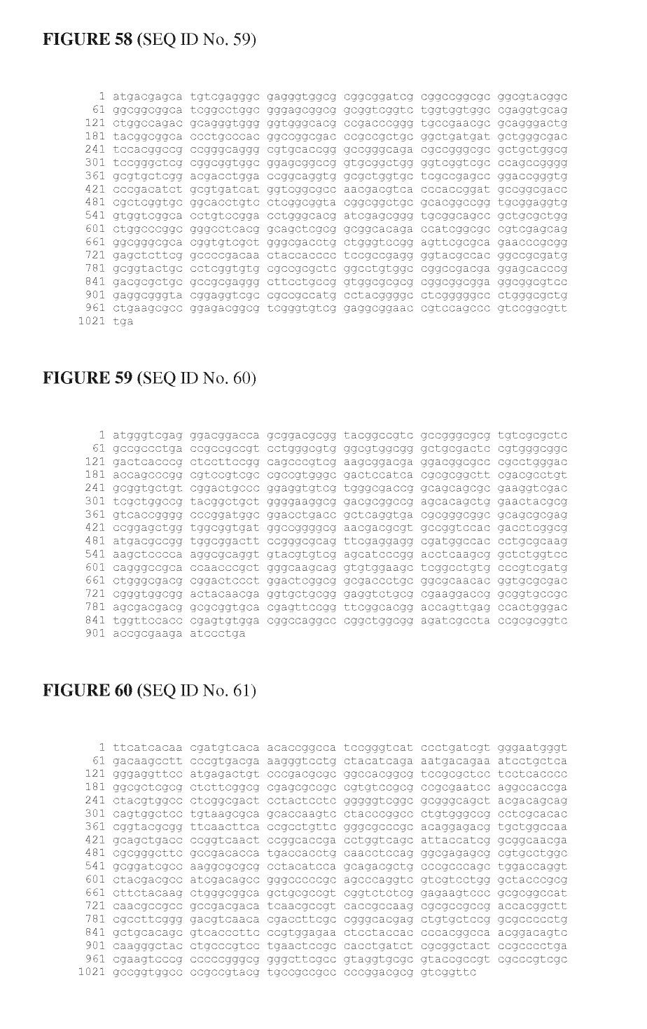

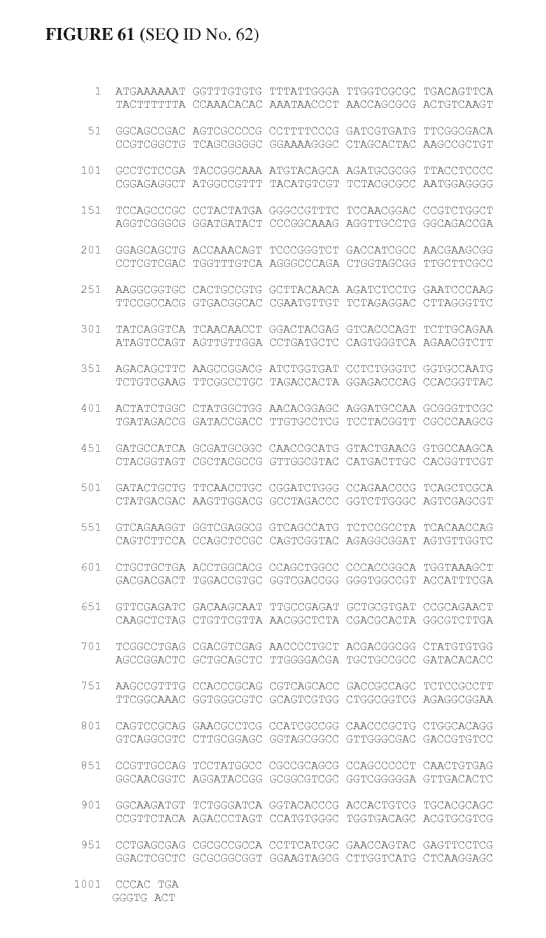

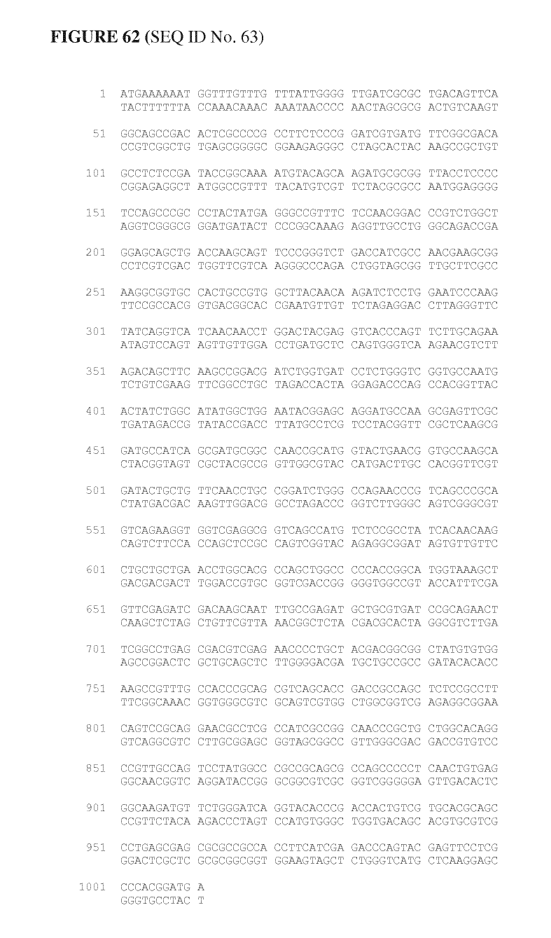

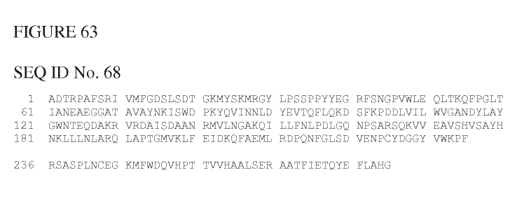

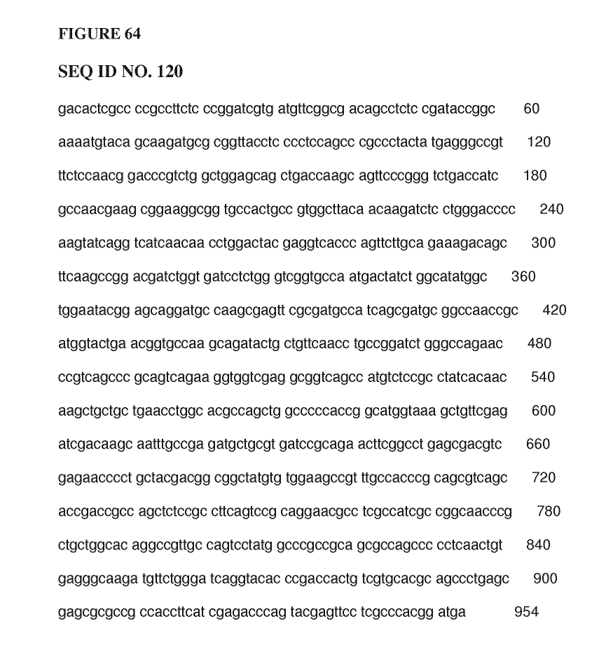

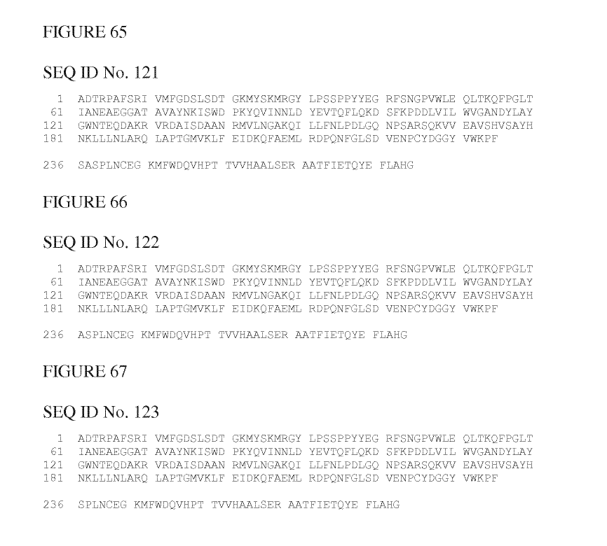

In a preferred embodiment, the promoter sequence is an α-amylase promoter (such as a Bacillus licheniformis α-amylase promoter). Preferably, the promoter sequence comprises the −35 to −10 sequence of the B. licheniformis α-amylase promoter—see FIGS. 53 and 55. The “−35 to −10 sequence” describes the position relative to the transcription start site. Both the “−35” and the “−10” are boxes, i.e. a number of nucleotides, each comprising 6 nucleotides and these boxes are separated by 17 nucleotides. These 17 nucleotides are often referred to as a “spacer”. This is illustrated in FIG. 47, where the −35 and the −10 boxes are underlined. For the avoidance of doubt, where “−35 to −10 sequence” is used herein it refers to a sequence from the start of the −35 box to the end of the −10 box i.e. including both the −35 box, the 17 nucleotide long spacer and the −10 box.

### Signal Peptide

The lipid acyltransferase or phospholipase produced by a host cell by expression of the nucleotide sequence encoding the lipid acyltransferase may be secreted or may be contained intracellularly depending on the sequence and/or the vector used.

A signal sequence may be used to direct secretion of the coding sequences through a particular cell membrane. The signal sequences may be natural or foreign to the lipid acyltransferase coding sequence. For instance, the signal peptide coding sequence may be obtained from an amylase or protease gene from a Bacillus species, preferably from Bacillus licheniformis.

Suitable signal peptide coding sequences may be obtained from one or more of the following genes: maltogenic α-amylase gene, subtilisin gene, beta-lactamase gene, neutral protease gene, prsA gene, and/or acyltransferase gene.

Preferably, the signal peptide is a signal peptide of B. licheniformis α-amylase, Aeromonas acyltransferase (for instance, mkkwfvcllglialtvqa—SEQ ID No. 21), B. subtilis subtilisin (for instance, mrskklwisllfaltliftmafsnmsaqa—SEQ ID No. 22) or B. licheniformis subtilisin (for instance, mmrkksfwfgmitafmlvftmefsdsasa—SEQ ID No. 23). Suitably, the signal peptide may be the signal peptide of B. licheniformis α-amylase.

However, any signal peptide coding sequence capable of directing the expressed lipid acyltransferase into the secretory pathway of a Bacillus host cell (preferably a B. licheniformis host cell) of choice may be used.

In some embodiments of the present invention, a nucleotide sequence encoding a signal peptide may be operably linked to a nucleotide sequence encoding a lipid acyltransferase or phospholipase of choice.

The lipid acyttransferase or phospholipase of choice may be expressed in a host cell as defined herein as a fusion protein.

### Expression Vector

The term “expression vector” means a construct capable of in vivo or in vitro expression.

Preferably, the expression vector is incorporated in the genome of the organism, such as a B. licheniformis host. The term “incorporated” preferably covers stable incorporation into the genome.

The nucleotide sequence encoding a lipid acyltransferase or phospholipase as defined herein may be present in a vector, in which the nucleotide sequence is operably linked to regulatory sequences such that the regulatory sequences are capable of providing the expression of the nucleotide sequence by a suitable host organism (such as B. licheniformis), i.e. the vector is an expression vector.

The vectors of the present invention may be transformed into a suitable host cell as described above to provide for expression of a polypeptide having lipid acyltransferase or phospholipase activity as defined herein.

The choice of vector, e.g. plasmid, cosmid, virus or phage vector, genomic insert, will often depend on the host cell into which it is to be introduced. The present invention may cover other forms of expression vectors which serve equivalent functions and which are, or become, known in the art.

Once transformed into the host cell of choice, the vector may replicate and function independently of the host cell's genome, or may integrate into the genome itself.

The vectors may contain one or more selectable marker genes—such as a gene which confers antibiotic resistance e.g. ampicillin, kanamycin, chloramphenicol or tetracycline resistance. Alternatively, the selection may be accomplished by co-transformation (as described in WO 91/17243).

Vectors may be used in vitro, for example for the production of RNA or used to transfect or transform a host cell.

The vector may further comprise a nucleotide sequence enabling the vector to replicate in the host cell in question. Examples of such sequences are the origins of replication of plasmids pUC19, pACYC177, pUB110, pE194, pAMB1 and pIJ702.

### Post-Transcription and Post-Translational Modifications

Suitably the lipid acyltransferase or phospholipase in accordance with the present invention may be encoded by any one of the nucleotide sequences taught herein.

Depending upon the host cell used post-transcriptional and/or post-translational modifications may be made. It is envisaged that the enzymes (e.g. the lipid acyltransferase or phospholipases) for use in the present methods and/or uses encompasses enzymes (e.g. lipid acyltransferases or phospholipases) which have undergone post-transcriptional and/or post-translational modification.

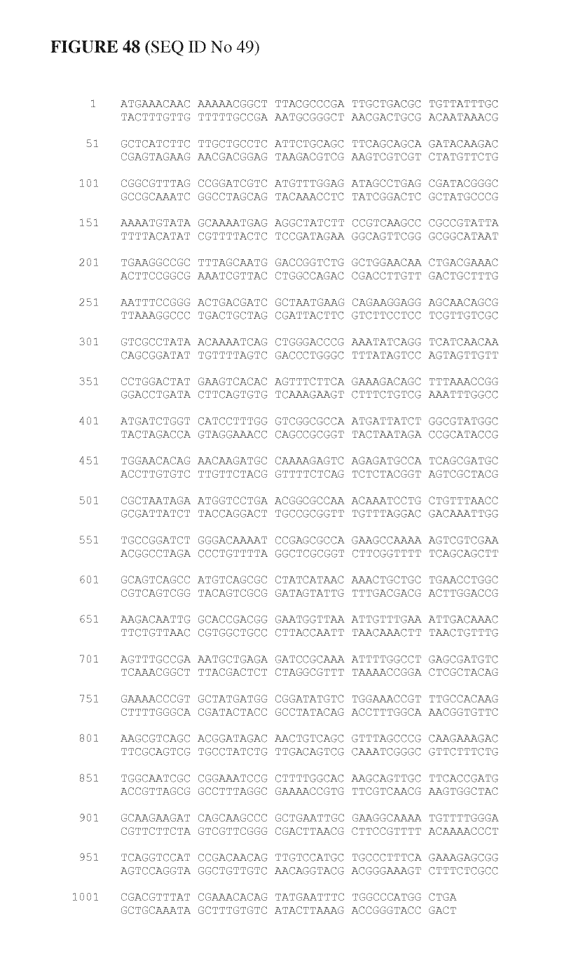

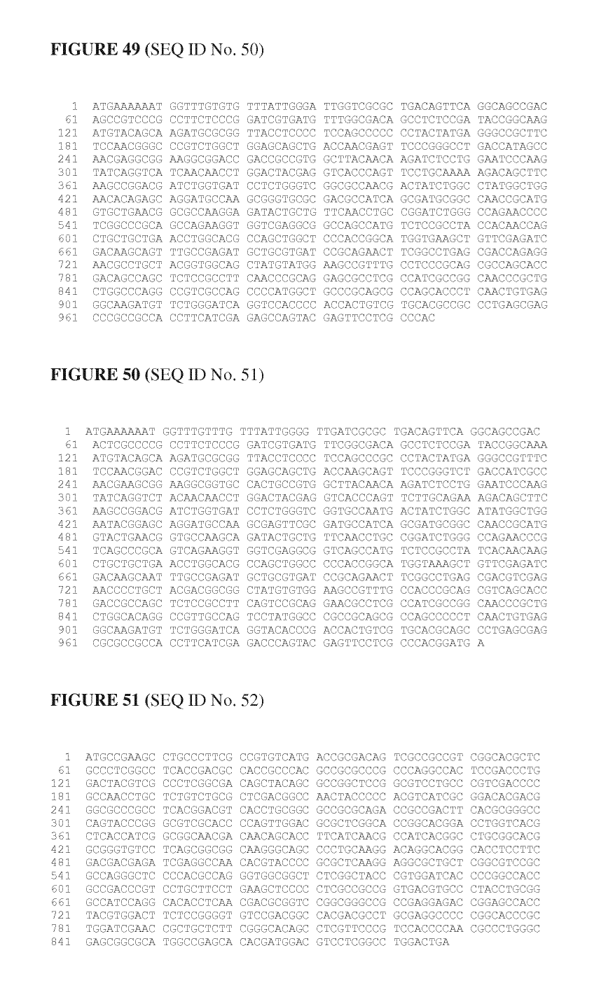

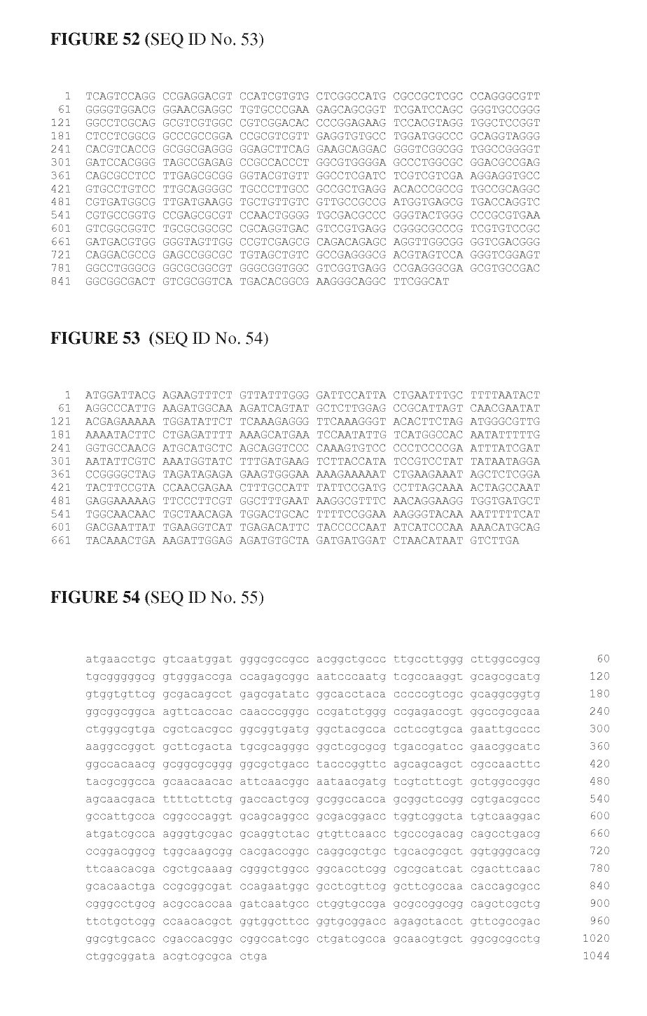

By way of example only, the expression of the nucleotide sequence shown herein as SEQ ID No. 49 (see FIG. 48) in a host cell (such as Bacillus licheniformis for example) results in post-transcriptional and/or post-translational modifications which lead to the amino acid sequence shown herein as SEQ ID No. 68 (see FIG. 73) which encodes a functional lipid acyttransferase.

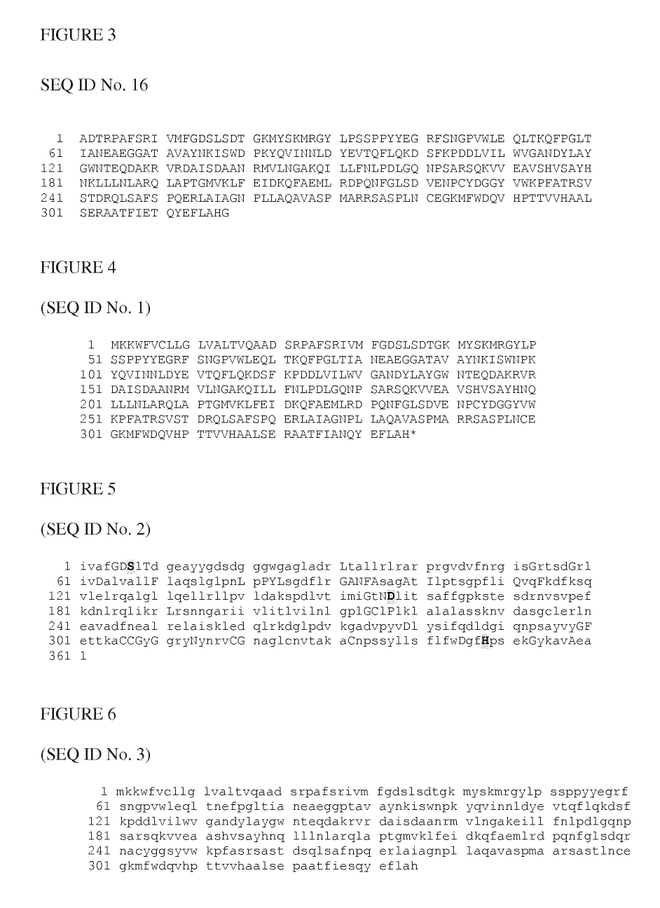

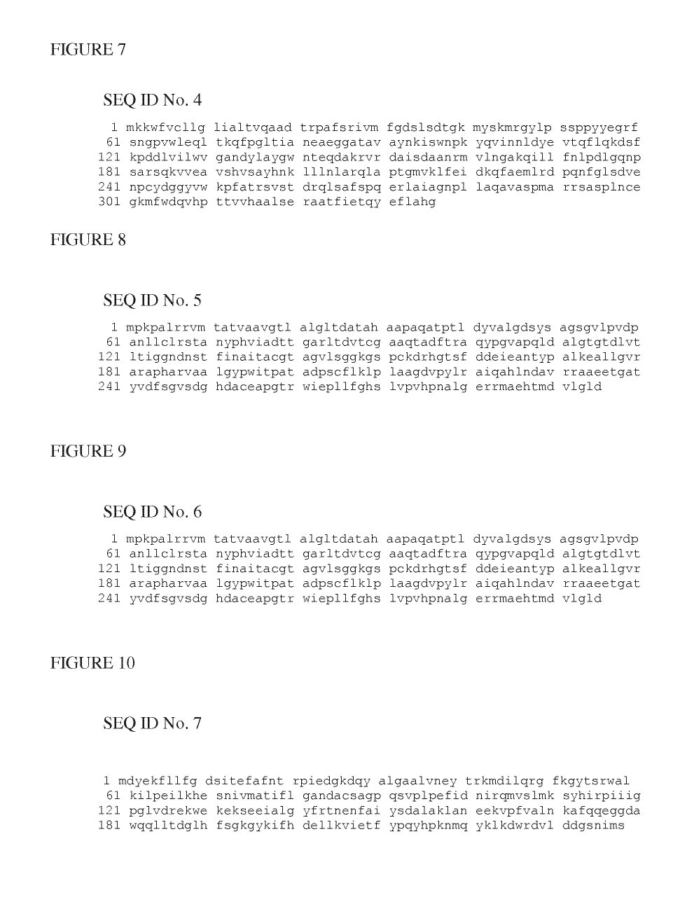

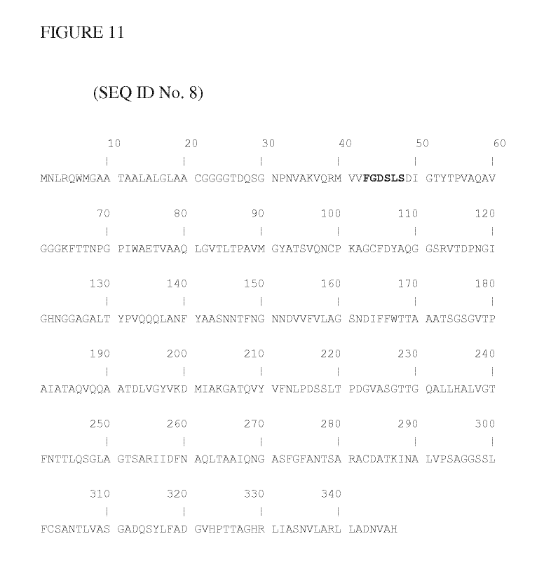

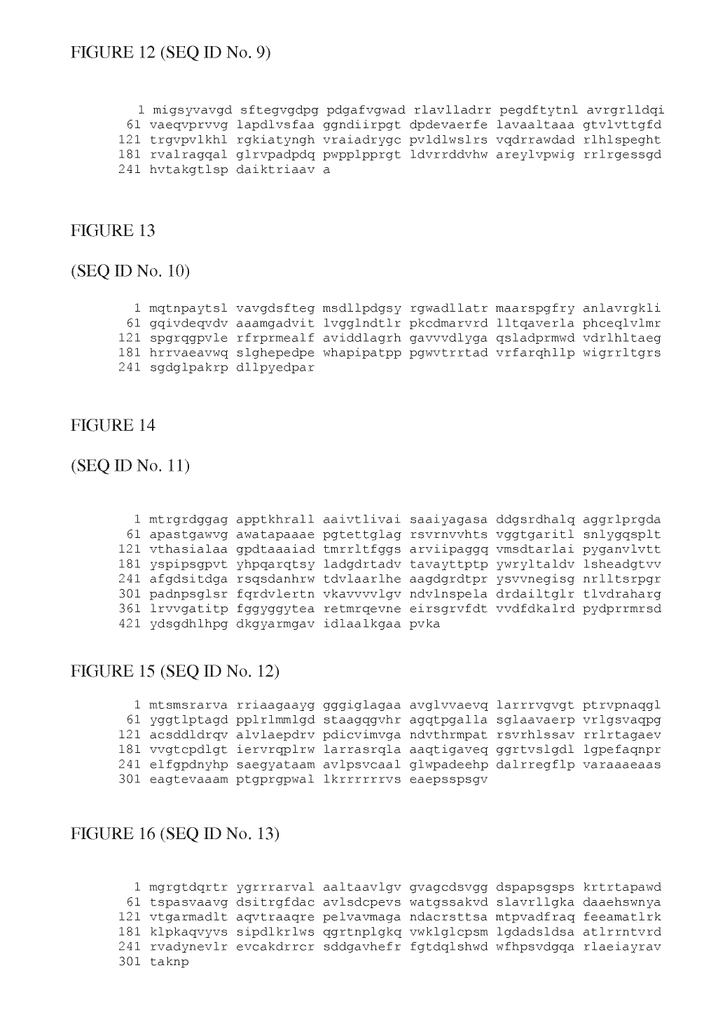

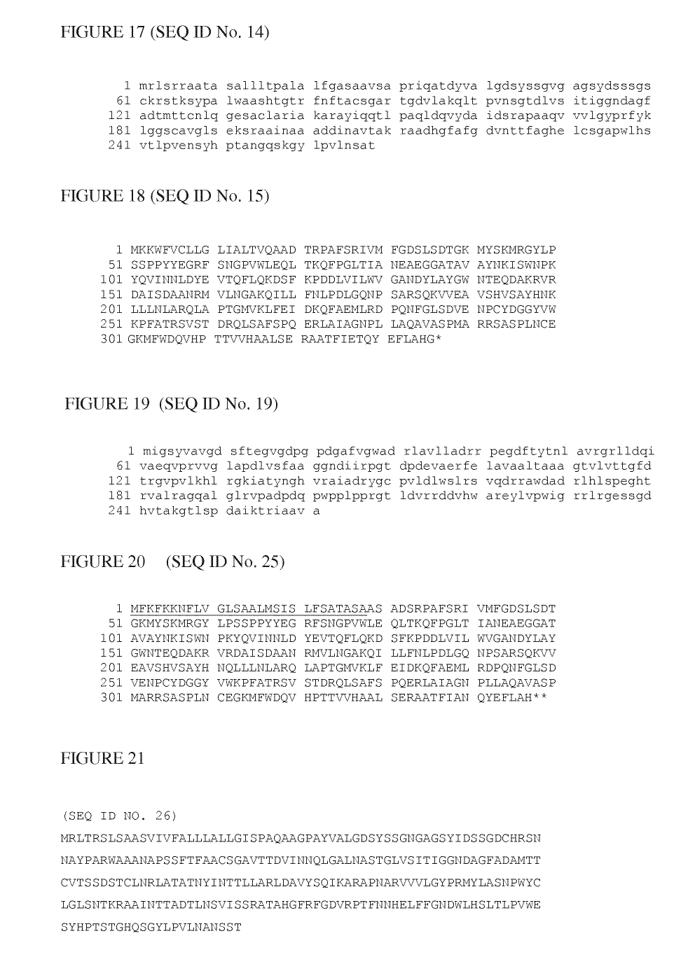

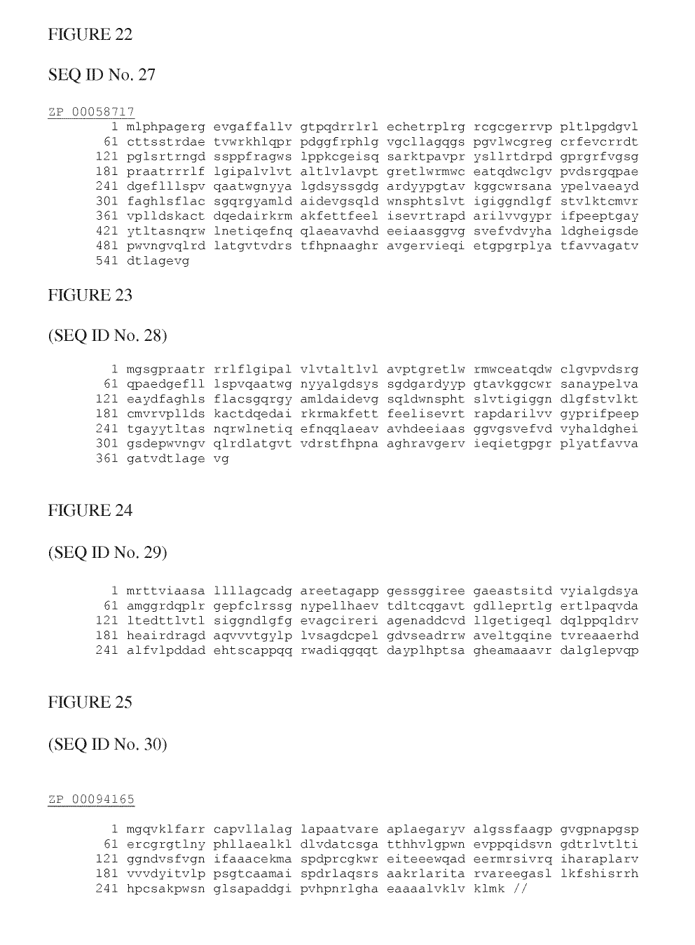

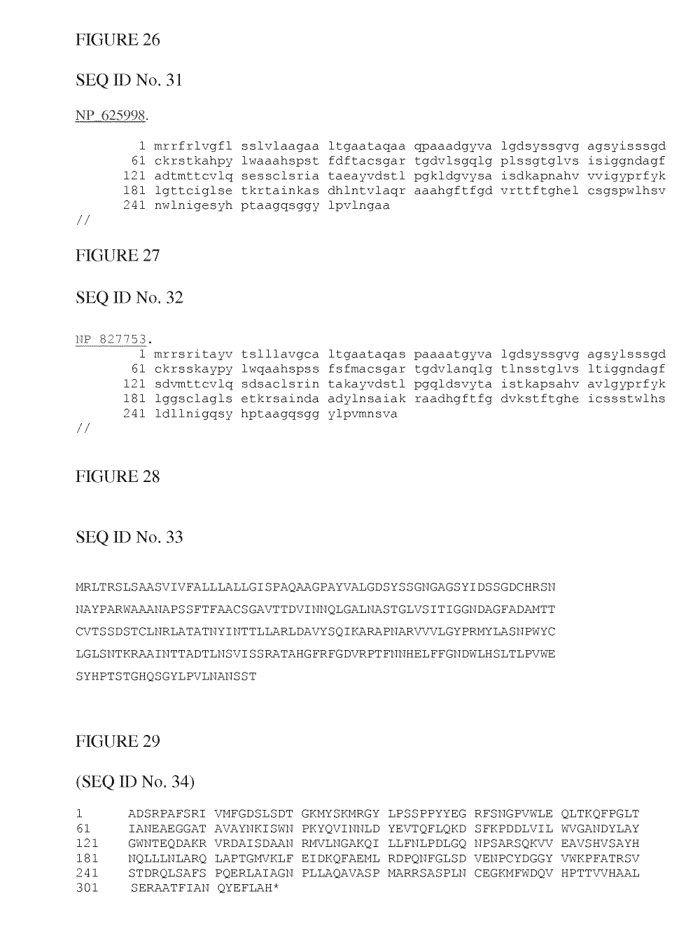

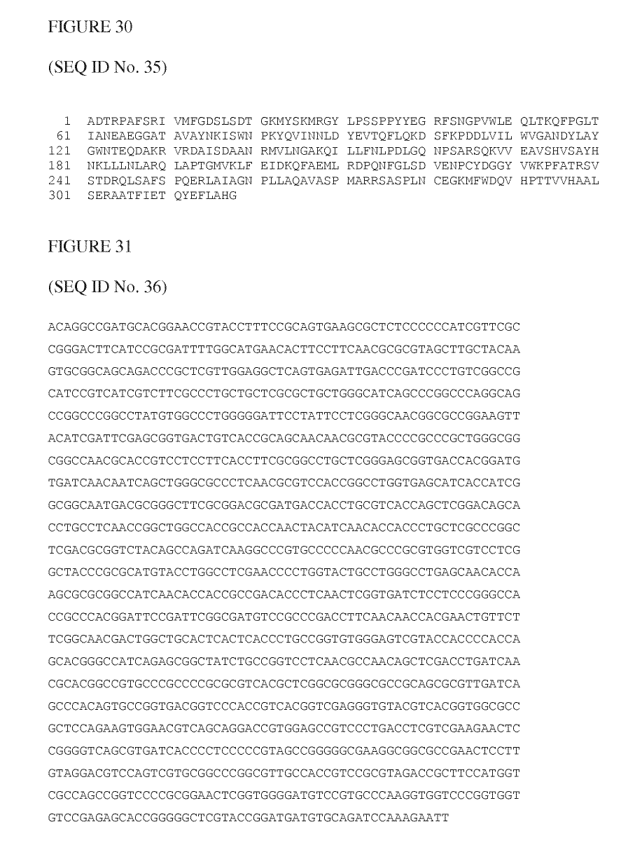

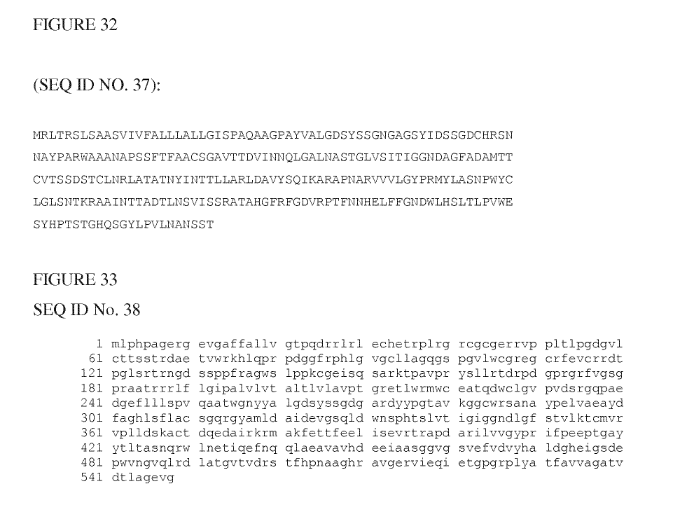

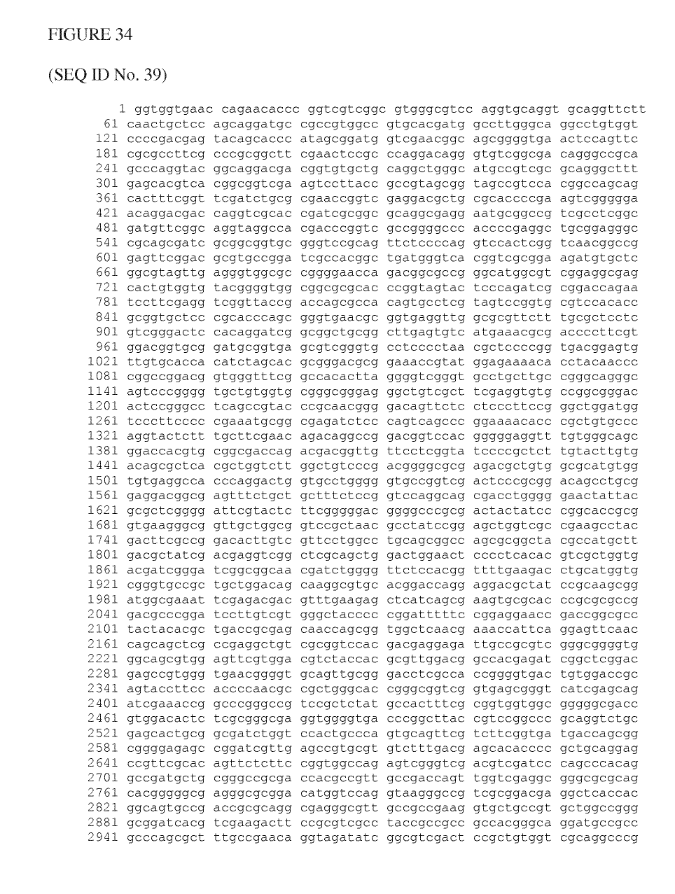

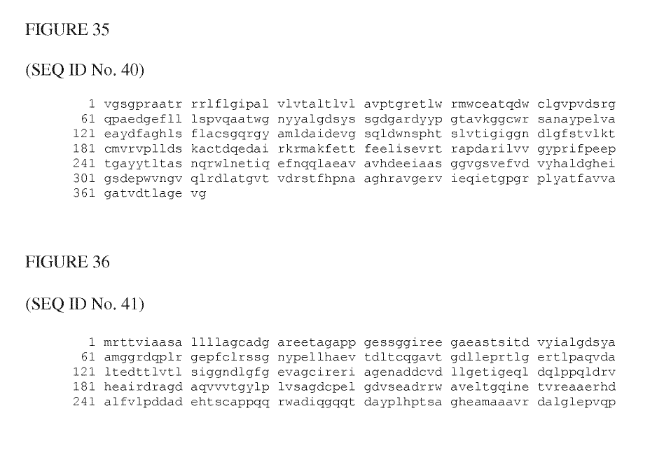

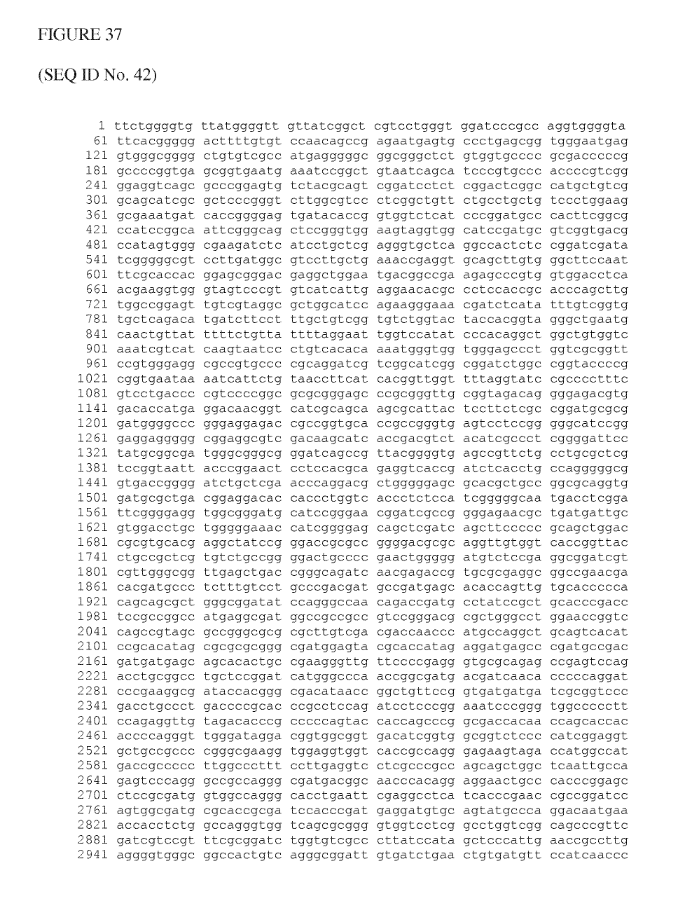

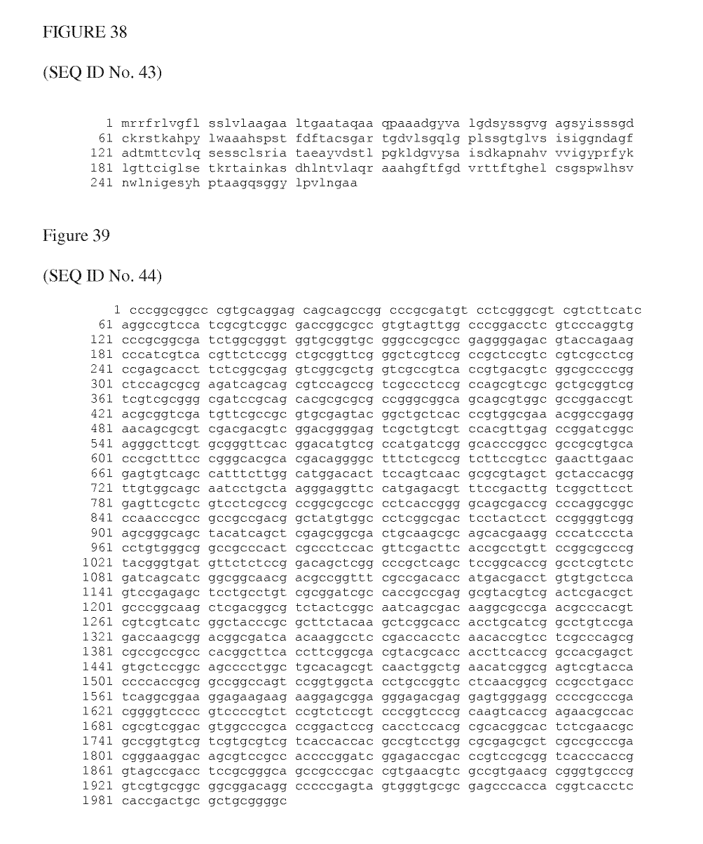

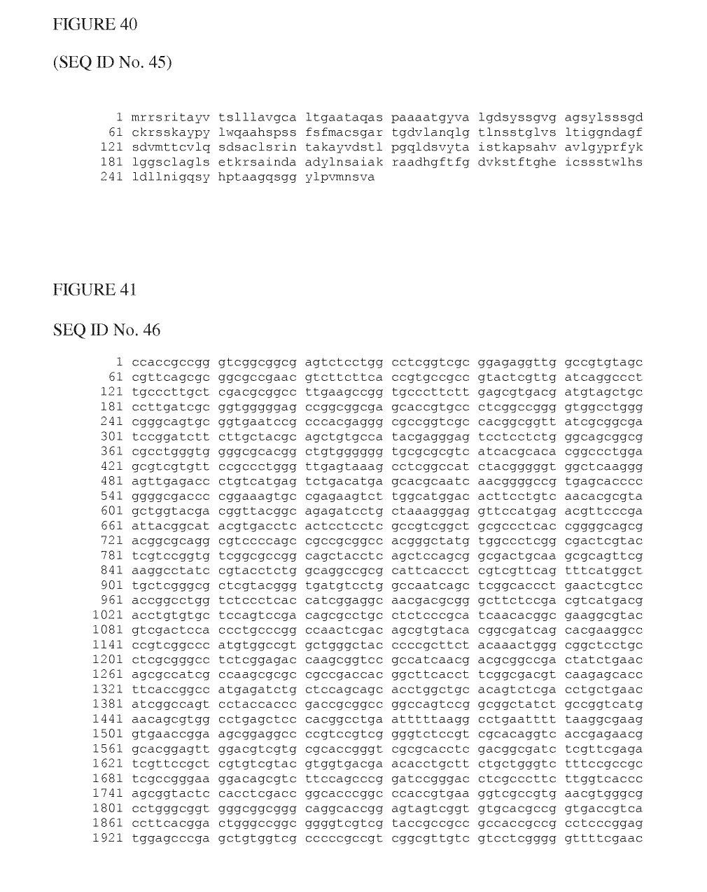

SEQ ID No. 68 is the same as SEQ ID No. 16 (shown herein in FIG. 3) except that SEQ ID No. 68 has undergone post-translational and/or post-transcriptional modification to remove 38 amino acids.

SEQ ID NO. 16 may also be post transcriptionally and/or post translationally modified to remove 39, 40 or 41 amino as shown in SEQ ID NOs. 121, 122 and 123 respectively.

### Isolated

In one aspect, the enzyme (e.g. lipid acyltransferase or phospholipase) is a recovered/isolated enzyme (e.g. lipid acyltransferase or phospholipase). Thus, the enzyme (e.g. lipid acyltransferase or phospholipase) produced may be in an isolated form.

In another aspect, the nucleotide sequence encoding a enzyme (e.g. lipid acyltransferase or phospholipase) for use in the present invention may be in an isolated form.

The term “isolated” means that the sequence or protein is at least substantially free from at least one other component with which the sequence or protein is naturally associated in nature and as found in nature.

### Purified

In one aspect, the enzyme (e.g. lipid acyltransferase or phospholipase) may be in a purified form.

In another aspect, the nucleotide sequence encoding a enzyme (e.g. lipid acyltransferase or phospholipase) for use in the present invention may be in a purified form.

The term “purified” means that the sequence is in a relatively pure state—e.g. at least about 51% pure, or at least about 75%, or at least about 80%, or at least about 90% pure, or at least about 95% pure or at least about 98% pure.

### Cloning a Nucleotide Sequence Encoding a Polypeptide According to the Present Invention

A nucleotide sequence encoding either a polypeptide which has the specific properties as defined herein or a polypeptide which is suitable for modification may be isolated from any cell or organism producing said polypeptide. Various methods are well known within the art for the isolation of nucleotide sequences.

For example, a genomic DNA and/or cDNA library may be constructed using chromosomal DNA or messenger RNA from the organism producing the polypeptide. If the amino acid sequence of the polypeptide is known, labelled oligonucleotide probes may be synthesised and used to identify polypeptide-encoding clones from the genomic library prepared from the organism. Alternatively, a labelled oligonucleotide probe containing sequences homologous to another known polypeptide gene could be used to identify polypeptide-encoding clones. In the latter case, hybridisation and washing conditions of lower stringency are used.

Alternatively, polypeptide-encoding clones could be identified by inserting fragments of genomic DNA into an expression vector, such as a plasmid, transforming enzyme-negative bacteria with the resulting genomic DNA library, and then plating the transformed bacteria onto agar containing an enzyme inhibited by the polypeptide, thereby allowing clones expressing the polypeptide to be identified.

In a yet further alternative, the nucleotide sequence encoding the polypeptide may be prepared synthetically by established standard methods, e.g. the phosphoroamidite method described by Beucage S. L. et al (1981) Tetrahedron Letters 22, p 1859-1869, or the method described by Matthes et al (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et a (Science (1988) 239, pp 487-491).

### Nucleotide Sequences

The present invention also encompasses nucleotide sequences encoding polypeptides having the specific properties as defined herein. The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or antisense strand.

The term “nucleotide sequence” in relation to the present invention includes genomic DNA, cDNA, synthetic DNA, and RNA. Preferably it means DNA, more preferably cDNA for the coding sequence.

In a preferred embodiment, the nucleotide sequence per se encoding a polypeptide having the specific properties as defined herein does not cover the native nucleotide sequence in its natural environment when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. Thus, the polypeptide of the present invention can be expressed by a nucleotide sequence in its native organism but wherein the nucleotide sequence is not under the control of the promoter with which it is naturally associated within that organism.

Preferably the polypeptide is not a native polypeptide. In this regard, the term “native polypeptide” means an entire polypeptide that is in its native environment and when it has been expressed by its native nucleotide sequence.

Typically, the nucleotide sequence encoding polypeptides having the specific properties as defined herein is prepared using recombinant DNA techniques (i.e. recombinant DNA). However, in an alternative embodiment of the invention, the nucleotide sequence could be synthesised, in whole or in part, using chemical methods well known in the art (see Caruthers M H et al (1980) Nuc Acids Res Symp Ser 215-23 and Horn T et al (1980) Nuc Acids Res Symp Ser 225-232).

### Molecular Evolution

Once an enzyme-encoding nucleotide sequence has been isolated, or a putative enzyme-encoding nucleotide sequence has been identified, it may be desirable to modify the selected nucleotide sequence, for example it may be desirable to mutate the sequence in order to prepare an enzyme in accordance with the present invention.

Mutations may be introduced using synthetic oligonucleotides. These oligonucleotides contain nucleotide sequences flanking the desired mutation sites.

A suitable method is disclosed in Morinaga et al (Biotechnology (1984) 2, p 646-649). Another method of introducing mutations into enzyme-encoding nucleotide sequences is described in Nelson and Long (Analytical Biochemistry (1989), 180, p 147-151).

Instead of site directed mutagenesis, such as described above, one can introduce mutations randomly for instance using a commercial kit such as the GeneMorph PCR mutagenesis kit from Stratagene, or the Diversify PCR random mutagenesis kit from Clontech. EP 0 583 265 refers to methods of optimising PCR based mutagenesis, which can also be combined with the use of mutagenic DNA analogues such as those described in EP 0 866 796. Error prone PCR technologies are suitable for the production of variants of lipid acyl transferases with preferred characteristics. WO0206457 refers to molecular evolution of lipases.

A third method to obtain novel sequences is to fragment non-identical nucleotide sequences, either by using any number of restriction enzymes or an enzyme such as Dnase I, and reassembling full nucleotide sequences coding for functional proteins. Alternatively one can use one or multiple non-identical nucleotide sequences and introduce mutations during the reassembly of the full nucleotide sequence. DNA shuffling and family shuffling technologies are suitable for the production of variants of lipid acyl transferases with preferred characteristics. Suitable methods for performing ‘shuffling’ can be found in EP0 752 008, EP1 138 763, EP1 103 606. Shuffling can also be combined with other forms of DNA mutagenesis as described in U.S. Pat. No. 6,180,406 and WO 01/34835.

Thus, it is possible to produce numerous site directed or random mutations into a nucleotide sequence, either in vivo or in vitro, and to subsequently screen for improved functionality of the encoded polypeptide by various means. Using in silico and exo mediated recombination methods (see WO 00/58517, U.S. Pat. No. 6,344,328, U.S. Pat. No. 6,361,974), for example, molecular evolution can be performed where the variant produced retains very low homology to known enzymes or proteins. Such variants thereby obtained may have significant structural analogy to known transferase enzymes, but have very low amino acid sequence homology.

As a non-limiting example, In addition, mutations or natural variants of a polynucleotide sequence can be recombined with either the wild type or other mutations or natural variants to produce new variants. Such new variants can also be screened for improved functionality of the encoded polypeptide.

The application of the above-mentioned and similar molecular evolution methods allows the identification and selection of variants of the enzymes of the present invention which have preferred characteristics without any prior knowledge of protein structure or function, and allows the production of non-predictable but beneficial mutations or variants. There are numerous examples of the application of molecular evolution in the art for the optimisation or alteration of enzyme activity, such examples include, but are not limited to one or more of the following: optimised expression and/or activity in a host cell or in vitro, increased enzymatic activity, altered substrate and/or product specificity, increased or decreased enzymatic or structural stability, altered enzymatic activity/specificity in preferred environmental conditions, e.g. temperature, pH, substrate.

As will be apparent to a person skilled in the art, using molecular evolution tools an enzyme may be altered to improve the functionality of the enzyme.

Suitably, the nucleotide sequence encoding a enzyme (e.g. lipid acyltransferase or phospholipase) used in the invention may encode a variant enzyme (e.g. lipid acyitransferase or phospholipase), i.e. the enzyme (e.g. lipid acyltransferase or phospholipase) may contain at least one amino acid substitution, deletion or addition, when compared to a parental enzyme. Variant enzymes retain at least 1%, 2%, 3%, 5%, 10%, 15%, 20%, 30%, 40%, 50%, 60%, 70%, 80%, 90%, 95%, 97%, 99% homology with the parent enzyme. Suitable parent enzymes may include any enzyme with esterase or lipase activity. Preferably, the parent enzyme aligns to the pfam00657 consensus sequence.

In a preferable embodiment a variant lipid acyitransferase enzyme retains or incorporates at least one or more of the pfam00657 consensus sequence amino acid residues found in the GDSx, GANDY and HPT blocks.

Enzymes, such as lipases with no lipid acyltransferase activity in an aqueous environment may be mutated using molecular evolution tools to introduce or enhance the transferase activity, thereby producing a lipid acyltransferase enzyme with significant transferase activity suitable for use in the compositions and methods of the present invention.

Suitably, the nucleotide sequence encoding a lipid acyltransferase for use in any one of the methods and/or uses of the present invention may encode a lipid acyltransferase that may be a variant with enhanced enzyme activity on polar lipids, preferably when compared to the parent enzyme. Variant lipid acytransferases may have decreased activity on triglycerides, and/or monoglycerides and/or diglycerides compared with the parent enzyme.

Suitably the variant enzyme may have no activity on triglycerides and/or monoglycerides and/or diglycerides.

Alternatively, the variant enzyme may have increased activity on one or more of the following, polar lipids, phospholipids, lecithin, phosphatidylcholine.

Variants of lipid acyltransferases are known, and one or more of such variants may be suitable for use in the methods and uses according to the present invention and/or in the enzyme compositions according to the present invention. By way of example only, variants of lipid acyltransferases are described in the following references may be used in accordance with the present invention: Hilton & Buckley J. Biol. Chem. 1991 Jan. 15: 266 (2): 997-1000; Robertson et al J. Biol. Chem. 1994 Jan. 21; 269(3):2146-50; Brumlik et al J. Bacteriol. 1996 April; 178 (7): 2060-4; Peelman et al Protein Sci. 1998 March; 7(3):587-99, WO2011/045629.

### Amino Acid Sequences

The present invention also encompasses the use of amino acid sequences encoded by a nucleotide sequence which encodes a enzyme (e.g. lipid acyltransferase or phospholipase) for use in any one of the methods and/or uses of the present invention.

As used herein, the term “amino acid sequence” is synonymous with the term “polypeptide” and/or the term “protein”. In some instances, the term “amino acid sequence” is synonymous with the term “peptide”.

The amino acid sequence may be prepared/isolated from a suitable source, or it may be made synthetically or it may be prepared by use of recombinant DNA techniques.

Suitably, the amino acid sequences may be obtained from the isolated polypeptides taught herein by standard techniques.

One suitable method for determining amino acid sequences from isolated polypeptides is as follows:

Purified polypeptide may be freeze-dried and 100 μg of the freeze-dried material may be dissolved in 50 μl of a mixture of 8 M urea and 0.4 M ammonium hydrogen carbonate, pH 8.4. The dissolved protein may be denatured and reduced for 15 minutes at 50° C. following overlay with nitrogen and addition of 5 μl of 45 mM dithiothreitol. After cooling to room temperature, 5 μl of 100 mM iodoacetamide may be added for the cysteine residues to be derivatized for 15 minutes at room temperature in the dark under nitrogen.

135 μl of water and 5 μg of endoproteinase Lys-C in 5 μl of water may be added to the above reaction mixture and the digestion may be carried out at 37° C. under nitrogen for 24 hours.

The resulting peptides may be separated by reverse phase HPLC on a VYDAC C18 column (0.46×15 cm; 10 μm; The Separation Group, California, USA) using solvent A: 0.1% TFA in water and solvent B: 0.1% TFA in acetonitrile. Selected peptides may be re-chromatographed on a Develosil C18 column using the same solvent system, prior to N-terminal sequencing. Sequencing may be done using an Applied Biosystems 476A sequencer using pulsed liquid fast cycles according to the manufacturer's instructions (Applied Biosystems, California, USA).

### Sequence Identity or Sequence Homology

The present invention also encompasses the use of sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

The homologous amino acid sequence and/or nucleotide sequence should provide and/or encode a polypeptide which retains the functional activity and/or enhances the activity of the enzyme.

In the present context, a homologous sequence is taken to include an amino acid or a nucleotide sequence which may be at least 75, 85 or 90% identical, preferably at least 95 or 98% identical to the subject sequence. Typically, the homologues will comprise the same active sites etc. as the subject amino acid sequence for instance. Although homology can also be considered in terms of similarity (i.e. amino acid residues having similar chemical properties/functions), in the context of the present invention homology is expressed in terms of sequence identity.

In one embodiment, a homologous sequence is taken to include an amino acid sequence or nucleotide sequence which has one or several additions, deletions and/or substitutions compared with the subject sequence.

In one embodiment the present invention relates to a protein whose amino acid sequence is represented herein or a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In one embodiment the present invention relates to a nucleic acid sequence (or gene) encoding a protein whose amino acid sequence is represented herein or encoding a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In the present context, a homologous sequence is taken to include a nucleotide sequence which may be at least 75, 85 or 90% identical, preferably at least 95 or 98% identical to a nucleotide sequence encoding a polypeptide of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.gov), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244).

Once the software has produced an optimal alignment, it is possible to calculate % homology, preferably % sequence identity. The software typically does this as part of the sequence comparison and generates a numerical result.

Should Gap Penalties be used when determining sequence identity, then preferably the following parameters are used for pairwise alignment:

| FOR BLAST | |
|---|---|
| GAP OPEN | 0 |
| GAP EXTENSION | 0 |

| FOR CLUSTAL | DNA | PROTEIN | |
|---|---|---|---|
| WORD SIZE | 2 | 1 | K triple |
| GAP PENALTY | 15 | 10 | |
| GAP EXTENSION | 6.66 | 0.1 | |

In one embodiment, CLUSTAL may be used with the gap penalty and gap extension set as defined above.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides.

Suitably, the degree of identity with regard to a nucleotide sequence may be determined over the whole sequence.

In one embodiment the degree of amino acid sequence identity in accordance with the present invention may be suitably determined by means of computer programs known in the art, such as Vector NTI 10 (Invitrogen Corp.). For pairwise alignment the matrix used is preferably BLOSUM62 with Gap opening penalty of 10.0 and Gap extension penalty of 0.1.

Suitably, the degree of identity with regard to an amino acid sequence is determined over at least 20 contiguous amino acids, preferably over at least 30 contiguous amino acids, preferably over at least 40 contiguous amino acids, preferably over at least 50 contiguous amino acids, preferably over at least 60 contiguous amino acids.

Suitably, the degree of identity with regard to an amino acid sequence may be determined over the whole sequence.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to Table 2 below. Amino acids in the same block in the second column and preferably in the same line in the third column may be substituted for each other

TABLE 2

| | | |
|---|---|---|
| ALIPHATIC | Non-polar | G A P |
| | | I L V |
| | Polar—uncharged | C S T M |
| | | N Q |
| | Polar—charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as omithine (hereinafter referred to as Z), diaminobutyric acid omithine (hereinafter referred to as B), norleucine omithine (hereinafter referred to as O), pyriylalanine, thienylalanine, naphthylalanine and phenylglycine.

Replacements may also be made by unnatural amino acids.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

Nucleotide sequences for use in the present invention or encoding a polypeptide having the specific properties defined herein may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences.

The present invention also encompasses the use of nucleotide sequences that are complementary to the sequences discussed herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction polypeptide recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

### Hybridisation

The present invention also encompasses the use of sequences that are complementary to the sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the subject sequences discussed herein, or any derivative, fragment or derivative thereof.

The present invention also encompasses sequences that are complementary to sequences that are capable of hybridising to the nucleotide sequences discussed herein.

Hybridisation conditions are based on the melting temperature (Tm) of the nucleotide binding complex, as taught in Berger and Kimmel (1987, Guide to Molecular Cloning Techniques, Methods in Enzymology, Vol. 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical nucleotide sequences while an intermediate (or low) stringency hybridisation can be used to identify or detect similar or related polynucleotide sequences.

Preferably, the present invention encompasses the use of sequences that are complementary to sequences that are capable of hybridising under high stringency conditions or intermediate stringency conditions to nucleotide sequences encoding polypeptides having the specific properties as defined herein.

More preferably, the present invention encompasses the use of sequences that are complementary to sequences that are capable of hybridising under high stringency conditions (e.g. 65° C. and 0.1×SSC {1×SSC=0.15 M NaCl, 0.015 M Na-citrate pH 7.0}) to nucleotide sequences encoding polypeptides having the specific properties as defined herein.

The present invention also relates to the use of nucleotide sequences that can hybridise to the nucleotide sequences discussed herein (including complementary sequences of those discussed herein).

The present invention also relates to the use of nucleotide sequences that are complementary to sequences that can hybridise to the nucleotide sequences discussed herein (including complementary sequences of those discussed herein).

Also included within the scope of the present invention are the use of polynucleotide sequences that are capable of hybridising to the nucleotide sequences discussed herein under conditions of intermediate to maximal stringency. In a preferred aspect, the present invention covers the use of nucleotide sequences that can hybridise to the nucleotide sequences discussed herein, or the complement thereof, under stringent conditions (e.g. 50° C. and 0.2×SSC).

In a more preferred aspect, the present invention covers the use of nucleotide sequences that can hybridise to the nucleotide sequences discussed herein, or the complement thereof, under high stringency conditions (e.g. 65° C. and 0.1×SSC).

### Expression of Polypeptides

A nucleotide sequence for use in the present invention or for encoding a polypeptide having the specific properties as defined herein can be incorporated into a recombinant replicable vector. The vector may be used to replicate and express the nucleotide sequence, in polypeptide form, in and/or from a compatible host cell. Expression may be controlled using control sequences which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters described above.

The polypeptide produced by a host recombinant cell by expression of the nucleotide sequence may be secreted or may be contained intracellularly depending on the sequence and/or the vector used. The coding sequences can be designed with signal sequences which direct secretion of the substance coding sequences through a particular prokaryotic or eukaryotic cell membrane.

### Constructs

The term “construct”—which is synonymous with terms such as “conjugate”, “cassette” and “hybrid”—includes a nucleotide sequence encoding a polypeptide having the specific properties as defined herein for use according to the present invention directly or indirectly attached to a promoter. An example of an indirect attachment is the provision of a suitable spacer group such as an intron sequence, such as the Shi-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment.

The construct may even contain or express a marker which allows for the selection of the genetic construct.

For some applications, preferably the construct comprises at least a nucleotide sequence of the present invention or a nucleotide sequence encoding a polypeptide having the specific properties as defined herein operably linked to a promoter.

### Fusion Proteins

The enzyme for use in the present invention may be produced as a fusion protein, for example to aid in extraction and purification thereof. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the activity of the protein sequence.

Gene fusion expression systems in E. coli have been reviewed in Curr. Opin. Biotechnol. (1995) 6(5):501-6.

The amino acid sequence of a polypeptide having the specific properties as defined herein may be ligated to a non-native sequence to encode a fusion protein. For example, for screening of peptide libraries for agents capable of affecting the substance activity, it may be useful to encode a chimeric substance expressing a non-native epitope that is recognised by a commercially available antibody.

### Advantages

The method of the present invention has many advantages over prior art process, including:
Enhanced oil yield from the processing of palm fruit;
Enhanced oil yield from the sludge post clarification and/or decanting;
Reduced sludge formation—which improves the throughput of the plant and enhances the oil extraction rate;
Improved separation of the crude palm oil from the sludge;
The ability to run the clarifier or decanter at a lower temperature thus giving reduced energy consumption for the process;
Provision of a more environmentally friendly process (e.g. due to increased yields fewer hectares of land are required for plantations);
Reduced loss of oil to the fibre extract during processing; and/or
Reduced viscosity of the pressed palm fruit extract, thus reducing the requirement for water to be added. Thus the present invention can lead to a process which consumes less water.

Without wishing to be bound by theory it is believed that the high oil yields can be achieved by the method of the present invention because it destabilises or breaks the emulsion which forms during processing of palm fruit extract (e.g. pressed palm fruit extract). An emulsion forms between the oil and the water in the extract which emulsion can be relatively stable. By destabilising or breaking down this emulsion more oil can be recovered. The use of the enzymes in accordance with the present invention has surprisingly been found to be a very effective and efficient (as well as environmental friendly) way of significantly improving crude palm oil yields from palm fruit extracts (such as pressed palm fruit extracts).

A further advantage is that oil obtainable (e.g. obtained) from the methods and uses according to the present invention separates more easily and faster from the pressed palm liquid thus reducing the amount of oil in the waste stream. This advantageously allows production capacity to be increased. Another advantage is that the sludge contains less oil so that the remaining oil can be more easily separated in the separator.

The invention will now be described, by way of example only, with reference to the following Figures and Examples.

### EXAMPLES

### Example 1

### Materials and Methods

Pressed Crude Palm Oil from Paloh Palm Oil Mill, Malaysia
Obtained from pressing of digested palm mesocarp.
Water content: 64.6%
Palm fruits obtained from DuPont, Singapore

### Enzymes:

### LysoMax Oil, lot. 1781679258, DuPont Industrial Biosciences

Lecitase Ultra, produced by Novozymes, DK
Lipopan F, produced by Novozymes, DK
Nagase Phospholipase PLA2, produced by Nagase, Japan
Lipomod 699L, Pancreatic Phospholipase PLA2, produced by Biocatalysts, UK
Phospholipase GL541, Phospholipase PLA2 from Streptomyces coelicolor expressed in Bacillus subtilis.
PLDa, Phospholipase D from Streptomyces racemochromogenes, expressed in Bacillus subtilis
PLDb, Phospholipase D from Streptomyces avermitilis, expressed in Bacillus subtilis

### Experimental Procedure

In the following experiments, the effect of enzyme on separation of crude palm oil was simulated. 10 gram crude palm oil was scaled in a Wheaton glass. A magnetic bar was added and the sample was heated to 50° C. in a heating block, When the temperature had reached 50° C. enzyme was added. For experimental comparison small volume of water was added to get the same total volume of enzyme+water added. The sample was incubated for 2 hrs at 50° C. with magnetic agitation, 450 rpm. After 2 hrs incubation the sample was transferred to a 15 ml centrifuge tube. In order to inactivate the enzyme the sample was heated to 95° C. in a water bath for 10 minutes. After enzyme inactivation the samples were evaluated visually and the volume of separated oil phase was measured. The effect of enzyme treatment was calculated as % separated oil.

### Experiment 1

In this experiment a number of commercial enzymes, Lipopan F, Lecitase Ultra, Nagase PLA2, and a experimental PLA 2 (GL541) from Streptomyces violaceoruber was tested in pressed crude palm oil, and compared with the effect of an acyltransferase, LysoMax Oil (table 1)

TABLE 1
Test of enzymes with activity on phospholipids in crude palm oil

| | | 1 | 2 | 3 | 4 | 5 | 6 | 7 | 8 |
|---|---|---|---|---|---|---|---|---|---|
| Crude Palm Oil | g | 10 | 10 | 10 | 10 | 10 | 10 | 10 | 10 |
| LysoMax Oil, diluted 1:10 | μl | | 200 | 20 | | | | | |
| Lecitase Ultra 1:10 | μl | | | | 200 | | | | |
| Lipopan F 1:10 | μl | | | | | 200 | | | |
| Nagase Phospholipase PLA2 | μl | | | | | | 200 | 20 | |
| Phospholipase GL541 | μl | | | | | | | | 200 |
| Water | μl | 200 | 0 | 180 | 0 | 0 | 0 | 180 | 0 |

The experimental procedures were conducted as described under Experimental Procedure.

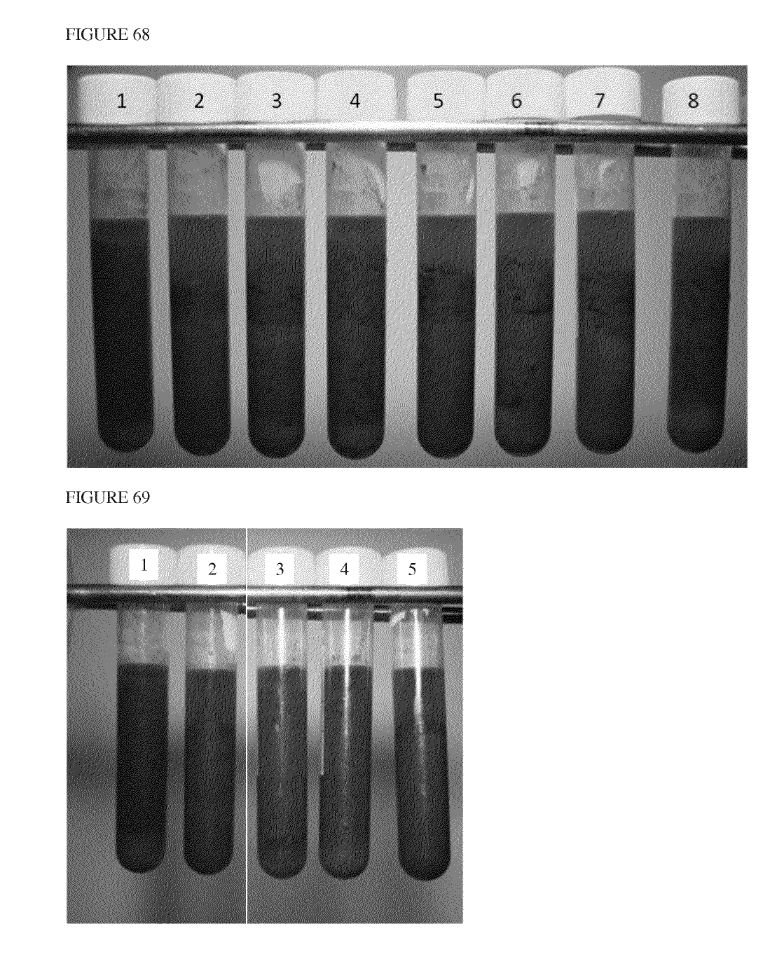

The effect of Acyltransferase and different phospholipases on oil separation is illustrated in FIG. 68, and the amount of oil separated is calculated in Table 2.

TABLE 2
Oil separation of crude palm oil samples (see Table 5)

| | LysoMax | Lecitase | Lipopan | Nagase | GL541, | Oil |
|---|---|---|---|---|---|---|
| | Oil | Ultra | F | PLA2 | PLA2 | Layer |
| sample | % | % | % | % | % | % |
| 1 | | | | | | 9.1 |
| 2 | 0.2 | | | | | 28.0 |
| 3 | 0.02 | | | | | 21.0 |
| 4 | | 0.2 | | | | 10.0 |
| 5 | | | 0.2 | | | 23.0 |
| 6 | | | | 2 | | 22.0 |
| 7 | | | | 0.2 | | 24.0 |
| 8 | | | | | 2 | 18.0 |

The results in FIG. 68 and table 2 confirm that different phospholipases, PLA1 and PLA2, have an impact on separation of pressed crude palm oil with up to 24% free oil phase, but these enzymes were not as effective as LysoMax Oil which gave 28% free oil phase. Lecitase Ultra also had an impact on the separation of pressed crude palm oil.

### Experiment 2

In this experiment other types of phospholipases including Phospholipase D were tested in pressed crude palm oil and compared with Acyltransferase Lysomax Oil according to recipes in Table 3.

TABLE 3
Test of phospholipases in crude palm oil

| | | 1 | 2 | 3 | 4 | 5 |
|---|---|---|---|---|---|---|
| Crude Palm Oil | g | 10 | 10 | 10 | 10 | |
| LysoMax Oil, diluted 1:10 | μl | | 200 | | | |
| PLDa | μl | | | 200 | | |
| PLDb | μl | | | | 200 | |
| Lipomod 699L | μl | | | | | 200 |
| Water | μl | 200 | 0 | 0 | 0 | 0 |

The experimental procedures were conducted as described under Experimental Procedure.

The effect of the enzyme tested is shown in FIG. 69 and Table 4.

TABLE 4
Oil separation of crude palm oil samples

| | LysoMax Oil | PLDa | PLDb | Lipomod 600L | Oil separation |
|---|---|---|---|---|---|
| Sample | % | % | % | % | % |
| 1 | | | | | 7.9 |
| 2 | 0.2 | | | | 27.5 |
| 3 | | 2 | | | 8.9 |
| 4 | | | 2 | | 11.0 |
| 5 | | | | 2 | 26.5 |

The results of the experiment (FIG. 69, Table 4) confirmed that Acyttransferase, LysoMax Oil and Phospholipase PLA2, Lipomod 699L have a strong impact on oil separation. Phospholipase D also has an effect on oil separation.

### Experiment 3

In this experiment Lecitase Ultra was tested in different dosages (table 5), and the oil samples after enzyme inactivation were followed by storage at 60° C. for 2 hour and oil separation measured.

TABLE 5

| | | 1 | 2 | 3 |
|---|---|---|---|---|
| Crude Palm Oil | g | 10 | 10 | 10 |
| Lecitase Ultra, #3108 | μl | 0 | 10 | 5 |
| Lecitase Ultra | % | 0 | 0.1 | 0.05 |

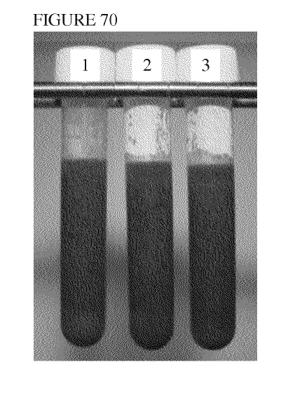

Amount of Oil separated after heat inactivation and after storage at 60° C. was measured with results in table 6 and FIG. 70.

TABLE 6

| Oil separation | | 1 | 2 | 3 |
|---|---|---|---|---|
| After heat inactivation | % | 7.6 | 14.0 | 13.2 |
| After 60 min at 60° C. | % | 11.0 | 15.7 | 14.0 |
| After 120 min at 60° C. | % | 10.8 | 16.8 | 13.3 |

The results indicate that a dosage of 0.1% Lecitase Ultra is the best under test conditions. It is also observed that at this enzyme dosage the oil separation slightly increases during storage at 60° C.

### Conclusion

Different commercial phospholipases PLA1 and PLA2, and experimental samples of PLD were tested in pressed crude palm oil with regard to impact on oil separation and compared to the effect of Acyitransferase Lysomax Oil.

The experiment confirmed that Lysomax Oil has a strong impact on the ability of oil to separate from the pressed fluid by gravity. Commercial phospholipase Lipopan F, Lipomod 699 and Nagase PLA2 also had strong impact on oil separation. Lecitase Ultra, another commercially available phospholipase also has an impact on oil separation. In addition, PLD had an impact on the ability of oil to separate from pressed crude palm oil.

### Example 2

### Materials and Methods

Pressed crude Palm Oil from Malaysia
Obtained from pressing of digested palm mesocarp

### Water Content 64.6%

Palm fruits obtained from DuPont Singapore
Water content 35%.

### Enzymes:

### LysoMax Oil, lot. 1781679258. DuPont Industrial Biosciences

Protex 15L, batch 1681480658, DuPont Industrial Biosciences
G-ZYME G999, batch 4862079064. DuPont Industrial Biosciences
Protex 50FP, batch A011161002, DuPont Industrial Biosciences

### Experimental Procedure

In the following experiments, the effect of enzyme on separation of crude palm oil was simulated. 10 gram crude palm oil was scaled in a Wheaton glass. A magnetic bar was added and the sample was heated to 50° C. in a heating block. When the temperature had reached 50° C. enzyme was added. For experimental comparison a small volume of water was added to get the same total volume. The sample was incubated for 2 hrs at 50° C. with magnetic agitation, 450 rpm. After 2 hrs incubation the sample was transferred to a 15 ml centrifuge tube. In order to inactivate the enzyme the sample was heated to 95° C. in a water bath for 10 minutes. After enzyme inactivation the samples were evaluated visually and the volume of separated oil phase was measured. The effect of enzyme treatment was calculated as % separated oil.

### Experiment 1

In the first experiment, a lipid acyl transferase (LysoMax Oil), a protease (Protex 15L), and a lysophospolipase (G-ZYME G999) were tested in a factor design as shown in Table 7.

TABLE 7

| | | 1 | 2 | 3 | 4 | 5 | 6 | 7 | 8 |
|---|---|---|---|---|---|---|---|---|---|
| Crude Palm Oil | g | 10 | 10 | 10 | 10 | 10 | 10 | 10 | 10 |
| LysoMax Oil | μl | | 100 | | 100 | | 100 | | 100 |
| Protex 15L | μl | | | 100 | 100 | | | 100 | 100 |
| G-ZYME G999 | μl | | | | | 100 | 100 | 100 | 100 |
| Water | μl | 300 | 200 | 200 | 100 | 200 | 100 | 100 | 0 |

The samples were prepared according to the procedure mentioned in Experimental.

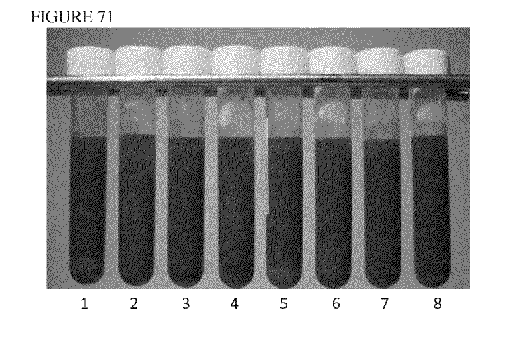

After heat inactivation of the enzymes, the samples were compared visually (FIG. 71) and the volume % of separated oil was measured (Table 8)

TABLE 8
Evaluation of oil separation after enzyme inactivation

| Sample | LysoMax Oil, | Protex 15L, | G-ZYME G999, | Oil layer, |
|---|---|---|---|---|
| no | % | % | % | % |
| 1 | 0 | 0 | 0 | 5.0 |
| 2 | 1 | 0 | 0 | 25.0 |
| 3 | 0 | 1 | 0 | 8.2 |
| 4 | 1 | 1 | 0 | 18.0 |
| 5 | 0 | 0 | 1 | 6.6 |
| 6 | 1 | 0 | 1 | 11.5 |
| 7 | 0 | 1 | 1 | 6.7 |
| 8 | 1 | 1 | 1 | 7.4 |

The results shown in Table 8 and FIG. 71 clearly indicate that the enzyme treatment had an impact on oil separation. The sample treated with LysoMax Oil showed much better results than the samples treated with other enzymes.

### Experiment 2

The effect of LysoMax Oil on oil separation of crude palm oil was investigated with lower enzyme dosage from 1% down to 0.01%. In this experiment another protease, Protex 50FP, was also tested in recipes shown in Table 9.

TABLE 9

| | | 1 | 2 | 3 | 4 | 5 | 6 | 7 |
|---|---|---|---|---|---|---|---|---|
| Crude Palm Oil | g | 10 | 10 | 10 | 10 | 10 | 10 | 10 |
| LysoMax Oil | μl | | 100 | | | | | |
| LysoMax Oil diluted | μl | | | 200 | 100 | 50 | 10 | |
| 1:10 | | | | | | | | |
| Protex 50 FP diluted | μl | | | | | | | 100 |
| 1:10 | | | | | | | | |
| Water | μl | 200 | 100 | 0 | 100 | 150 | 190 | 100 |
| LysoMax Oil | % | 0 | 1 | 0.2 | 0.1 | 0.05 | 0.01 | |

The experimental procedures were conducted as described under Experimental.

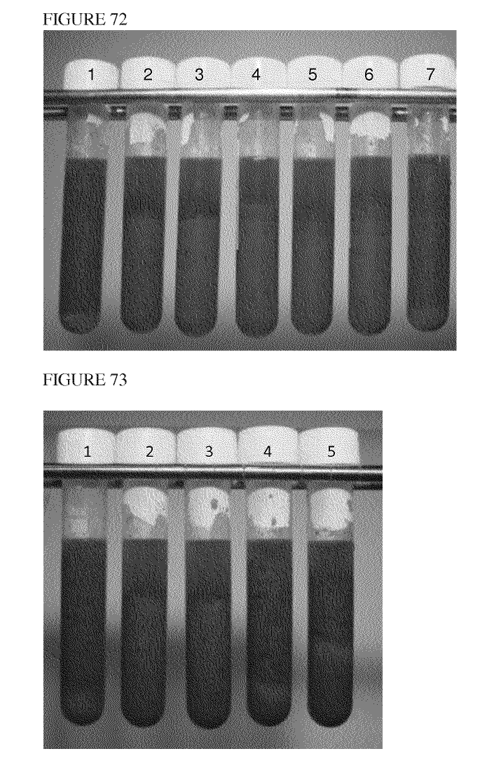

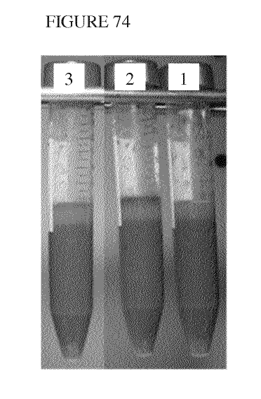

The effect of enzyme treatment on oil separation is illustrated in FIG. 72 and the amount of separated oil is shown in Table 10.

TABLE 10
Oil separation of crude palm oil after enzyme inactivation

| | LysoMax Oil, | Protex 50FP | |
|---|---|---|---|
| Sample | % | % | % Oil |
| 1 | 0 | | 8.3 |
| 2 | 1 | | 29.0 |
| 3 | 0.2 | | 27.4 |
| 4 | 0.1 | | 28.4 |
| 5 | 0.05 | | 28.6 |
| 6 | 0.01 | | 20.6 |
| 7 | | 0.1 | 9.6 |

The results in FIG. 72 and Table 10 confirmed that LysoMax Oil had a significant impact on oil separation. Lower dosage of enzyme gave the same oil separation down to 0.05% LysoMax Oil.

### Experiment 3

In this experiment the effect of LysoMax Oil on oil separation as a function of storage at 60° C. was investigated (Table 11). In this experiment no water was added to adjust for different volume of enzyme addition.

TABLE 11

| | | 1 | 2 | 3 | 4 | 5 |
|---|---|---|---|---|---|---|
| Crude Palm Oil | g | 10 | 10 | 10 | 10 | 10 |
| LysoMax Oil | μl | 0 | 100 | 50 | 10 | 5 |
| LysoMax Oil | % | 0 | 1 | 0.5 | 0.1 | 0.05 |

The experimental procedures were conducted as described under Experimental Procedures above with the modification that after enzyme inactivation the samples were placed in a heating cabinet at 60° C., and the amount of oil separated after 1 and 2 hr was measured.

The effect of Lysomax oil on oil separation is illustrated in FIG. 73.

The amount of oil separated after heat inactivation after storage at 60° C. was measured and with the results in Table 12.

TABLE 12

| Oil separation | | 1 | 2 | 3 | 4 | 5 |
|---|---|---|---|---|---|---|
| After heat inactivation | % | 7.6 | 29.3 | 29.7 | 14.1 | 16.5 |
| After 60 min at 60° C. | % | 11.0 | 30.7 | 30.1 | 16.5 | 20.7 |
| After 120 min at 60° C. | % | 10.8 | 30.2 | 30.2 | 17.6 | 23.0 |

The experiment confirmed that Lysomax Oil has a strong impact on the ability of oil to separate from the pressed fluid by gravity. Most of the oil (29.3%) is already separated after 10 minutes heat inactivation and during storage at 60° C. not much more oil is separated. The control without enzyme added only gave 7.6% oil separation, but during storage this raised to approx 11%.

Addition of 1% or 0.5% LysoMax Oil gave the same oil separation. The results confirmed that pressed oil treated with Lysomax Oil gave a significant and immediately improved oil separation.

### Conclusion

Acyltransferase, lyso-phospholipase and protease, were tested in crude palm oil with regard to impact on oil separation.

The experiments showed that Acyltransferase, LysoMax Oil, had a surprisingly strong impact on oil separation. Without wishing to be bound by theory the highly advantageous effect of acyitransferase LysoMax Oil might be explained by the fact that this enzyme instead of producing free fatty acids catalyzes a transfer reaction to other components in crude palm oil such as phytosterols.

### Example 3

### Materials

Palm sludge, ‘Underflow’ obtained from Malaysia

### Enzymes:

LysoMax Oil, batch 1781679258. DuPont Industrial Biosciences
G-ZYME G999, batch 4862079064, DuPont Industrial Biosciences

### Experimental Procedures

10 gram sludge underflow was scaled in a 20 ml Wheaton glass and heated to 50° C. with magnetic agitation.

Enzymes were added and the sample was incubated at 50° C. for 2 hr with agitation.

The sample was transferred to a 15 centrifuge tube and centrifuged at 48 rcf and 60° C. for 5 minutes. The sample was evaluated visually.

The sample was then centrifuged at 3050 rcf for 10 minutes.

The sample was evaluated visually and oil separation measured.

After centrifugation the upper liquid layer was drained and the sediment isolated.

Weight of the sediment was determined gravimetrically.

### Experiment 1

The effect of Acyltransferase, LysoMax Oil and lyso-phospholipase G-ZYME G99 was tested in a factorial design as shown in table 13.

The test was conducted as described under Experimental Procedures (above).

TABLE 13

| | | 1 | 2 | 3 |
|---|---|---|---|---|
| Underflow, sludge | g | 10 | 10 | 10 |
| LysoMax Oil | μl | | 100 | |
| G-ZYME G999 | μl | | | 100 |
| Water | μl | 300 | 200 | 200 |

After enzyme reaction the samples were transferred to 15 ml centrifuge tubes. The viscosity of the samples were however quite different, so for the samples with high viscosity less product was transferred to the tubes because it was difficult to empty the Wheaton glass.

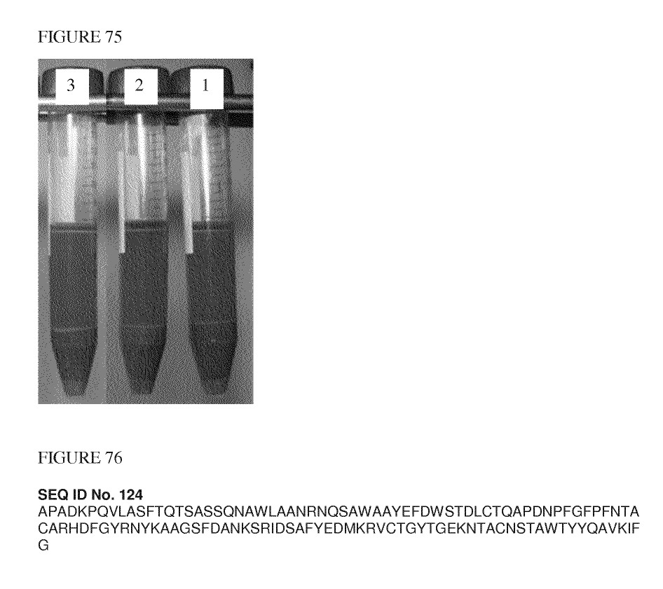

The samples after a first centrifugation at 48 rcf for 5 minutes are shown in FIG. 75.

Lysomax Oil and G-ZYME G999 also had positive effect on oil separation.

After a second centrifugation at 3050 rcf for 10 min at 60° C. (FIG. 76) the volume of the oil was measured relative to the total volume. The upper liquid phase was drained from the sediment. And the weight of the sediment was determined relative to the total amount of sample.

The amount of oil separated from the sludge is impacted by the enzyme treatment, and G-zyme G999 and LysoMax contributed to increased oil separation see FIG. 76 and (table 14).

TABLE 14
Oil separation after enzyme treatment of Palm sludge,

| Test | LysoMax Oil | G-ZYME G999 | Oil separation |
|---|---|---|---|
| no | % | % | % |
| 1 | 0 | 0 | 3.9 |
| 2 | 1 | 0 | 5.0 |
| 3 | 0 | 1 | 4.4 |

### Experiment 2

The effect of enzyme on viscosity of palm sludge underflow was tested according to recipes in table 15.

TABLE 15

| | | 1 | 2 |
|---|---|---|---|
| Underflow, sludge. | g | 10 | 10 |
| LysoMax Oil | μl | | 100 |
| Water | μl | 100 | 0 |
| Viscosity, 24° C. | mPa · s | 2.31 | 1.98 |

The experiment was conducted according to the procedure described under Experimental Procedures (above).

After 2 hours of incubation the enzymes in the samples were inactivated by placing the samples in a water bath at 95° C. for 10 minutes. After cooling to ambient temperature the samples were centrifuged at 15 rcf for 5 minutes. Viscosity of the water phase was measured at 24° C.

The lipid in the enzyme treated samples were analysed by thin layer chromatography (TLC) and the amount of phospholipids in the sludge was determined (table 16)

TABLE 16
TLC determination of Phosphatidylcholine(PC) phosphatidylinositol(PI),
phosphatidic acid(PA) and Phosphatidylethanolamine(PE) in sludge
treated with enzymes.

| | | PC | PI | PA | PE |
|---|---|---|---|---|---|
| Test | | ppm | ppm | ppm | ppm |
| 1 | Control | 65.3 | 12.0 | 20.8 | 18.5 |
| 2 | LysoMax Oil | 0.0 | 0.0 | 0.0 | 0.0 |

The TLC analysis confirms that that the palm sludge contains phospholipids and that LysoMax Oil is able to completely degrade the phospholipids.

### Conclusion

One of the challenges in palm oil production is the isolation of the oil from water and other material in the clarifier. The crude oil coming from the oil press contains oil, water and other plant material which can create a rather stable emulsion. Such an emulsion can delay or even prevent oil separation, and will cause loss of oil.

In the current study the effect of different enzymes on oil separation from the water phase (sludge) was investigated. Acyltransferase (LysoMax Oil) and lyso-phospholipase (G-Zyme G999) improved oil separation.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for improving crude palm oil yields or separating the crude palm oil from the sludge or a combination thereof in palm fruit processing comprising:
a. admixing a palm fruit or a part of the palm fruit that remains Post stripping the fruit from the bunches or a palm fruit extract and an enzyme, which enzyme degrades a phospholipid present in said palm fruit or the part of the palm fruit that remains post stripping the fruit from the bunches or palm fruit extract, and
b. incubating the admixture at about 45° C. to about 95° C. for about 15 minutes to about 6 hours.

2. A method according to claim 1, wherein the admixture is incubated for about 2 hours.

3. A method according to claim 1 wherein method further comprises separating crude palm oil from sludge.

4. A method according to claim 1 wherein the crude palm oil is separated from the sludge by clarifying, decanting or a combination thereof.

5. A method according to any one of claims 1 to 4 wherein the crude palm oil is separated from the sludge by centrifugation.

6. A method according to any one of claims 1 to 4 wherein the palm fruit extract is a pressed palm fruit extract.

7. A method according to any one of claims 1 to 4 wherein the admixing occurs during digestion of one or more palm fruit(s) or the part(s) of one or more palm fruit(s) that are remaining post stripping the fruit(s) from the bunches prior to pressing.

8. A method according to any one of claims 1 to 4, wherein the palm fruit extract is a pressed palm fruit extract and the admixing occurs prior to or during the separation step.

9. A method according to claim 1, wherein prior to admixing the palm fruit extract and the enzyme, the method comprises separating crude palm oil from sludge, wherein the sludge so produced is a fraction from palm fruit extract that is admixed with the enzyme.

10. A method according to claim 9 wherein the enzyme is admixed with the sludge prior to further recovery of residual oil from the sludge.

11. A method according to any one of claims 1 to 4 wherein the enzyme may be selected from the group consisting of: a lipid acyltransferase, a phospholipase A2, a phospholipase A1, a phospholipase B, and a phospholipase D.

12. A method according to any one of claims 1 to 4 wherein the enzyme may be selected from the group consisting of: a lipid acyltransferase, a phospholipase A2, and a phospholipase A1.

13. A method according to any one of claims 1 to 4 wherein the enzyme is a lipid acyltransferase.

14. A method according to any one of claims 1 to 4 wherein the enzyme that degrades a phospholipid may be used in combination with an enzyme composition comprising one or more of the following enzyme activities: cellulase activity, mannanase activity, pectinase activity, xylanase activity, glucuronidase activity, or galactanase activity.

15-22. (canceled)
